(19) United States
(12) Patent Application Publication
(10) Pub. No.: US 2024/0252656 A1
(43) Pub. Date: Aug. 1, 2024

(54) SALT FORMS OF IRAK4 DEGRADERS

(71) Applicant: Kymera Therapeutics, Inc., Watertown, MA (US)

(72) Inventor: Christopher P. HENCKEN, Watertown, MA (US)

(21) Appl. No.: 18/486,889

(22) Filed: Oct. 13, 2023

Related U.S. Application Data
(60) Provisional application No. 63/379,391, filed on Oct. 13, 2022.

(51) Int. Cl. A61K 47/55

(52) U.S. Cl. CPC A61K 47/55

(57) ABSTRACT

The present disclosure relates generally to various salt forms and compositions thereof useful for the modulation of interleukin-1 receptor-associated kinase 4 (“IRAK4”) via ubiquitination and/or degradation and uses of the same in the treatment various diseases.

### CROSS-REFERENCE TO RELATED APPLICATIONS

This application claims the benefit of priority to U.S. Provisional Appl. No. 63/379,391, filed Oct. 13, 2022, the content of which is herein incorporated by reference.

### FIELD OF THE INVENTION

The present disclosure relates generally to various salt forms and compositions thereof useful for the modulation of interleukin-1 receptor-associated kinase 4 (“IRAK4”) via ubiquitination and/or degradation and uses of the same in the treatment various diseases.

### BACKGROUND

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; 26169771.3.BUSINESS 2 of 1812 397731-010US (170174) Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

An ongoing need exists in the art for effective treatments for disease, especially autoinflammatory and autoimmune diseases with high unmet medical need. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as interleukin-1 receptor-associated kinase 4 (“IRAK4”) hold promise as therapeutic agents. Accordingly, there remains a need to find compounds that are IRAK4 degraders useful as therapeutic agents.

### SUMMARY OF THE INVENTION

It has now been found that novel salt forms of 5-((1R,4R)-2-oxa-5-azabicyclo[2.2.1]heptan-5-yl)-N-(3-(difluoromethyl)-1-((1r,4R)-4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)cyclohexyl)-1H-pyrazol-4-yl)pyrazolo[1,5-a]pyrimidine-3-carboxamide, as described in the present disclosure, and compositions thereof, are useful modulators of targeted ubiquitination of IRAK4 kinase and exhibit desirable characteristics for the same. In general, salt forms and pharmaceutically acceptable compositions thereof, are useful for treating or lessening the severity of a variety of diseases or disorders as described in detail herein.

### BRIEF DESCRIPTION OF THE DRAWINGS

FIG. 1A depicts an XRPD pattern overlay of Form A of compound 2: a) dried solid; and b)-d) wet cakes.

FIG. 1B depicts an DSC-TGA overlay of Form A of compound 2.

FIG. 2A depicts an XRPD pattern overlay of Form A of compound 3: a) dried solid; and b)-c) wet cakes.

FIG. 2B depicts an DSC-TGA overlay of Form A of compound 3.

FIG. 3A depicts an XRPD pattern overlay of Form A of compound 4: a) p-toluenesulfonic acid monohydrate; b)-d) wet cakes; and e) dried solid.

FIG. 3B depicts an DSC-TGA overlay of Form A of compound 4.

FIG. 4A depicts an XRPD pattern overlay of Form A of compound 5: a) dried solid; and b)-c) wet cakes.

FIG. 4B depicts an DSC-TGA overlay of Form A of compound 5.

FIG. 5A depicts an XRPD pattern overlay of Form A of compound 6: a)-d) wet cakes; and e) dried solid.

FIG. 5B depicts an DSC-TGA overlay of Form A of compound 6.

FIG. 6A depicts an XRPD pattern overlay of Form A of compound 7: a) maleic acid monohydrate; b)-d) wet cakes; and e) dried solid.

FIG. 6B depicts an DSC-TGA overlay of Form A of compound 7.

FIG. 7A depicts an XRPD pattern overlay of Form A of compound 8: a) L-tartaric acid; b)-e) wet cakes; and f) dried solid.

FIG. 7B depicts an DSC-TGA overlay of Form A of compound 8.

FIG. 8A depicts an XRPD pattern overlay of Form A of compound 9: a) fumaric acid; b)-g) wet cakes; and g)-h) dried solids.

FIG. 8B depicts an DSC-TGA overlay of Form A of compound 9.

FIG. 9A depicts an XRPD pattern overlay of Form A of compound 10: a) citric acid; b)-e) wet cakes; and f) dried solid.

FIG. 9B depicts an DSC-TGA overlay of Form A of compound 10.

FIG. 10A depicts an XRPD pattern overlay of Form A of compound 11: a) glycolic acid; b)-e) wet cakes; and f) dried solid.

FIG. 10B depicts an DSC-TGA overlay of Form A of compound 11.

FIG. 11A depicts an XRPD pattern overlay of Form A of compound 12: a) L-malic acid; b)-g) wet cakes; and g)-h) dried solids.

FIG. 11B depicts an DSC-TGA overlay of Form A of compound 12.

FIG. 12A depicts an XRPD pattern overlay of Form A of compound 13: a) hippuric acid; b)-e) wet cakes; and f) dried solid.

FIG. 12B depicts an DSC-TGA overlay of Form A of compound 13.

FIG. 13A depicts an XRPD pattern overlay of Form A of compound 14: a) L-lactic acid; b)-e) wet cakes; and f) dried solid.

FIG. 13B depicts an DSC-TGA overlay of Form A of compound 14.

FIG. 14A depicts an XRPD pattern overlay of Form A of compound 15: a) benzoic acid; b)-e) wet cakes; and f) dried solid.

FIG. 14B depicts an DSC-TGA overlay of Form A of compound 15.

FIG. 15A depicts an XRPD pattern overlay of Form A, B, and C of compound 16: a) succinic acid; b)-f) wet cakes; g) Form B dried solid; h) Form C dried solid; and i) Form A dried solid.

FIG. 15B depicts an DSC-TGA overlay of Form B of compound 16.

FIG. 15C depicts an DSC-TGA overlay of Form C of compound 16.

FIG. 15D depicts an DSC-TGA overlay of Form A of compound 16.

### DETAILED DESCRIPTION OF THE INVENTION

### General Description of Certain Aspects of the Invention:

The present disclosure is based at least in part on the identification of a compound that modulates targeted ubiquitination of IRAK4 kinase and methods of using the same to treat an IRAK4 mediated disease, disorder or condition in a patient in need thereof. Disclosed herein is compound 1, and salt forms thereof:

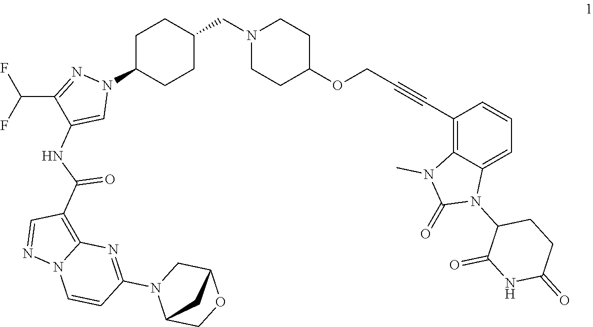 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

Compound 1, 5-((1R,4R)-2-oxa-5-azabicyclo[2.2.1]heptan-5-yl)-N-(3-(difluoromethyl)-1-((1r,4R)-4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)cyclohexyl)-1H-pyrazol-4-yl)pyrazolo[1,5-a]pyrimidine-3-carboxamide, is active as a modulator of targeted ubiquitination of IRAK4. Compound 1 is designated I-417 and its synthesis is described in U.S. Pat. No. 11,352,350, the entirety of which is herein incorporated by reference.

It would be desirable to provide a solid form of compound 1 (e.g., as a salt thereof) that imparts characteristics such as improved aqueous solubility, stability and ease of formulation. Accordingly, the present disclosure provides salt forms of compound 1.

### Salt Forms of Compound 1

In some embodiments, an acid and compound 1 are ionically bonded to form a salt form of compound 1, as described below. It is contemplated that the salt form of compound 1 can exist in a variety of physical forms. For example, a salt form of compound 1 can be in solution, suspension, or in solid form. In certain embodiments, a salt form of compound 1 is in solid form. When a salt form of compound 1 is in solid form, said compound may be amorphous, crystalline, or a mixture thereof. In certain embodiments, a salt solid form of compound 1 is crystalline. Exemplary such salt forms of compound 1 are described in more detail below.

In some embodiments, the present disclosure provides a salt form of compound 1 substantially free of impurities. Such extraneous matter may include different forms of compound 1 or any other impurities that may result from the preparation of, and/or isolation of, compound 1. In certain embodiments, at least about 85% by weight of a salt form of compound 1 is present. In certain embodiments, at least about 90% by weight of a salt form of compound 1 is present. In still other embodiments of the disclosure, at least about 95% by weight of a salt form of compound 1 is present.

According to one embodiment, a salt form of compound 1 is present in an amount of at least about 80, 85, 90, 95, 97, 97.5, 98.0, 98.5, 99, 99.5, 99.8 weight percent where the percentages are based on the total weight of the composition. According to another embodiment, a salt form of compound 1 contains no more than about 20.0 area percent HPLC of total organic impurities, no more than about 15.0 area percent HPLC of total organic impurities, no more than about 10.0 area percent HPLC of total organic impurities, no more than about 5.0 area percent HPLC of total organic impurities, no more than about 20.0 area percent HPLC of total organic impurities, no more than about 20.0 area percent HPLC of total organic impurities, or no more than about 1.5 area percent HPLC total organic impurities relative to the total area of the HPLC chromatogram. In other embodiments, a salt form of compound 1 contains no more than about 10% area percent HPLC of any single impurity, no more than about 5% area percent HPLC of any single impurity, or no more than about 1% area percent HPLC of any single impurity, relative to the total area of the HPLC chromatogram.

The structure depicted for a salt form of compound 1 is also meant to include all tautomeric salt forms of compound 1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In certain embodiments, the salt form of compound 1 is a crystalline solid. In other embodiments, the salt form of compound 1 is a crystalline solid substantially free of amorphous compound 1. In still other embodiments, the salt form of compound 1 is a crystalline solid substantially free of other crystalline forms of compound 1 (e.g., crystalline free base and salt forms). In certain embodiments, at least about 95% by weight of crystalline salt form of compound 1 is present. In still other embodiments of the disclosure, at least about 99% by weight of crystalline salt form of compound 1 is present.

In some embodiments, the present invention provides a composition comprising a salt form compound 1 and a pharmaceutically acceptable carrier or excipient.

In some embodiments, the present disclosure provides a method of modulating IRAK4 kinase in a patient comprising administering to said patient a salt form of compound 1 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient a salt form of compound 1 or a composition thereof. In some embodiments, the present disclosure provides a method of treating a disease, disorder or condition in a patient, comprising administering to said patient a salt form of compound 1 or a composition thereof, wherein the disease, disorder or condition is mediated by IRAK4. In some embodiments, the present disclosure provides a method of treating an autoimmune or inflammatory disorder in a patient, comprising administering to said patient a salt form of compound 1 or a composition thereof.

As described herein, it has been found that the salt forms of compound 1 can exist in at least fifteen distinct polymorphic forms. In certain embodiments, the present disclosure provides a polymorphic form of compound 1 referred to herein as Form A of compound 2. In certain embodiments, the present disclosure provides a polymorphic form of compound 1 referred to herein as Form A of compound 3. In certain embodiments, the present disclosure provides a polymorphic form of compound 1 referred to herein as Form A of compound 4. In certain embodiments, the present disclosure provides a polymorphic form of compound 1 referred to herein as Form A of compound 5. In certain embodiments, the present disclosure provides a polymorphic form of compound 1 referred to herein as Form A of compound 6. In certain embodiments, the present disclosure provides a polymorphic form of compound 1 referred to herein as Form A of compound 7. In certain embodiments, the present disclosure provides a polymorphic form of compound 1 referred to herein as Form A of compound 8. In certain embodiments, the present disclosure provides a polymorphic form of compound 1 referred to herein as Form A of compound 9. In certain embodiments, the present disclosure provides a polymorphic form of compound 1 referred to herein as Form A of compound 10. In certain embodiments, the present disclosure provides a polymorphic form of compound 1 referred to herein as Form A of compound 11. In certain embodiments, the present disclosure provides a polymorphic form of compound 1 referred to herein as Form A of compound 12. In certain embodiments, the present disclosure provides a polymorphic form of compound 1 referred to herein as Form A of compound 13. In certain embodiments, the present disclosure provides a polymorphic form of compound 1 referred to herein as Form A of compound 14. In certain embodiments, the present disclosure provides a polymorphic form of compound 1 referred to herein as Form A of compound 15. In certain embodiments, the present disclosure provides a polymorphic form of compound 1 referred to herein as Form A of compound 16. In certain embodiments, the present disclosure provides a polymorphic form of compound 1 referred to herein as Form B of compound 16. In certain embodiments, the present disclosure provides a polymorphic form of compound 1 referred to herein as Form C of compound 16.

### Compound 2 (Sulfuric Acid Salts of Compound 1)

According to one embodiment, the present disclosure provides a sulfuric acid salt of compound 1, represented by compound 2:

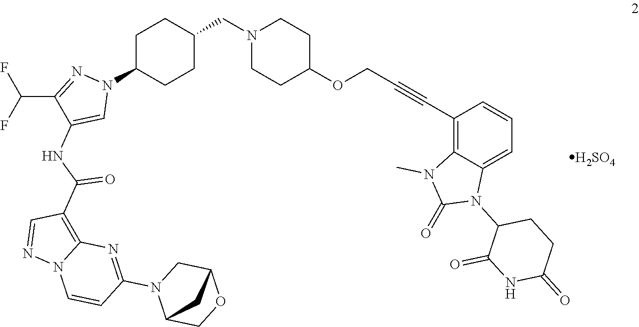

It will be appreciated by one of ordinary skill in the art that the sulfuric acid and compound 1 are ionically bonded to form compound 2. In some embodiments, compound 2 is a hydrogen sulfate salt. In some embodiments, compound 2 is a sulfate salt. In some embodiments, compound 2 exists as a mixture of hydrogen sulfate and sulfate salts. It is contemplated that compound 2 can exist in a variety of physical forms. For example, compound 2 can be in solution, suspension, or in solid form. In certain embodiments, compound 2 is in solid form. When compound 2 is in solid form, said compound may be amorphous, crystalline, or a mixture thereof. Exemplary solid forms are described in more detail below.

In some embodiments, the present disclosure provides compound 2 substantially free of impurities. Such impurities or extraneous matter may include different forms of compound 2, residual solvents, or any other impurities that may result from the preparation of, and/or isolation of, compound 2. In certain embodiments, at least about 95% by weight of a form of compound 2 is present. In still other embodiments of the disclosure, at least about 99% by weight of a form of compound 2 is present.

According to one embodiment, a form of compound 2 is present in an amount of at least about 97, 97.5, 98.0, 98.5, 99, 99.5, 99.8 weight percent where the percentages are based on the total weight of the composition. According to another embodiment, a form of compound 2 contains no more than about 3.0 area percent HPLC of total organic impurities and, in certain embodiments, no more than about 1.5 area percent HPLC total organic impurities relative to the total area of the HPLC chromatogram. In other embodiments, a form of compound 2 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 2 is also meant to include all tautomeric forms of compound 2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 2 can exist in a variety of solid forms. Exemplary such forms include polymorphs such as those described herein.

In certain embodiments, compound 2 is a crystalline solid. In other embodiments, compound 2 is a crystalline solid substantially free of amorphous compound 2. As used herein, the term “substantially free of amorphous compound 2” means that the compound contains no significant amount of amorphous compound 2. In certain embodiments, at least about 95% by weight of crystalline compound 2 is present. In still other embodiments of the disclosure, at least about 99% by weight of crystalline compound 2 is present.

It has been found that compound 2 can exist in at least one distinct polymorphic form. In certain embodiments, the present disclosure provides a polymorphic form of compound 2 referred to herein as Form A.

### Form A of Compound 2

Methods for preparing Form A of compound 2 are described infra.

In some embodiments, the disclosure provides compound 2:

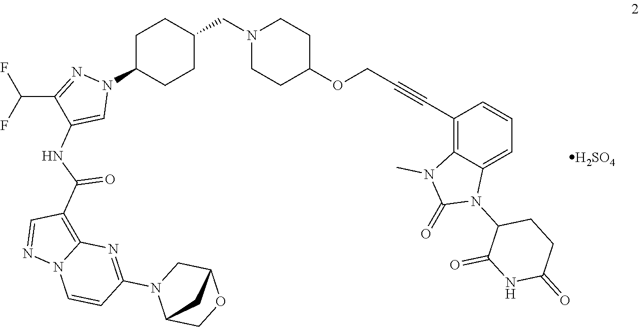

wherein said compound is crystalline. In some embodiments, the present disclosure provides compound 2, wherein said compound is substantially free of amorphous compound 2. In some embodiments, the present disclosure provides compound 2, wherein said compound is substantially free of amorphous compound 1. In some embodiments, the present disclosure provides compound 2, wherein said compound is substantially free of a crystalline form of compound 1. In some embodiments, the present disclosure provides compound 2, wherein said compound is substantially free of impurities.

In some embodiments, Form A of compound 2 is characterized by one or more peaks in its XRPD pattern selected from those at 16.5, 17.5, and 19.7±0.5 degree 2-theta. In some embodiments, Form A of compound 2 is characterized by one or more peaks in its XRPD pattern selected from those at 16.5, 17.5, and 19.7±0.4 degree 2-theta. In some embodiments, Form A of compound 2 is characterized by one or more peaks in its XRPD pattern selected from those at 16.5, 17.5, and 19.7±0.3 degree 2-theta. In some embodiments, Form A of compound 2 is characterized by one or more peaks in its XRPD pattern selected from those at 16.5, 17.5, and 19.7±0.2 degree 2-theta.

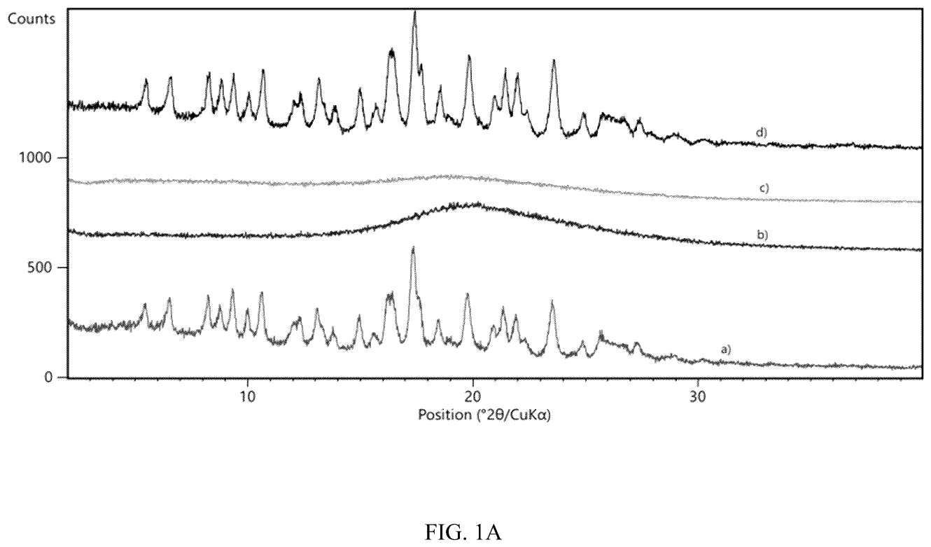

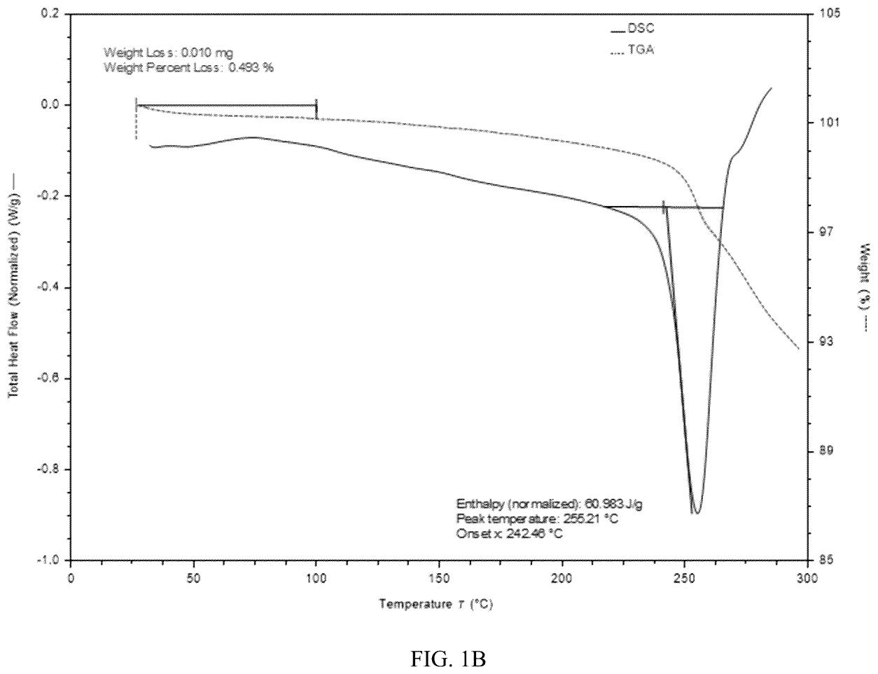

In some embodiments, the present disclosure provides compound 2, wherein said compound has an XRPD substantially similar (e.g., having peaks ±0.2 degree 2-theta) to that depicted in FIG. 1A.

In some embodiments, the present disclosure provides compound 2, wherein said compound has a DSC-TGA substantially similar (e.g., having an endothermic DSC event onset at 240-245° C.) to that depicted in FIG. 1B. In some embodiments, the present disclosure provides compound 2, wherein said compound has a DSC event and/or a TGA weight loss as described in Table 6.

In some embodiments, the present disclosure provides a composition comprising compound 2 and a pharmaceutically acceptable carrier or excipient.

In some embodiments, the present disclosure provides a method of modulating IRAK4 kinase in a patient comprising administering to said patient compound 2 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 2 or a composition thereof. In some embodiments, the present disclosure provides a method of treating a disease, disorder or condition in a patient, comprising administering to said patient compound 2 or a composition thereof, wherein the disease, disorder or condition is mediated by IRAK4. In some embodiments, the present disclosure provides a method of treating an autoimmune or inflammatory disorder in a patient, comprising administering to said patient compound 2 or a composition thereof.

### Compound 3 (Benzenesulfonic Acid Salts of Compound 1)

According to one embodiment, the present disclosure provides a benzenesulfonic acid salt of compound 1, represented by compound 3:

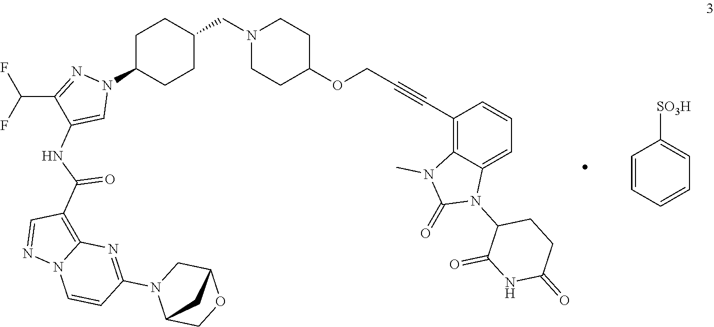 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=S(=O)(O)c1ccccc1]

It will be appreciated by one of ordinary skill in the art that the benzenesulfonic acid and compound 1 are ionically bonded to form compound 3. In some embodiments, compound 3 is a benzenesulfate salt. It is contemplated that compound 3 can exist in a variety of physical forms. For example, compound 3 can be in solution, suspension, or in solid form. In certain embodiments, compound 3 is in solid form. When compound 3 is in solid form, said compound may be amorphous, crystalline, or a mixture thereof. Exemplary solid forms are described in more detail below.

In some embodiments, the present disclosure provides compound 3 substantially free of impurities. Such impurities or extraneous matter may include different forms of compound 3, residual solvents, or any other impurities that may result from the preparation of, and/or isolation of, compound 3. In certain embodiments, at least about 95% by weight of a form of compound 3 is present. In still other embodiments of the disclosure, at least about 99% by weight of a form of compound 3 is present.

According to one embodiment, a form of compound 3 is present in an amount of at least about 97, 97.5, 98.0, 98.5, 99, 99.5, 99.8 weight percent where the percentages are based on the total weight of the composition. According to another embodiment, a form of compound 3 contains no more than about 3.0 area percent HPLC of total organic impurities and, in certain embodiments, no more than about 1.5 area percent HPLC total organic impurities relative to the total area of the HPLC chromatogram. In other embodiments, a form of compound 3 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 3 is also meant to include all tautomeric forms of compound 3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 3 can exist in a variety of solid forms. Exemplary such forms include polymorphs such as those described herein.

In certain embodiments, compound 3 is a crystalline solid. In other embodiments, compound 3 is a crystalline solid substantially free of amorphous compound 3. As used herein, the term “substantially free of amorphous compound 3” means that the compound contains no significant amount of amorphous compound 3. In certain embodiments, at least about 95% by weight of crystalline compound 3 is present. In still other embodiments of the disclosure, at least about 99% by weight of crystalline compound 3 is present.

It has been found that compound 3 can exist in at least one distinct polymorphic form. In certain embodiments, the present disclosure provides a polymorphic form of compound 3 referred to herein as Form A.

### Form A of Compound 3

Methods for preparing Form A of compound 3 are described infra.

In some embodiments, the disclosure provides compound 3:

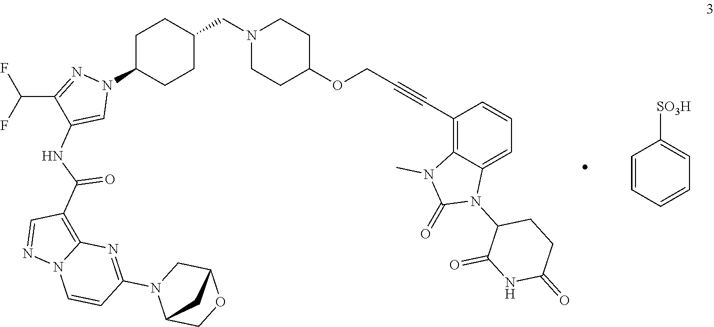 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=S(=O)(O)c1ccccc1]

wherein said compound is crystalline. In some embodiments, the present disclosure provides compound 3, wherein said compound is substantially free of amorphous compound 3. In some embodiments, the present disclosure provides compound 3, wherein said compound is substantially free of amorphous compound 1. In some embodiments, the present disclosure provides compound 3, wherein said compound is substantially free of a crystalline form of compound 1. In some embodiments, the present disclosure provides compound 3, wherein said compound is substantially free of impurities.

In some embodiments, Form A of compound 3 is characterized by one or more peaks in its XRPD pattern selected from those at 5.6, 11.5, and 16.5±0.5 degree 2-theta. In some embodiments, Form A of compound 3 is characterized by one or more peaks in its XRPD pattern selected from those at 5.6, 11.5, and 16.5±0.4 degree 2-theta. In some embodiments, Form A of compound 3 is characterized by one or more peaks in its XRPD pattern selected from those at 5.6, 11.5, and 16.5±0.3 degree 2-theta. In some embodiments, Form A of compound 3 is characterized by one or more peaks in its XRPD pattern selected from those at 5.6, 11.5, and 16.5±0.2 degree 2-theta.

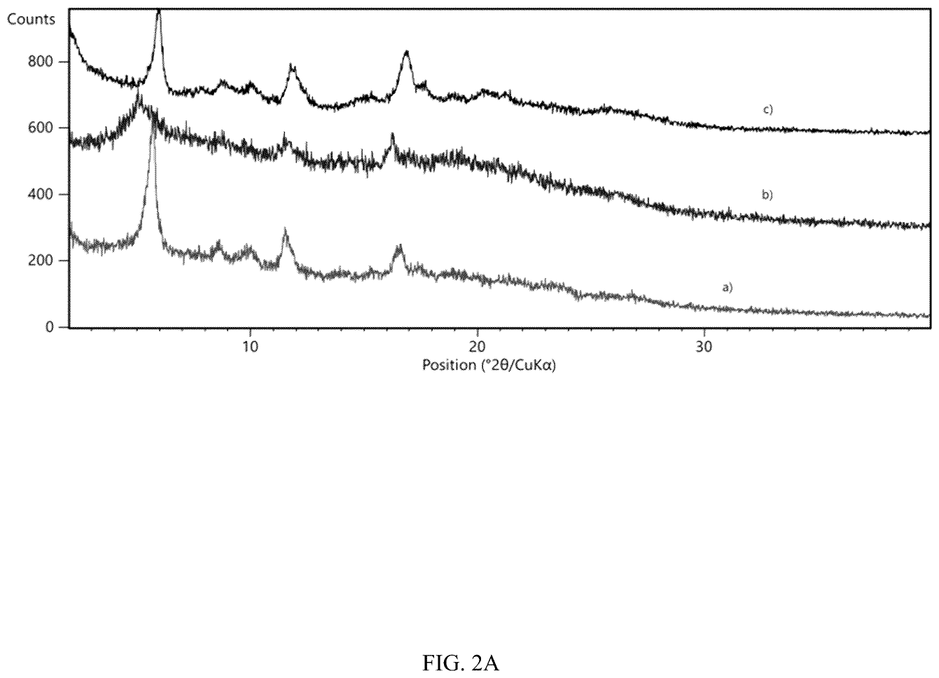

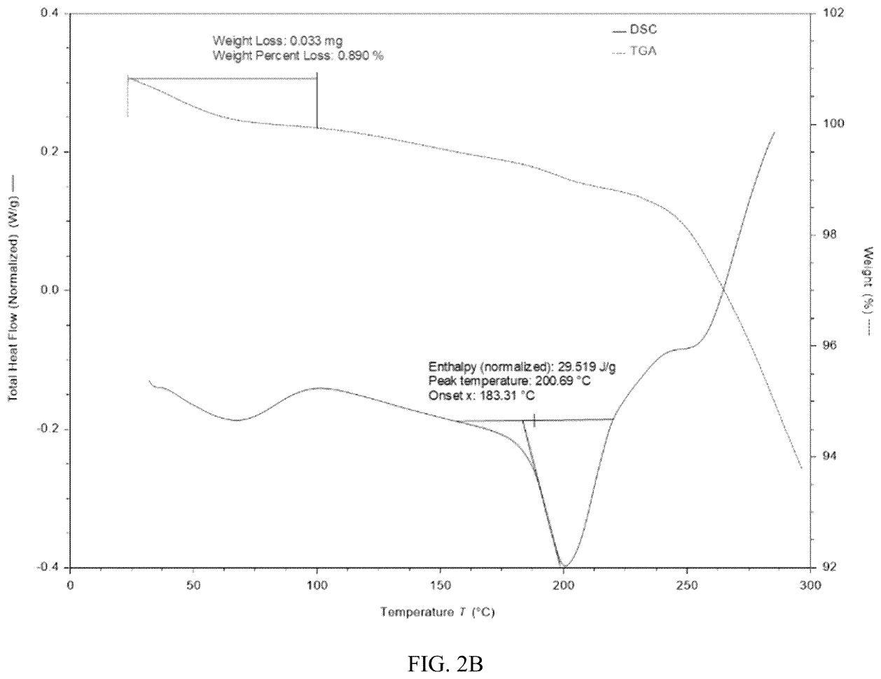

In some embodiments, the present disclosure provides compound 3, wherein said compound has an XRPD substantially similar (e.g., having peaks ±0.2 degree 2-theta) to that depicted in FIG. 2A.

In some embodiments, the present disclosure provides compound 3, wherein said compound has a DSC-TGA substantially similar (e.g., having an endothermic DSC event onset at 180-185° C.) to that depicted in FIG. 2B. In some embodiments, the present disclosure provides compound 3, wherein said compound has a DSC event and/or a TGA weight loss as described in Table 6.

In some embodiments, the present disclosure provides a composition comprising compound 3 and a pharmaceutically acceptable carrier or excipient.

In some embodiments, the present disclosure provides a method of modulating IRAK4 kinase in a patient comprising administering to said patient compound 3 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 3 or a composition thereof. In some embodiments, the present disclosure provides a method of treating a disease, disorder or condition in a patient, comprising administering to said patient compound 3 or a composition thereof, wherein the disease, disorder or condition is mediated by IRAK4. In some embodiments, the present disclosure provides a method of treating an autoimmune or inflammatory disorder in a patient, comprising administering to said patient compound 3 or a composition thereof.

### Compound 4 (Toluenesulfonic Acid Salts of Compound 1)

According to one embodiment, the present disclosure provides a toluenesulfonic acid salt of compound 1, represented by compound 4:

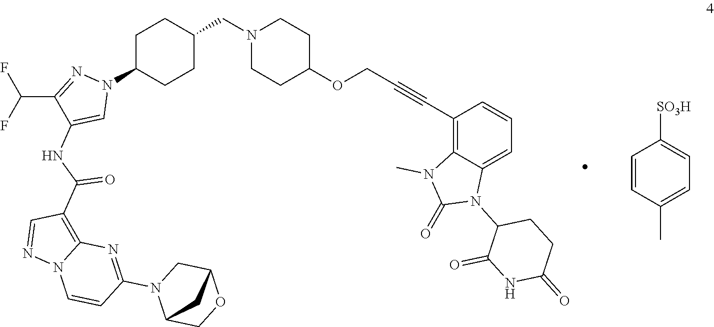 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; Cc1ccc(S(=O)(=O)O)cc1]

It will be appreciated by one of ordinary skill in the art that the toluenesulfonic acid and compound 1 are ionically bonded to form compound 4. In some embodiments, compound 4 is a toluenesulfate salt. It is contemplated that compound 4 can exist in a variety of physical forms. For example, compound 4 can be in solution, suspension, or in solid form. In certain embodiments, compound 4 is in solid form. When compound 4 is in solid form, said compound may be amorphous, crystalline, or a mixture thereof. Exemplary solid forms are described in more detail below.

In some embodiments, the present disclosure provides compound 4 substantially free of impurities. Such impurities or extraneous matter may include different forms of compound 4, residual solvents, or any other impurities that may result from the preparation of, and/or isolation of, compound 4. In certain embodiments, at least about 95% by weight of a form of compound 4 is present. In still other embodiments of the disclosure, at least about 99% by weight of a form of compound 4 is present.

According to one embodiment, a form of compound 4 is present in an amount of at least about 97, 97.5, 98.0, 98.5, 99, 99.5, 99.8 weight percent where the percentages are based on the total weight of the composition. According to another embodiment, a form of compound 4 contains no more than about 3.0 area percent HPLC of total organic impurities and, in certain embodiments, no more than about 1.5 area percent HPLC total organic impurities relative to the total area of the HPLC chromatogram. In other embodiments, a form of compound 4 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 4 is also meant to include all tautomeric forms of compound 4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 4 can exist in a variety of solid forms. Exemplary such forms include polymorphs such as those described herein.

In certain embodiments, compound 4 is a crystalline solid. In other embodiments, compound 4 is a crystalline solid substantially free of amorphous compound 4. As used herein, the term “substantially free of amorphous compound 4” means that the compound contains no significant amount of amorphous compound 4. In certain embodiments, at least about 95% by weight of crystalline compound 4 is present. In still other embodiments of the disclosure, at least about 99% by weight of crystalline compound 4 is present.

It has been found that compound 4 can exist in at least one distinct polymorphic form. In certain embodiments, the present disclosure provides a polymorphic form of compound 4 referred to herein as Form A.

### Form A of Compound 4

Methods for preparing Form A of compound 4 are described infra.

In some embodiments, the disclosure provides compound 4:

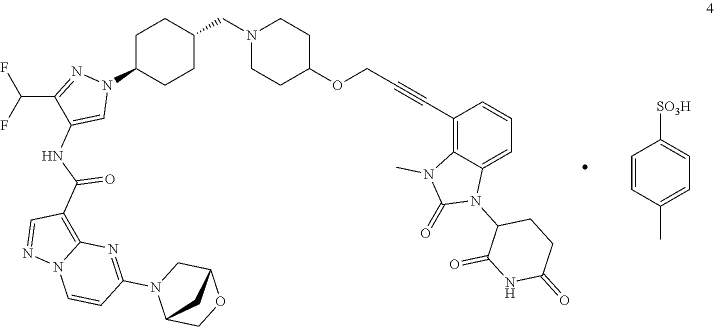 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; Cc1ccc(S(=O)(=O)O)cc1]

wherein said compound is crystalline. In some embodiments, the present disclosure provides compound 4, wherein said compound is substantially free of amorphous compound 4. In some embodiments, the present disclosure provides compound 4, wherein said compound is substantially free of amorphous compound 1. In some embodiments, the present disclosure provides compound 4, wherein said compound is substantially free of a crystalline form of compound 1. In some embodiments, the present disclosure provides compound 4, wherein said compound is substantially free of impurities.

In some embodiments, Form A of compound 4 is characterized by one or more peaks in its XRPD pattern selected from those at 5.9, 11.9, and 16.5±0.5 degree 2-theta. In some embodiments, Form A of compound 4 is characterized by one or more peaks in its XRPD pattern selected from those at 5.9, 11.9, and 16.5±0.4 degree 2-theta. In some embodiments, Form A of compound 4 is characterized by one or more peaks in its XRPD pattern selected from those at 5.9, 11.9, and 16.5±0.3 degree 2-theta. In some embodiments, Form A of compound 4 is characterized by one or more peaks in its XRPD pattern selected from those at 5.9, 11.9, and 16.5±0.2 degree 2-theta.

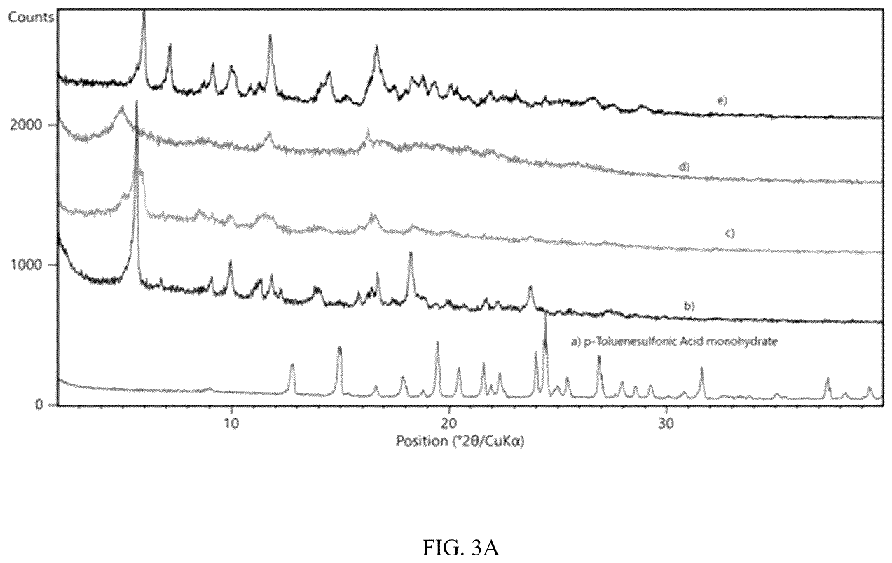

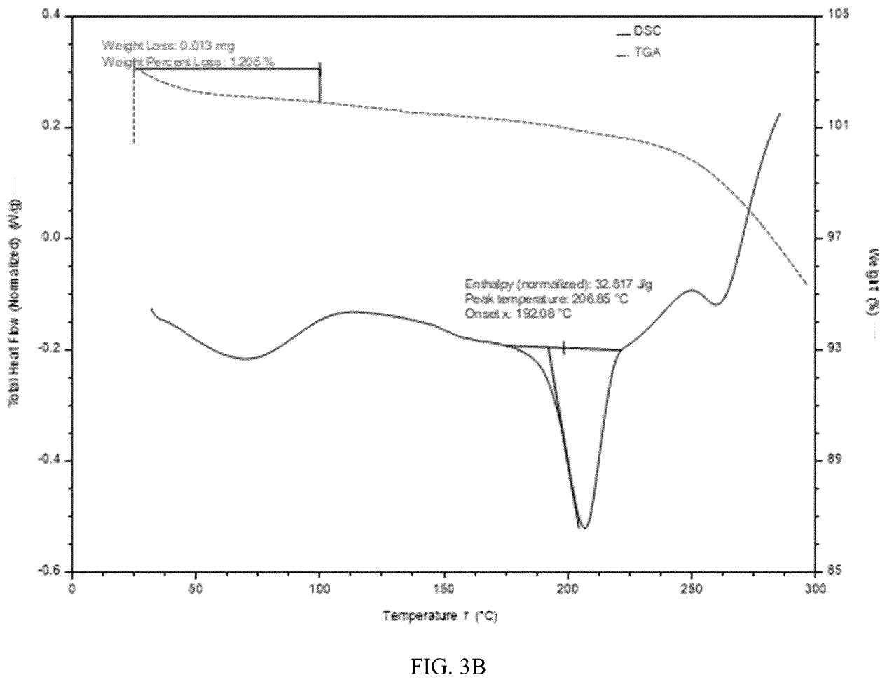

In some embodiments, the present disclosure provides compound 4, wherein said compound has an XRPD substantially similar (e.g., having peaks ±0.2 degree 2-theta) to that depicted in FIG. 3A.

In some embodiments, the present disclosure provides a composition comprising compound 4 and a pharmaceutically acceptable carrier or excipient.

In some embodiments, the present disclosure provides compound 4, wherein said compound has a DSC-TGA substantially similar (e.g., having an endothermic DSC event onset at 190-195° C.) to that depicted in FIG. 3B. In some embodiments, the present disclosure provides compound 4, wherein said compound has a DSC event and/or a TGA weight loss as described in Table 6.

In some embodiments, the present disclosure provides a method of modulating IRAK4 kinase in a patient comprising administering to said patient compound 4 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 4 or a composition thereof. In some embodiments, the present disclosure provides a method of treating a disease, disorder or condition in a patient, comprising administering to said patient compound 4 or a composition thereof, wherein the disease, disorder or condition is mediated by IRAK4. In some embodiments, the present disclosure provides a method of treating an autoimmune or inflammatory disorder in a patient, comprising administering to said patient compound 4 or a composition thereof.

### Compound 5 (Ethanesulfonic Acid Salts of Compound 1)

According to one embodiment, the present disclosure provides an ethanesulfonic acid salt of compound 1, represented by compound 5:

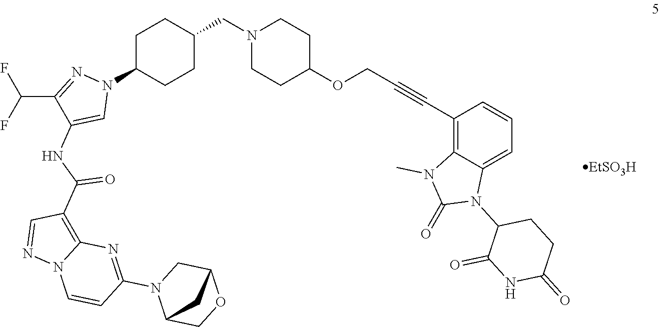 [SMILES: "·EtSO3H" ; Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

It will be appreciated by one of ordinary skill in the art that the ethanesulfonic acid and compound 1 are ionically bonded to form compound 5. In some embodiments, compound 5 is an ethanesulfate salt. It is contemplated that compound 5 can exist in a variety of physical forms. For example, compound 5 can be in solution, suspension, or in solid form. In certain embodiments, compound 5 is in solid form. When compound 5 is in solid form, said compound may be amorphous, crystalline, or a mixture thereof. Exemplary solid forms are described in more detail below.

In some embodiments, the present disclosure provides compound 5 substantially free of impurities. Such impurities or extraneous matter may include different forms of compound 5, residual solvents, or any other impurities that may result from the preparation of, and/or isolation of, compound 5. In certain embodiments, at least about 95% by weight of a form of compound 5 is present. In still other embodiments of the disclosure, at least about 99% by weight of a form of compound 5 is present.

According to one embodiment, a form of compound 5 is present in an amount of at least about 97, 97.5, 98.0, 98.5, 99, 99.5, 99.8 weight percent where the percentages are based on the total weight of the composition. According to another embodiment, a form of compound 5 contains no more than about 3.0 area percent HPLC of total organic impurities and, in certain embodiments, no more than about 1.5 area percent HPLC total organic impurities relative to the total area of the HPLC chromatogram. In other embodiments, a form of compound 5 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 5 is also meant to include all tautomeric forms of compound 5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 5 can exist in a variety of solid forms. Exemplary such forms include polymorphs such as those described herein.

In certain embodiments, compound 5 is a crystalline solid. In other embodiments, compound 5 is a crystalline solid substantially free of amorphous compound 5. As used herein, the term “substantially free of amorphous compound 5” means that the compound contains no significant amount of amorphous compound 5. In certain embodiments, at least about 95% by weight of crystalline compound 5 is present. In still other embodiments of the disclosure, at least about 99% by weight of crystalline compound 5 is present.

It has been found that compound 5 can exist in at least one distinct polymorphic form. In certain embodiments, the present disclosure provides a polymorphic form of compound 5 referred to herein as Form A.

### Form A of Compound 5

Methods for preparing Form A of compound 5 are described infra.

In some embodiments, the disclosure provides compound 5:

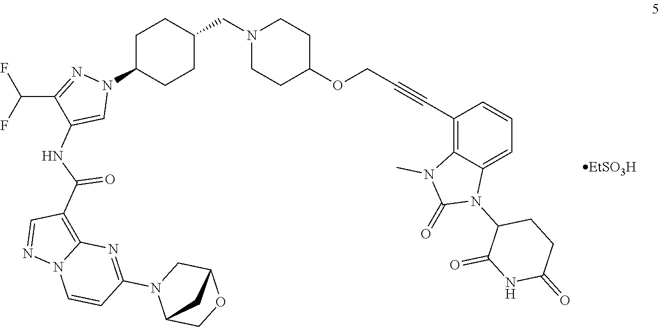 [SMILES: "·EtSO3H" ; Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

wherein said compound is crystalline. In some embodiments, the present disclosure provides compound 5, wherein said compound is substantially free of amorphous compound 5. In some embodiments, the present disclosure provides compound 5, wherein said compound is substantially free of amorphous compound 1. In some embodiments, the present disclosure provides compound 5, wherein said compound is substantially free of a crystalline form of compound 1. In some embodiments, the present disclosure provides compound 5, wherein said compound is substantially free of impurities.

In some embodiments, Form A of compound 5 is characterized by one or more peaks in its XRPD pattern selected from those at 6.0, 8.2, and 12.4±0.5 degree 2-theta. In some embodiments, Form A of compound 5 is characterized by one or more peaks in its XRPD pattern selected from those at 6.0, 8.2, and 12.4±0.4 degree 2-theta. In some embodiments, Form A of compound 5 is characterized by one or more peaks in its XRPD pattern selected from those at 6.0, 8.2, and 12.4±0.3 degree 2-theta. In some embodiments, Form A of compound 5 is characterized by one or more peaks in its XRPD pattern selected from those at 6.0, 8.2, and 12.4±0.2 degree 2-theta.

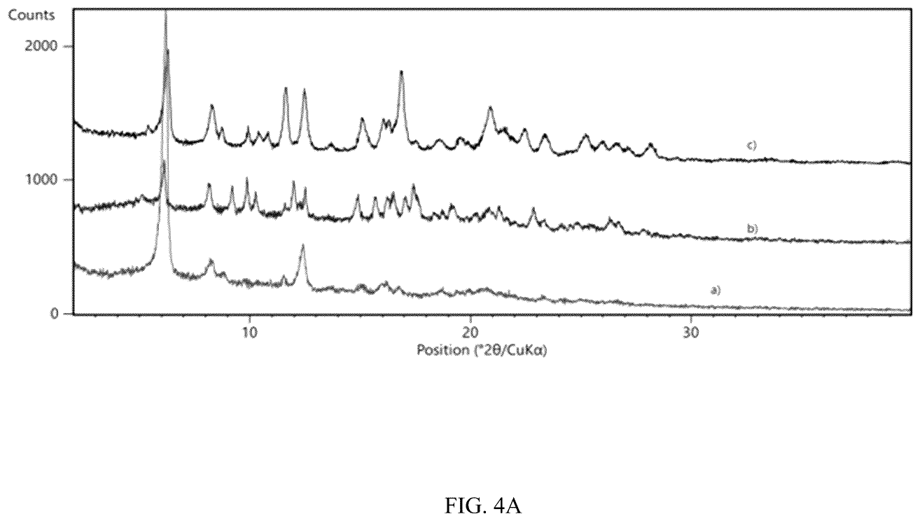

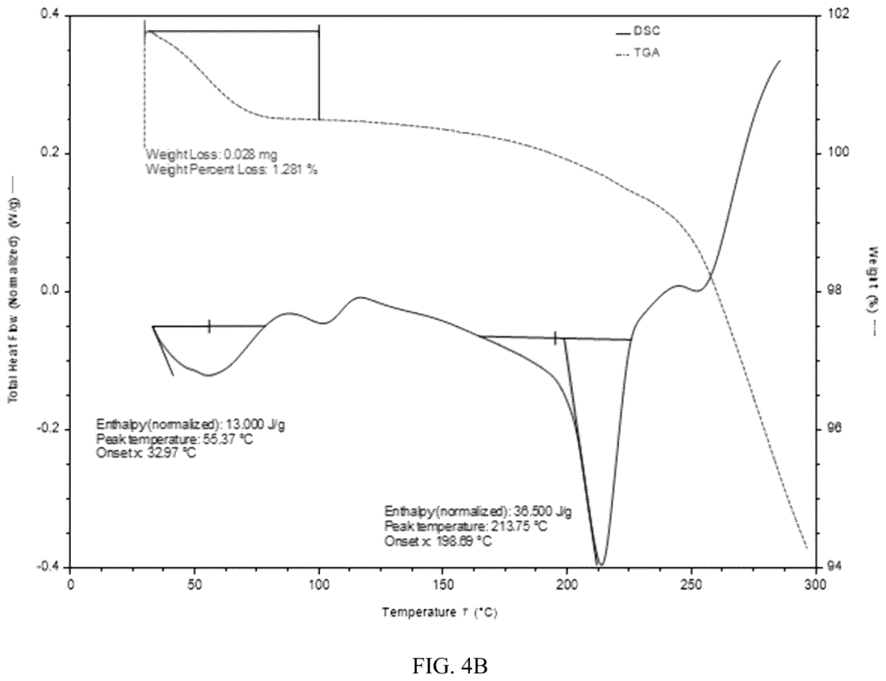

In some embodiments, the present disclosure provides compound 5, wherein said compound has an XRPD substantially similar (e.g., having peaks ±0.2 degree 2-theta) to that depicted in FIG. 4A.

In some embodiments, the present disclosure provides a composition comprising compound 5 and a pharmaceutically acceptable carrier or excipient.

In some embodiments, the present disclosure provides compound 5, wherein said compound has a DSC-TGA substantially similar (e.g., having an endothermic DSC event onset at 195-200° C.) to that depicted in FIG. 4B. In some embodiments, the present disclosure provides compound 5, wherein said compound has a DSC event and/or a TGA weight loss as described in Table 6.

In some embodiments, the present disclosure provides a method of modulating IRAK4 kinase in a patient comprising administering to said patient compound 5 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 5 or a composition thereof. In some embodiments, the present disclosure provides a method of treating a disease, disorder or condition in a patient, comprising administering to said patient compound 5 or a composition thereof, wherein the disease, disorder or condition is mediated by IRAK4. In some embodiments, the present disclosure provides a method of treating an autoimmune or inflammatory disorder in a patient, comprising administering to said patient compound 5 or a composition thereof.

### Compound 6 (Methanesulfonic Acid Salts of Compound 1)

According to one embodiment, the present disclosure provides a methanesulfonic acid salt of compound 1, represented by compound 6:

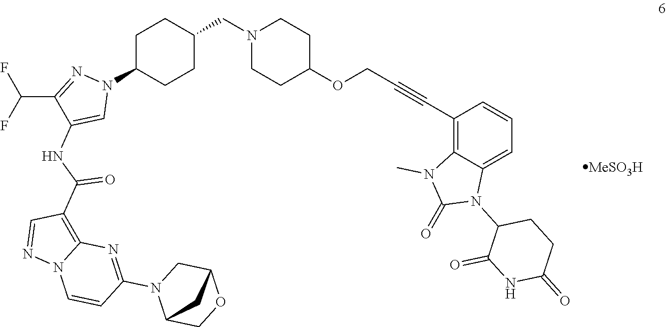 [SMILES: "·MeSO3H" ; Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

It will be appreciated by one of ordinary skill in the art that the methanesulfonic acid and compound 1 are ionically bonded to form compound 6. In some embodiments, compound 6 is a methanesulfate salt. It is contemplated that compound 6 can exist in a variety of physical forms. For example, compound 6 can be in solution, suspension, or in solid form. In certain embodiments, compound 6 is in solid form. When compound 6 is in solid form, said compound may be amorphous, crystalline, or a mixture thereof. Exemplary solid forms are described in more detail below.

In some embodiments, the present disclosure provides compound 6 substantially free of impurities. Such impurities or extraneous matter may include different forms of compound 6, residual solvents, or any other impurities that may result from the preparation of, and/or isolation of, compound 6. In certain embodiments, at least about 95% by weight of a form of compound 6 is present. In still other embodiments of the disclosure, at least about 99% by weight of a form of compound 6 is present.

According to one embodiment, a form of compound 6 is present in an amount of at least about 97, 97.5, 98.0, 98.5, 99, 99.5, 99.8 weight percent where the percentages are based on the total weight of the composition. According to another embodiment, a form of compound 6 contains no more than about 3.0 area percent HPLC of total organic impurities and, in certain embodiments, no more than about 1.5 area percent HPLC total organic impurities relative to the total area of the HPLC chromatogram. In other embodiments, a form of compound 6 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 6 is also meant to include all tautomeric forms of compound 6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 6 can exist in a variety of solid forms. Exemplary such forms include polymorphs such as those described herein.

In certain embodiments, compound 6 is a crystalline solid. In other embodiments, compound 6 is a crystalline solid substantially free of amorphous compound 6. As used herein, the term “substantially free of amorphous compound 6” means that the compound contains no significant amount of amorphous compound 6. In certain embodiments, at least about 95% by weight of crystalline compound 6 is present. In still other embodiments of the disclosure, at least about 99% by weight of crystalline compound 6 is present.

It has been found that compound 6 can exist in at least one distinct polymorphic form. In certain embodiments, the present disclosure provides a polymorphic form of compound 6 referred to herein as Form A.

### Form A of Compound 6

Methods for preparing Form A of compound 6 are described infra.

In some embodiments, the disclosure provides compound 6:

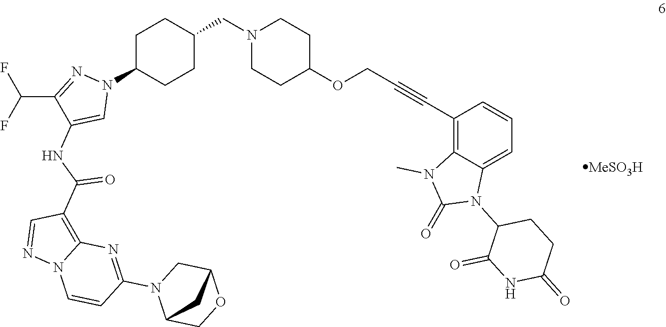 [SMILES: "·MeSO3H" ; Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

wherein said compound is crystalline. In some embodiments, the present disclosure provides compound 6, wherein said compound is substantially free of amorphous compound 6. In some embodiments, the present disclosure provides compound 6, wherein said compound is substantially free of amorphous compound 1. In some embodiments, the present disclosure provides compound 6, wherein said compound is substantially free of a crystalline form of compound 1. In some embodiments, the present disclosure provides compound 6, wherein said compound is substantially free of impurities.

In some embodiments, Form A of compound 6 is characterized by one or more peaks in its XRPD pattern selected from those at 16.5, 19.5, and 21.2±0.5 degree 2-theta. In some embodiments, Form A of compound 6 is characterized by one or more peaks in its XRPD pattern selected from those at 16.5, 19.5, and 21.2±0.4 degree 2-theta. In some embodiments, Form A of compound 6 is characterized by one or more peaks in its XRPD pattern selected from those at 16.5, 19.5, and 21.2±0.3 degree 2-theta. In some embodiments, Form A of compound 6 is characterized by one or more peaks in its XRPD pattern selected from those at 3.9, 11.9, and 16.5±0.2 degree 2-theta.

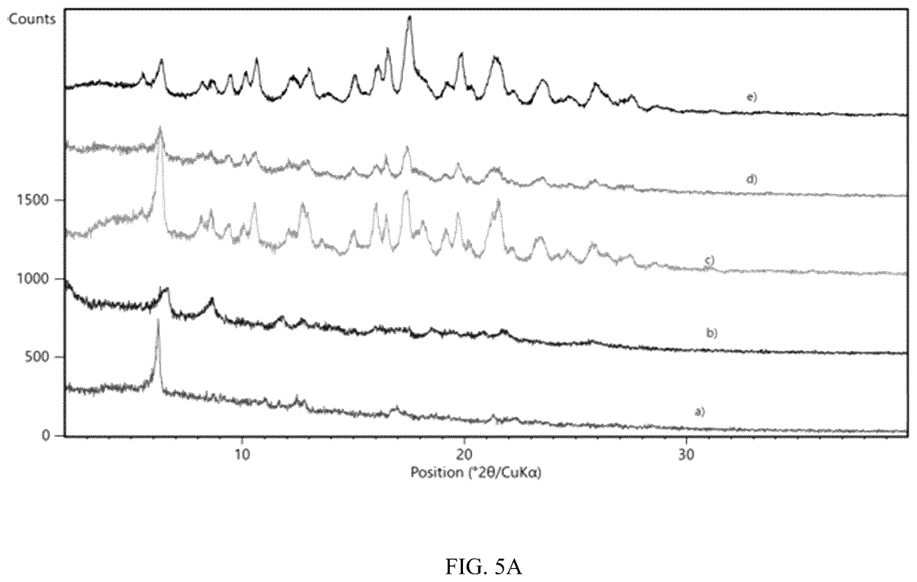

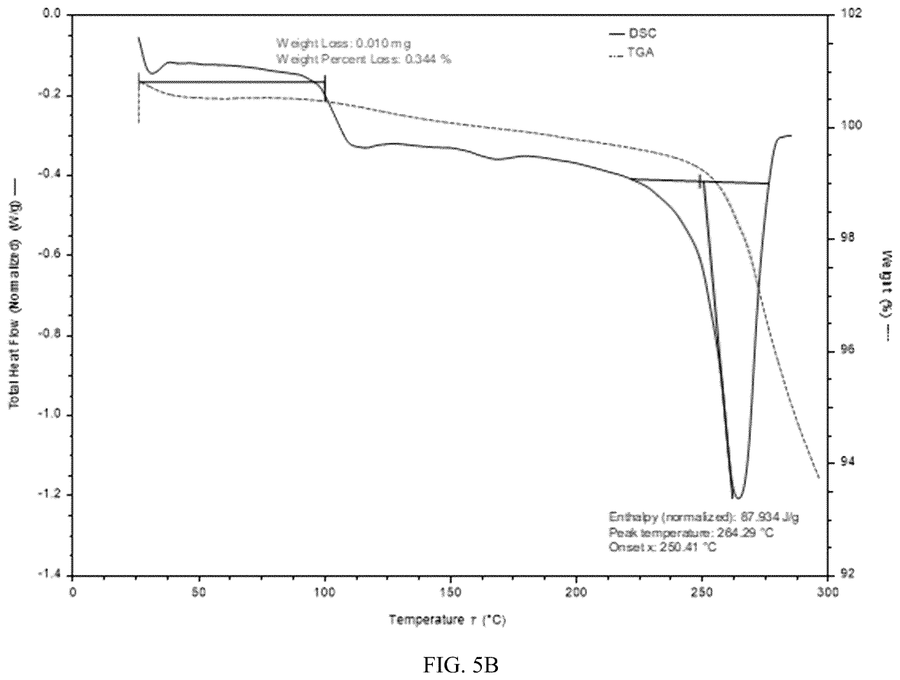

In some embodiments, the present disclosure provides compound 6, wherein said compound has an XRPD substantially similar (e.g., having peaks ±0.2 degree 2-theta) to that depicted in FIG. 5A.

In some embodiments, the present disclosure provides a composition comprising compound 6 and a pharmaceutically acceptable carrier or excipient.

In some embodiments, the present disclosure provides compound 6, wherein said compound has a DSC-TGA substantially similar (e.g., having an endothermic DSC onset event at 248-253° C.) to that depicted in FIG. 5B. In some embodiments, the present disclosure provides compound 6, wherein said compound has a DSC event and/or a TGA weight loss as described in Table 6.

In some embodiments, the present disclosure provides a method of modulating IRAK4 kinase in a patient comprising administering to said patient compound 6 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 6 or a composition thereof. In some embodiments, the present disclosure provides a method of treating a disease, disorder or condition in a patient, comprising administering to said patient compound 6 or a composition thereof, wherein the disease, disorder or condition is mediated by IRAK4. In some embodiments, the present disclosure provides a method of treating an autoimmune or inflammatory disorder in a patient, comprising administering to said patient compound 6 or a composition thereof.

### Compound 7 (Maleic Acid Salts of Compound 1)

According to one embodiment, the present disclosure provides a maleic acid salt of compound 1, represented by compound 7:

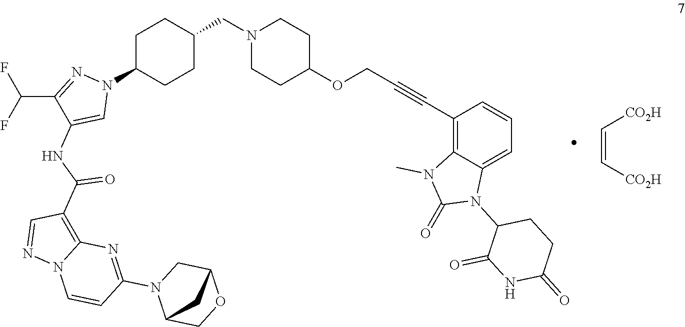 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)/C=C\C(=O)O]

It will be appreciated by one of ordinary skill in the art that the maleic acid and compound 1 are ionically bonded to form compound 7. In some embodiments, compound 7 is a maleate salt. In some embodiments, compound 7 is a hydrogen maleate salt. In some embodiments, compound 7 exists as a mixture of hydrogen maleate and maleate salts. It is contemplated that compound 7 can exist in a variety of physical forms. For example, compound 7 can be in solution, suspension, or in solid form. In certain embodiments, compound 7 is in solid form. When compound 7 is in solid form, said compound may be amorphous, crystalline, or a mixture thereof. Exemplary solid forms are described in more detail below.

In some embodiments, the present disclosure provides compound 7 substantially free of impurities. Such impurities or extraneous matter may include different forms of compound 7, residual solvents, or any other impurities that may result from the preparation of, and/or isolation of, compound 7. In certain embodiments, at least about 95% by weight of a form of compound 7 is present. In still other embodiments of the disclosure, at least about 99% by weight of a form of compound 7 is present.

According to one embodiment, a form of compound 7 is present in an amount of at least about 97, 97.5, 98.0, 98.5, 99, 99.5, 99.8 weight percent where the percentages are based on the total weight of the composition. According to another embodiment, a form of compound 7 contains no more than about 3.0 area percent HPLC of total organic impurities and, in certain embodiments, no more than about 1.5 area percent HPLC total organic impurities relative to the total area of the HPLC chromatogram. In other embodiments, a form of compound 7 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 7 is also meant to include all tautomeric forms of compound 7. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 7 can exist in a variety of solid forms. Exemplary such forms include polymorphs such as those described herein.

In certain embodiments, compound 7 is a crystalline solid. In other embodiments, compound 7 is a crystalline solid substantially free of amorphous compound 7. As used herein, the term “substantially free of amorphous compound 7” means that the compound contains no significant amount of amorphous compound 7. In certain embodiments, at least about 95% by weight of crystalline compound 7 is present. In still other embodiments of the disclosure, at least about 99% by weight of crystalline compound 7 is present.

It has been found that compound 7 can exist in at least one distinct polymorphic form. In certain embodiments, the present disclosure provides a polymorphic form of compound 7 referred to herein as Form A.

### Form A of Compound 7

Methods for preparing Form A of compound 7 are described infra.

In some embodiments, the disclosure provides compound 7:

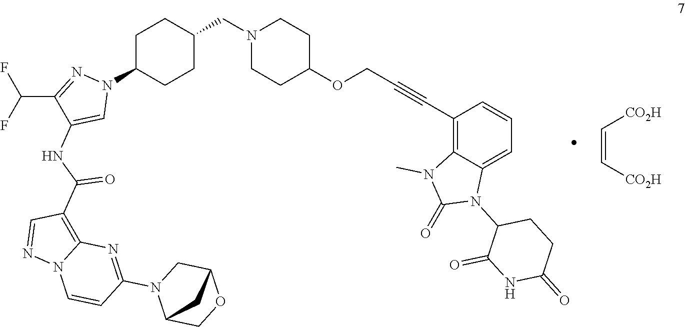 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)/C=C\C(=O)O]

wherein said compound is crystalline. In some embodiments, the present disclosure provides compound 7, wherein said compound is substantially free of amorphous compound 7. In some embodiments, the present disclosure provides compound 7, wherein said compound is substantially free of amorphous compound 1. In some embodiments, the present disclosure provides compound 7, wherein said compound is substantially free of a crystalline form of compound 1. In some embodiments, the present disclosure provides compound 7, wherein said compound is substantially free of impurities.

In some embodiments, Form A of compound 7 is characterized by one or more peaks in its XRPD pattern selected from those at 6.5, 16.5, and 18.5±0.5 degree 2-theta. In some embodiments, Form A of compound 7 is characterized by one or more peaks in its XRPD pattern selected from those at 6.5, 16.5, and 18.5±0.4 degree 2-theta. In some embodiments, Form A of compound 7 is characterized by one or more peaks in its XRPD pattern selected from those at 6.5, 16.5, and 18.5±0.3 degree 2-theta. In some embodiments, Form A of compound 7 is characterized by one or more peaks in its XRPD pattern selected from those at 6.5, 16.5, and 18.5±0.2 degree 2-theta.

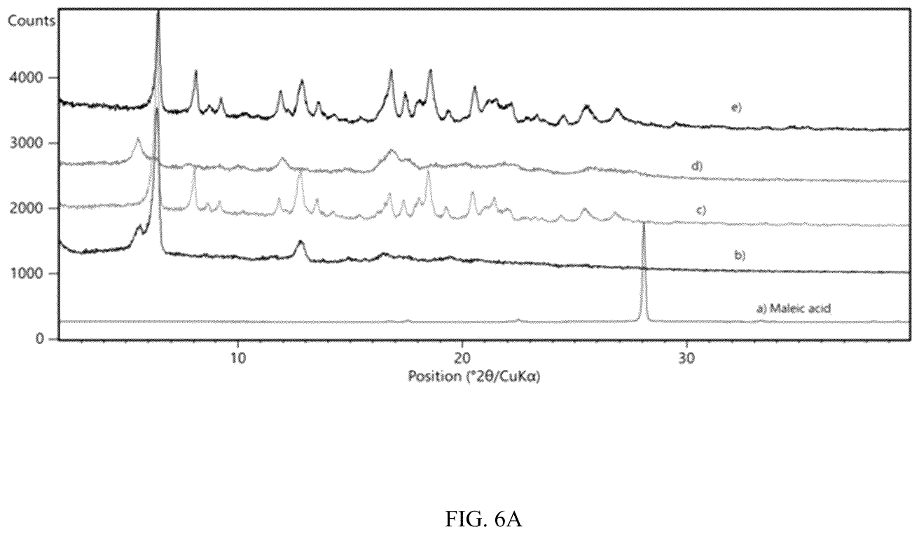

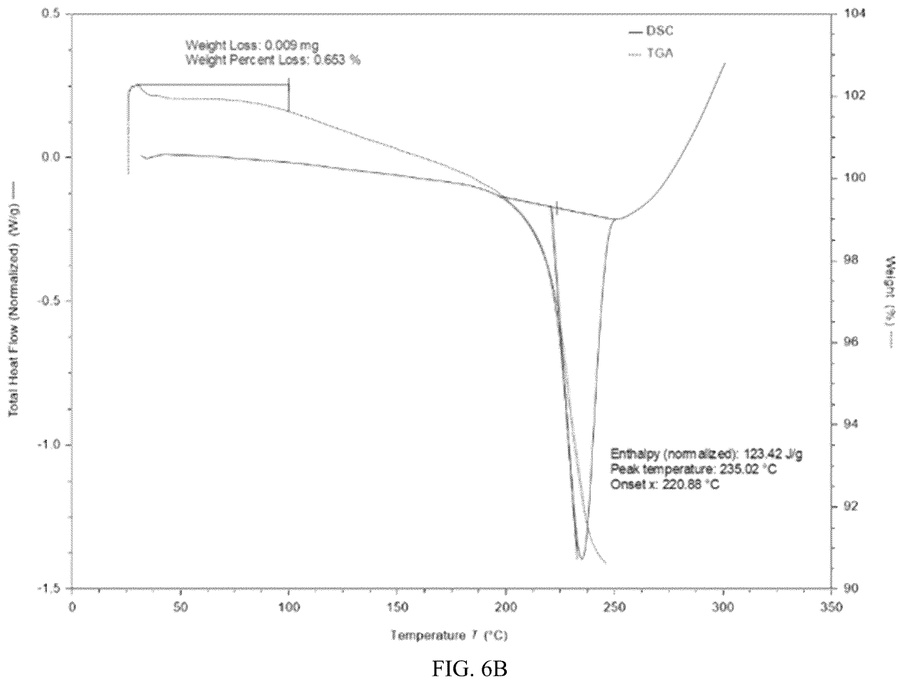

In some embodiments, the present disclosure provides compound 7, wherein said compound has an XRPD substantially similar (e.g., having peaks ±0.2 degree 2-theta) to that depicted in FIG. 6A.

In some embodiments, the present disclosure provides a composition comprising compound 7 and a pharmaceutically acceptable carrier or excipient.

In some embodiments, the present disclosure provides compound 7, wherein said compound has a DSC-TGA substantially similar (e.g., having an endothermic DSC event onset at 218-223° C.) to that depicted in FIG. 6B. In some embodiments, the present disclosure provides compound 7, wherein said compound has a DSC event and/or a TGA weight loss as described in Table 6.

In some embodiments, the present disclosure provides a method of modulating IRAK4 kinase in a patient comprising administering to said patient compound 7 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 7 or a composition thereof. In some embodiments, the present disclosure provides a method of treating a disease, disorder or condition in a patient, comprising administering to said patient compound 7 or a composition thereof, wherein the disease, disorder or condition is mediated by IRAK4. In some embodiments, the present disclosure provides a method of treating an autoimmune or inflammatory disorder in a patient, comprising administering to said patient compound 7 or a composition thereof.

### Compound 8 (L-Tartaric Acid Salts of Compound 1)

According to one embodiment, the present disclosure provides a L-tartaric acid salt of compound 1, represented by compound 8:

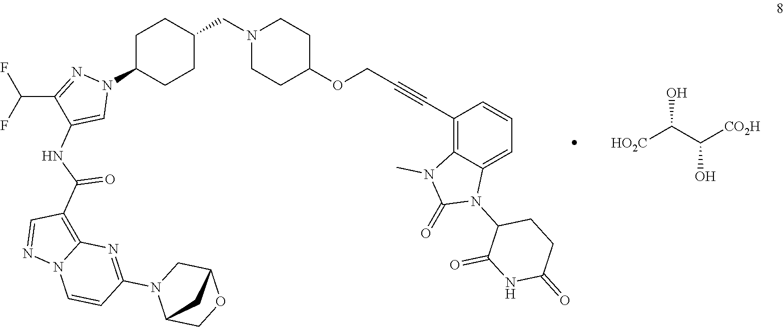 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)[C@H](O)[C@@H](O)C(=O)O]

It will be appreciated by one of ordinary skill in the art that the L-tartaric acid and compound 1 are ionically bonded to form compound 8. In some embodiments, compound 8 is a hydrogen L-tartrate salt. In some embodiments, compound 8 is a L-tartrate salt. In some embodiments, compound 8 exists as a mixture of hydrogen L-tartrate and L-tartrate salts. It is contemplated that compound 8 can exist in a variety of physical forms. For example, compound 8 can be in solution, suspension, or in solid form. In certain embodiments, compound 8 is in solid form. When compound 8 is in solid form, said compound may be amorphous, crystalline, or a mixture thereof. Exemplary solid forms are described in more detail below.

In some embodiments, the present disclosure provides compound 8 substantially free of impurities. Such impurities or extraneous matter may include different forms of compound 8, residual solvents, or any other impurities that may result from the preparation of, and/or isolation of, compound 8. In certain embodiments, at least about 95% by weight of a form of compound 8 is present. In still other embodiments of the disclosure, at least about 99% by weight of a form of compound 8 is present.

According to one embodiment, a form of compound 8 is present in an amount of at least about 97, 97.5, 98.0, 98.5, 99, 99.5, 99.8 weight percent where the percentages are based on the total weight of the composition. According to another embodiment, a form of compound 8 contains no more than about 3.0 area percent HPLC of total organic impurities and, in certain embodiments, no more than about 1.5 area percent HPLC total organic impurities relative to the total area of the HPLC chromatogram. In other embodiments, a form of compound 8 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 8 is also meant to include all tautomeric forms of compound 8. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 8 can exist in a variety of solid forms. Exemplary such forms include polymorphs such as those described herein.

In certain embodiments, compound 8 is a crystalline solid. In other embodiments, compound 8 is a crystalline solid substantially free of amorphous compound 8. As used herein, the term “substantially free of amorphous compound 8” means that the compound contains no significant amount of amorphous compound 8. In certain embodiments, at least about 95% by weight of crystalline compound 9 is present. In still other embodiments of the disclosure, at least about 99% by weight of crystalline compound 8 is present.

It has been found that compound 8 can exist in at least one distinct polymorphic form. In certain embodiments, the present disclosure provides a polymorphic form of compound 8 referred to herein as Form A.

### Form A of Compound 8

Methods for preparing Form A of compound 8 are described infra.

In some embodiments, the disclosure provides compound 8:

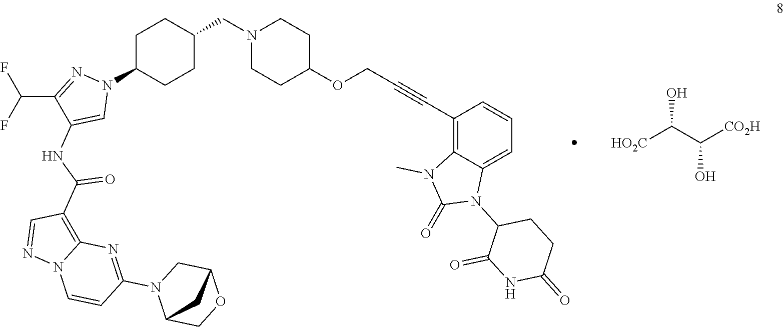 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)[C@H](O)[C@@H](O)C(=O)O]

wherein said compound is crystalline. In some embodiments, the present disclosure provides compound 8, wherein said compound is substantially free of amorphous compound 8. In some embodiments, the present disclosure provides compound 8, wherein said compound is substantially free of amorphous compound 1. In some embodiments, the present disclosure provides compound 8, wherein said compound is substantially free of a crystalline form of compound 1. In some embodiments, the present disclosure provides compound 9, wherein said compound is substantially free of impurities.

In some embodiments, Form A of compound 8 is characterized by one or more peaks in its XRPD pattern selected from those at 6.5, 16.5, and 17.5±0.5 degree 2-theta. In some embodiments, Form A of compound 8 is characterized by one or more peaks in its XRPD pattern selected from those at 6.5, 16.5, and 17.5±0.4 degree 2-theta. In some embodiments, Form A of compound 8 is characterized by one or more peaks in its XRPD pattern selected from those at 6.5, 16.5, and 17.5±0.3 degree 2-theta. In some embodiments, Form A of compound 8 is characterized by one or more peaks in its XRPD pattern selected from those at 6.5, 16.5, and 17.5±0.2 degree 2-theta.

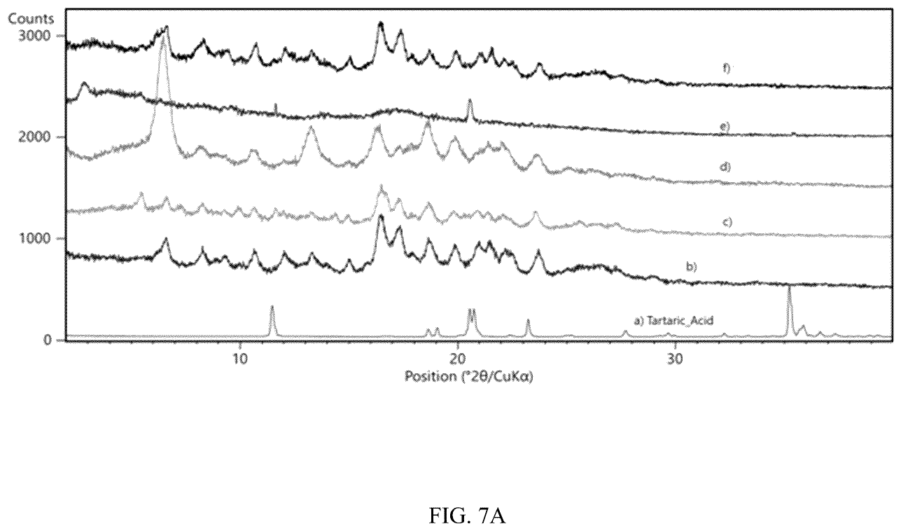

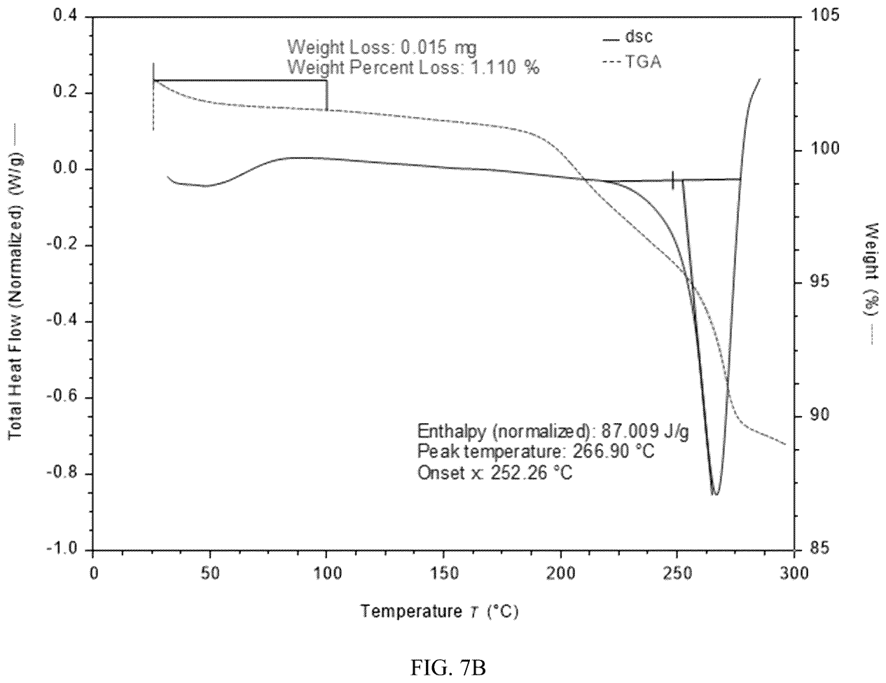

In some embodiments, the present disclosure provides compound 8, wherein said compound has an XRPD substantially similar (e.g., having peaks ±0.2 degree 2-theta) to that depicted in FIG. 7A.

In some embodiments, the present disclosure provides a composition comprising compound 8 and a pharmaceutically acceptable carrier or excipient.

In some embodiments, the present disclosure provides compound 8, wherein said compound has a DSC-TGA substantially similar (e.g., having an endothermic DSC event onset at 250-255° C.) to that depicted in FIG. 7B. In some embodiments, the present disclosure provides compound 8, wherein said compound has a DSC event and/or a TGA weight loss as described in Table 6.

In some embodiments, the present disclosure provides a method of modulating IRAK4 kinase in a patient comprising administering to said patient compound 8 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 8 or a composition thereof. In some embodiments, the present disclosure provides a method of treating a disease, disorder or condition in a patient, comprising administering to said patient compound 8 or a composition thereof, wherein the disease, disorder or condition is mediated by IRAK4. In some embodiments, the present disclosure provides a method of treating an autoimmune or inflammatory disorder in a patient, comprising administering to said patient compound 9 or a composition thereof.

### Compound 9 (Fumaric Acid Salts of Compound 1)

According to one embodiment, the present disclosure provides a fumaric acid salt of compound 1, represented by compound 9:

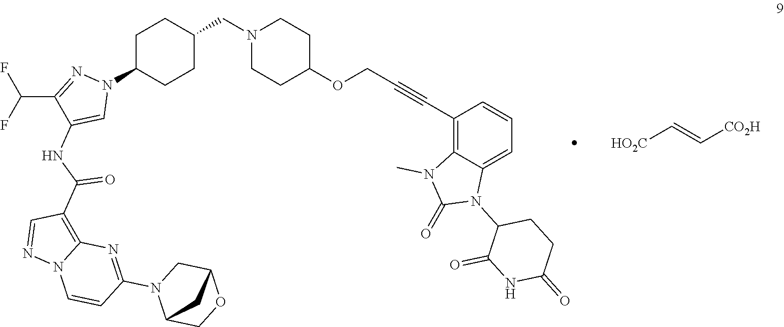 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)/C=C/C(=O)O]

It will be appreciated by one of ordinary skill in the art that the fumaric acid and compound 1 are ionically bonded to form compound 9. In some embodiments, compound 9 is a hydrogen fumarate salt. In some embodiments, compound 9 is a fumarate salt. In some embodiments, compound 9 exists as a mixture of hydrogen fumarate and fumarate salts. It is contemplated that compound 9 can exist in a variety of physical forms. For example, compound 9 can be in solution, suspension, or in solid form. In certain embodiments, compound 9 is in solid form. When compound 9 is in solid form, said compound may be amorphous, crystalline, or a mixture thereof. Exemplary solid forms are described in more detail below.

In some embodiments, the present disclosure provides compound 9 substantially free of impurities. Such impurities or extraneous matter may include different forms of compound 9, residual solvents, or any other impurities that may result from the preparation of, and/or isolation of, compound 9. In certain embodiments, at least about 95% by weight of a form of compound 9 is present. In still other embodiments of the disclosure, at least about 99% by weight of a form of compound 9 is present.

According to one embodiment, a form of compound 9 is present in an amount of at least about 97, 97.5, 98.0, 98.5, 99, 99.5, 99.8 weight percent where the percentages are based on the total weight of the composition. According to another embodiment, a form of compound 9 contains no more than about 3.0 area percent HPLC of total organic impurities and, in certain embodiments, no more than about 1.5 area percent HPLC total organic impurities relative to the total area of the HPLC chromatogram. In other embodiments, a form of compound 9 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 9 is also meant to include all tautomeric forms of compound 9. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 9 can exist in a variety of solid forms. Exemplary such forms include polymorphs such as those described herein.

In certain embodiments, compound 9 is a crystalline solid. In other embodiments, compound 10 is a crystalline solid substantially free of amorphous compound 9. As used herein, the term “substantially free of amorphous compound 9” means that the compound contains no significant amount of amorphous compound 9. In certain embodiments, at least about 95% by weight of crystalline compound 9 is present. In still other embodiments of the disclosure, at least about 99% by weight of crystalline compound 9 is present.

It has been found that compound 9 can exist in at least one distinct polymorphic form. In certain embodiments, the present disclosure provides a polymorphic form of compound 9 referred to herein as Form A.

### Form A of Compound 9

Methods for preparing Form A of compound 9 are described infra.

In some embodiments, the disclosure provides compound 9:

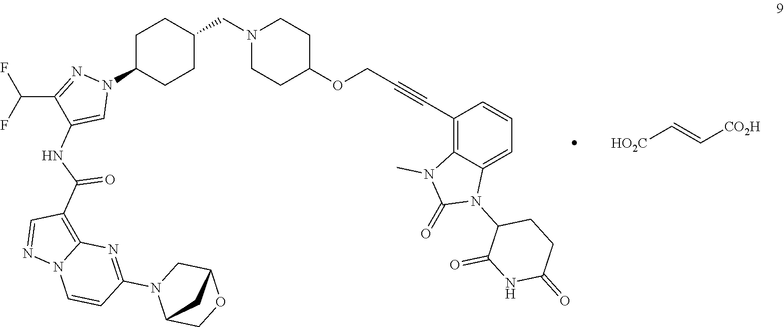 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)/C=C/C(=O)O]

wherein said compound is crystalline. In some embodiments, the present disclosure provides compound 10, wherein said compound is substantially free of amorphous compound 9. In some embodiments, the present disclosure provides compound 9, wherein said compound is substantially free of amorphous compound 1. In some embodiments, the present disclosure provides compound 9, wherein said compound is substantially free of a crystalline form of compound 1. In some embodiments, the present disclosure provides compound 9, wherein said compound is substantially free of impurities.

In some embodiments, Form A of compound 9 is characterized by one or more peaks in its XRPD pattern selected from those at 6.7, 10.7, and 16.3±0.5 degree 2-theta. In some embodiments, Form A of compound 9 is characterized by one or more peaks in its XRPD pattern selected from those at 6.7, 10.7, and 16.3±0.4 degree 2-theta. In some embodiments, Form A of compound 9 is characterized by one or more peaks in its XRPD pattern selected from those at 6.7, 10.7, and 16.3±0.3 degree 2-theta. In some embodiments, Form A of compound 9 is characterized by one or more peaks in its XRPD pattern selected from those at 6.7, 10.7, and 16.3±0.2 degree 2-theta.

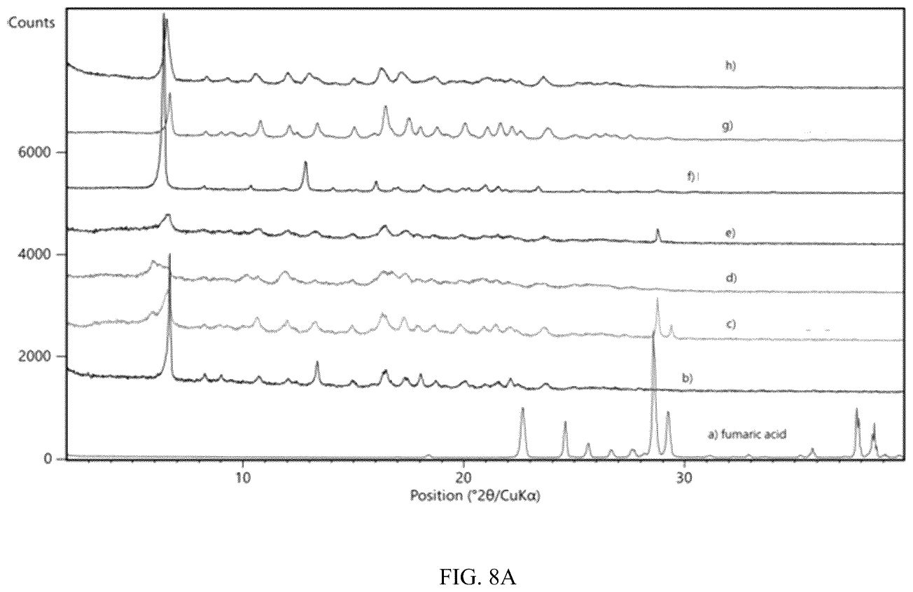

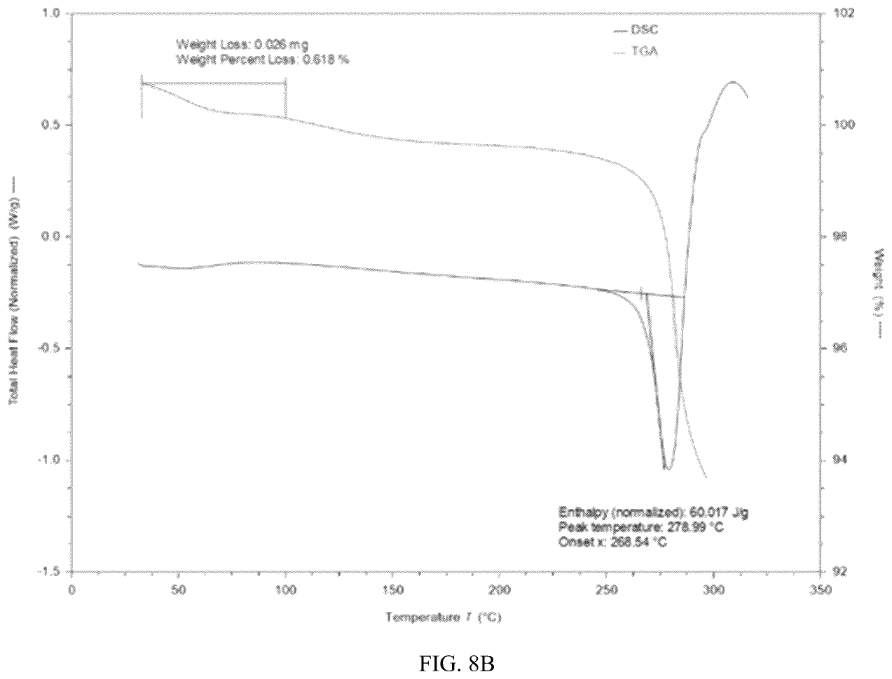

In some embodiments, the present disclosure provides compound 9, wherein said compound has an XRPD substantially similar (e.g., having peaks ±0.2 degree 2-theta) to that depicted in FIG. 8A.

In some embodiments, the present disclosure provides a composition comprising compound 9 and a pharmaceutically acceptable carrier or excipient.

In some embodiments, the present disclosure provides compound 9, wherein said compound has a DSC-TGA substantially similar (e.g., having an endothermic DSC event onset at 266-271° C.) to that depicted in FIG. 8B. In some embodiments, the present disclosure provides compound 9, wherein said compound has a DSC event and/or a TGA weight loss as described in Table 6.

In some embodiments, the present disclosure provides a method of modulating IRAK4 kinase in a patient comprising administering to said patient compound 9 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 9 or a composition thereof. In some embodiments, the present disclosure provides a method of treating a disease, disorder or condition in a patient, comprising administering to said patient compound 9 or a composition thereof, wherein the disease, disorder or condition is mediated by IRAK4. In some embodiments, the present disclosure provides a method of treating an autoimmune or inflammatory disorder in a patient, comprising administering to said patient compound 9 or a composition thereof.

### Compound 10 (Citric Acid Salts of Compound 1)

According to one embodiment, the present disclosure provides a citric acid salt of compound 1, represented by compound 10:

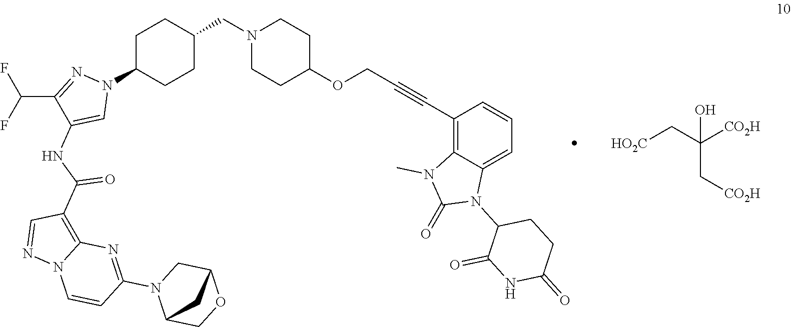 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CC(O)(CC(=O)O)C(=O)O]

It will be appreciated by one of ordinary skill in the art that the citric acid and compound 1 are ionically bonded to form compound 10. In some embodiments, compound 10 is a dihydrogen citrate salt. In some embodiments, compound 10 is a hydrogen citrate salt. In some embodiments, compound 10 is a citrate salt. In some embodiments, compound 10 exists as a mixture of dihydrogen citrate, hydrogen citrate, and citrate salts. It is contemplated that compound 10 can exist in a variety of physical forms. For example, compound 10 can be in solution, suspension, or in solid form. In certain embodiments, compound 10 is in solid form. When compound 10 is in solid form, said compound may be amorphous, crystalline, or a mixture thereof. Exemplary solid forms are described in more detail below.

In some embodiments, the present disclosure provides compound 10 substantially free of impurities. Such impurities or extraneous matter may include different forms of compound 10, residual solvents, or any other impurities that may result from the preparation of, and/or isolation of, compound 10. In certain embodiments, at least about 95% by weight of a form of compound 10 is present. In still other embodiments of the disclosure, at least about 99% by weight of a form of compound 10 is present.

According to one embodiment, a form of compound 10 is present in an amount of at least about 97, 97.5, 98.0, 98.5, 99, 99.5, 99.8 weight percent where the percentages are based on the total weight of the composition. According to another embodiment, a form of compound 10 contains no more than about 3.0 area percent HPLC of total organic impurities and, in certain embodiments, no more than about 1.5 area percent HPLC total organic impurities relative to the total area of the HPLC chromatogram. In other embodiments, a form of compound 10 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 10 is also meant to include all tautomeric forms of compound 10. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 10 can exist in a variety of solid forms. Exemplary such forms include polymorphs such as those described herein.

In certain embodiments, compound 10 is a crystalline solid. In other embodiments, compound 10 is a crystalline solid substantially free of amorphous compound 10. As used herein, the term “substantially free of amorphous compound 10” means that the compound contains no significant amount of amorphous compound 10. In certain embodiments, at least about 95% by weight of crystalline compound 10 is present. In still other embodiments of the disclosure, at least about 99% by weight of crystalline compound 10 is present.

It has been found that compound 10 can exist in at least one distinct polymorphic form. In certain embodiments, the present disclosure provides a polymorphic form of compound 10 referred to herein as Form A.

### Form A of Compound 10

Methods for preparing Form A of compound 10 are described infra.

In some embodiments, the disclosure provides compound 10:

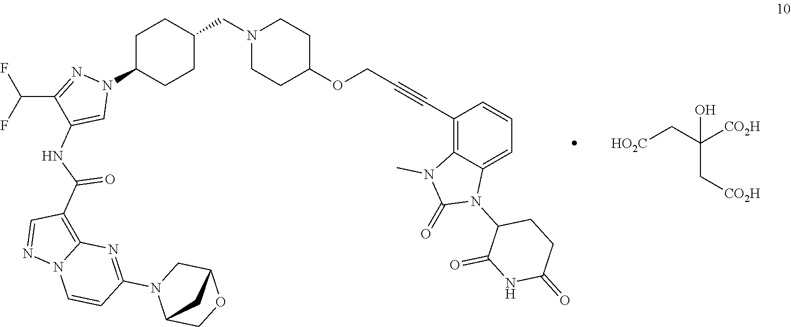 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CC(O)(CC(=O)O)C(=O)O]

wherein said compound is crystalline. In some embodiments, the present disclosure provides compound 10, wherein said compound is substantially free of amorphous compound 10. In some embodiments, the present disclosure provides compound 10, wherein said compound is substantially free of amorphous compound 1. In some embodiments, the present disclosure provides compound 10, wherein said compound is substantially free of a crystalline form of compound 1. In some embodiments, the present disclosure provides compound 10, wherein said compound is substantially free of impurities.

In some embodiments, Form A of compound 10 is characterized by one or more peaks in its XRPD pattern selected from those at 6.0, 11.9, and 16.5±0.5 degree 2-theta. In some embodiments, Form A of compound 10 is characterized by one or more peaks in its XRPD pattern selected from those at 6.0, 11.9, and 16.5±0.4 degree 2-theta. In some embodiments, Form A of compound 10 is characterized by one or more peaks in its XRPD pattern selected from those at 6.0, 11.9, and 16.5±0.3 degree 2-theta. In some embodiments, Form A of compound 10 is characterized by one or more peaks in its XRPD pattern selected from those at 6.0, 11.9, and 16.5±0.2 degree 2-theta.

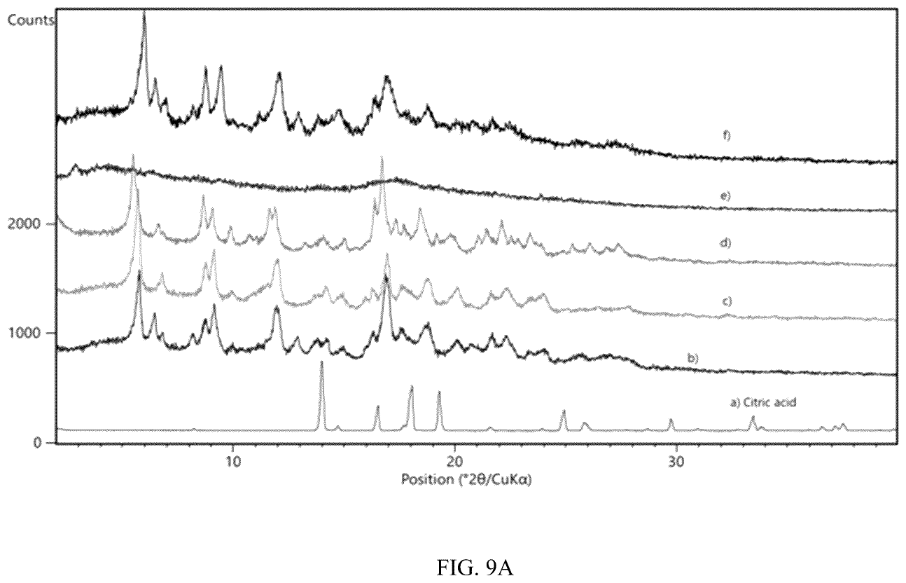

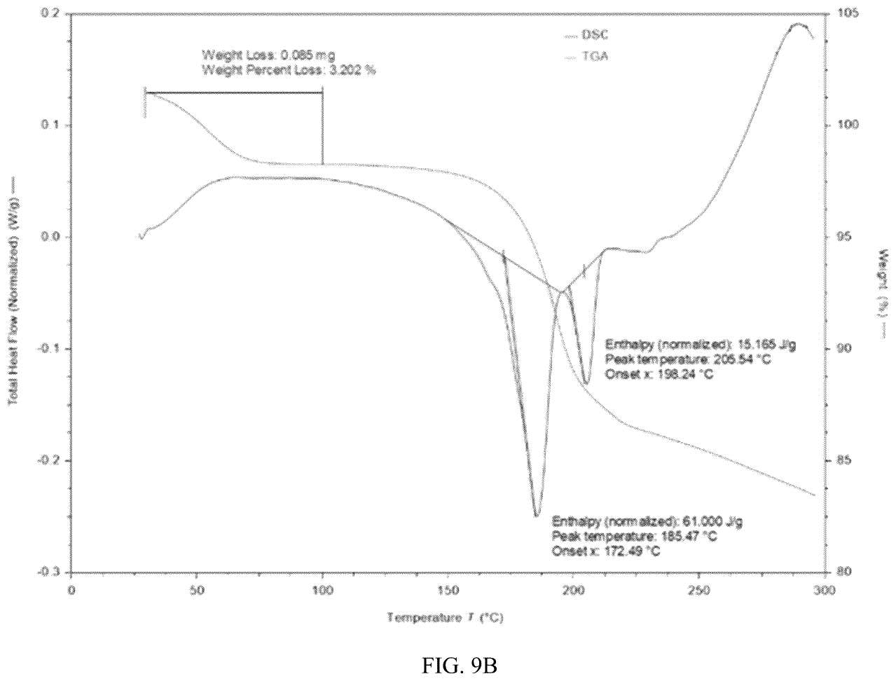

In some embodiments, the present disclosure provides compound 10, wherein said compound has an XRPD substantially similar (e.g., having peaks ±0.2 degree 2-theta) to that depicted in FIG. 9A.

In some embodiments, the present disclosure provides compound 10, wherein said compound has a DSC-TGA substantially similar (e.g., having an endothermic DSC event onset at 171-176° C.) to that depicted in FIG. 9B. In some embodiments, the present disclosure provides compound 10, wherein said compound has a DSC event and/or a TGA weight loss as described in Table 6.

In some embodiments, the present disclosure provides a composition comprising compound 10 and a pharmaceutically acceptable carrier or excipient.

In some embodiments, the present disclosure provides a method of modulating IRAK4 kinase in a patient comprising administering to said patient compound 10 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 10 or a composition thereof. In some embodiments, the present disclosure provides a method of treating a disease, disorder or condition in a patient, comprising administering to said patient compound 10 or a composition thereof, wherein the disease, disorder or condition is mediated by IRAK4. In some embodiments, the present disclosure provides a method of treating an autoimmune or inflammatory disorder in a patient, comprising administering to said patient compound 10 or a composition thereof.

### Compound 11 (Glycolic Acid Salts of Compound 1)

According to one embodiment, the present disclosure provides a glycolic acid salt of compound 1, represented by compound 11:

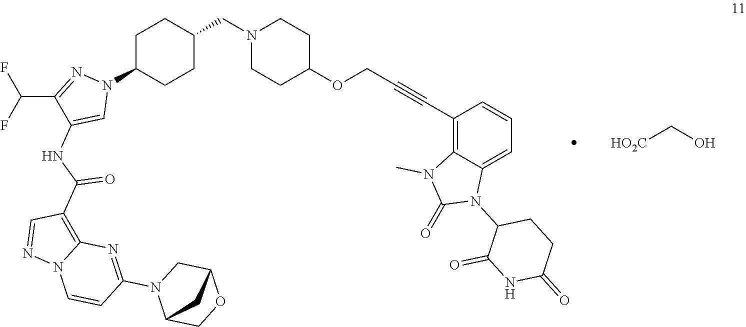 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CO]

It will be appreciated by one of ordinary skill in the art that the glycolic acid and compound 1 are ionically bonded to form compound 11. In some embodiments, compound 11 is a glycolate salt. It is contemplated that compound 11 can exist in a variety of physical forms. For example, compound 11 can be in solution, suspension, or in solid form. In certain embodiments, compound 11 is in solid form. When compound 11 is in solid form, said compound may be amorphous, crystalline, or a mixture thereof. Exemplary solid forms are described in more detail below.

In some embodiments, the present disclosure provides compound 11 substantially free of impurities. Such impurities or extraneous matter may include different forms of compound 11, residual solvents, or any other impurities that may result from the preparation of, and/or isolation of, compound 11. In certain embodiments, at least about 95% by weight of a form of compound 11 is present. In still other embodiments of the disclosure, at least about 99% by weight of a form of compound 11 is present.

According to one embodiment, a form of compound 11 is present in an amount of at least about 97, 97.5, 98.0, 98.5, 99, 99.5, 99.8 weight percent where the percentages are based on the total weight of the composition. According to another embodiment, a form of compound 11 contains no more than about 3.0 area percent HPLC of total organic impurities and, in certain embodiments, no more than about 1.5 area percent HPLC total organic impurities relative to the total area of the HPLC chromatogram. In other embodiments, a form of compound 11 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 11 is also meant to include all tautomeric forms of compound 11. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 11 can exist in a variety of solid forms. Exemplary such forms include polymorphs such as those described herein.

In certain embodiments, compound 11 is a crystalline solid. In other embodiments, compound 11 is a crystalline solid substantially free of amorphous compound 11. As used herein, the term “substantially free of amorphous compound 11” means that the compound contains no significant amount of amorphous compound 11. In certain embodiments, at least about 95% by weight of crystalline compound 11 is present. In still other embodiments of the disclosure, at least about 99% by weight of crystalline compound 11 is present.

It has been found that compound 11 can exist in at least one distinct polymorphic form. In certain embodiments, the present disclosure provides a polymorphic form of compound 11 referred to herein as Form A.

### Form A of Compound 11

Methods for preparing Form A of compound 11 are described infra.

In some embodiments, the disclosure provides compound 11:

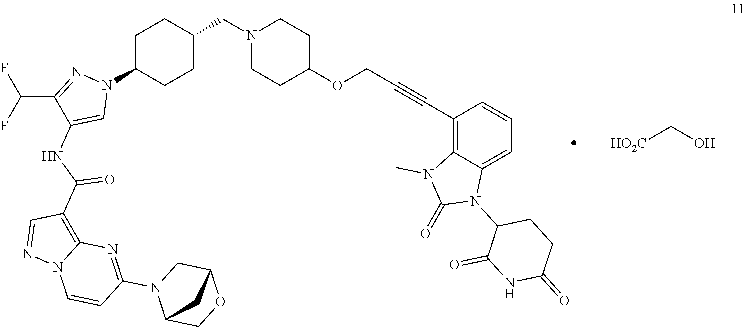 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CO]

wherein said compound is crystalline. In some embodiments, the present disclosure provides compound 11, wherein said compound is substantially free of amorphous compound 11. In some embodiments, the present disclosure provides compound 11, wherein said compound is substantially free of amorphous compound 1. In some embodiments, the present disclosure provides compound 11, wherein said compound is substantially free of a crystalline form of compound 1. In some embodiments, the present disclosure provides compound 11, wherein said compound is substantially free of impurities.

In some embodiments, Form A of compound 11 is characterized by one or more peaks in its XRPD pattern selected from those at 6.8, 13.0, and 17.1±0.5 degree 2-theta. In some embodiments, Form A of compound 11 is characterized by one or more peaks in its XRPD pattern selected from those at 6.8, 13.0, and 17.1±0.4 degree 2-theta. In some embodiments, Form A of compound 11 is characterized by one or more peaks in its XRPD pattern selected from those at 6.8, 13.0, and 17.1±0.3 degree 2-theta. In some embodiments, Form A of compound 11 is characterized by one or more peaks in its XRPD pattern selected from those at 6.8, 13.0, and 17.1±0.2 degree 2-theta.

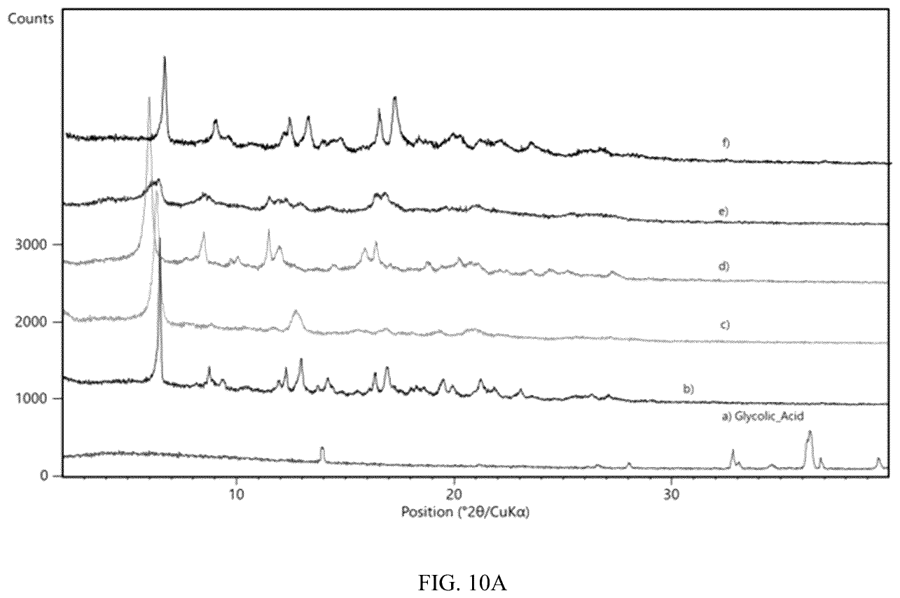

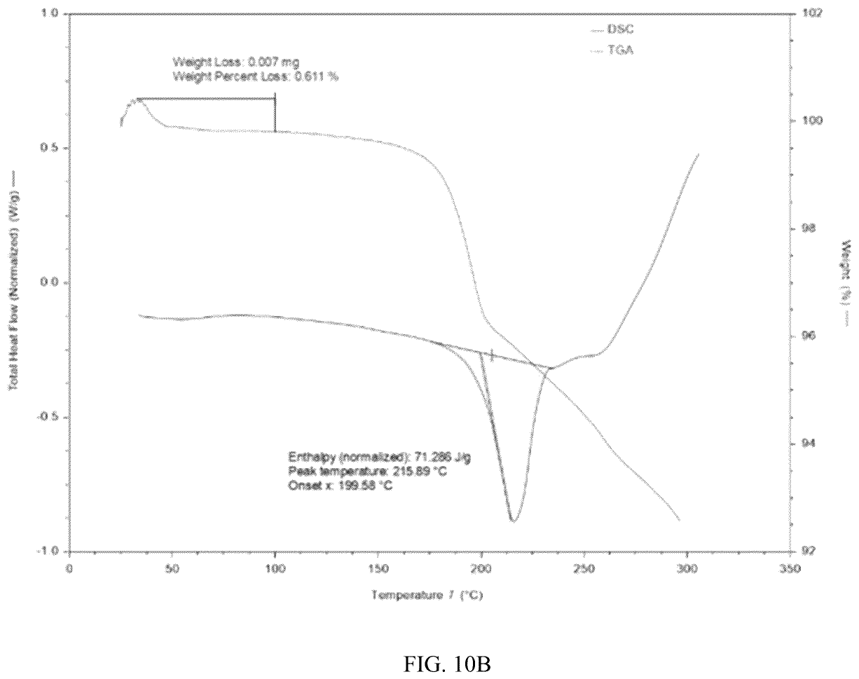

In some embodiments, the present disclosure provides compound 11, wherein said compound has an XRPD substantially similar (e.g., having peaks ±0.2 degree 2-theta) to that depicted in FIG. 10A.

In some embodiments, the present disclosure provides compound 11, wherein said compound has a DSC-TGA substantially similar (e.g., having an endothermic DSC event onset at 197-202° C.) to that depicted in FIG. 10B. In some embodiments, the present disclosure provides compound 11, wherein said compound has a DSC event and/or a TGA weight loss as described in Table 6.

In some embodiments, the present disclosure provides a composition comprising compound 12 and a pharmaceutically acceptable carrier or excipient.

In some embodiments, the present disclosure provides a method of modulating IRAK4 kinase in a patient comprising administering to said patient compound 11 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 11 or a composition thereof. In some embodiments, the present disclosure provides a method of treating a disease, disorder or condition in a patient, comprising administering to said patient compound 11 or a composition thereof, wherein the disease, disorder or condition is mediated by IRAK4. In some embodiments, the present disclosure provides a method of treating an autoimmune or inflammatory disorder in a patient, comprising administering to said patient compound 11 or a composition thereof.

### Compound 12 (L-Malic Acid Salts of Compound 1)

According to one embodiment, the present disclosure provides a L-malic acid salt of compound 1, represented by compound 12:

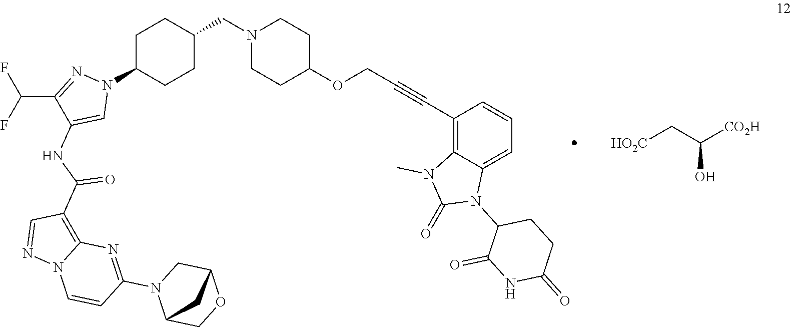 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)C[C@H](O)C(=O)O]

It will be appreciated by one of ordinary skill in the art that the L-malic acid and compound 1 are ionically bonded to form compound 12. In some embodiments, compound 12 is a hydrogen L-malate salt. In some embodiments, compound 12 is a L-malate salt. It is contemplated that compound 12 can exist in a variety of physical forms. For example, compound 12 can be in solution, suspension, or in solid form. In certain embodiments, compound 12 is in solid form. When compound 12 is in solid form, said compound may be amorphous, crystalline, or a mixture thereof. Exemplary solid forms are described in more detail below.

In some embodiments, the present disclosure provides compound 12 substantially free of impurities. Such impurities or extraneous matter may include different forms of compound 12, residual solvents, or any other impurities that may result from the preparation of, and/or isolation of, compound 12. In certain embodiments, at least about 95% by weight of a form of compound 12 is present. In still other embodiments of the disclosure, at least about 99% by weight of a form of compound 12 is present.

According to one embodiment, a form of compound 12 is present in an amount of at least about 97, 97.5, 98.0, 98.5, 99, 99.5, 99.8 weight percent where the percentages are based on the total weight of the composition. According to another embodiment, a form of compound 12 contains no more than about 3.0 area percent HPLC of total organic impurities and, in certain embodiments, no more than about 1.5 area percent HPLC total organic impurities relative to the total area of the HPLC chromatogram. In other embodiments, a form of compound 12 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 12 is also meant to include all tautomeric forms of compound 12. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 12 can exist in a variety of solid forms. Exemplary such forms include polymorphs such as those described herein.

In certain embodiments, compound 12 is a crystalline solid. In other embodiments, compound 12 is a crystalline solid substantially free of amorphous compound 12. As used herein, the term “substantially free of amorphous compound 12” means that the compound contains no significant amount of amorphous compound 12. In certain embodiments, at least about 95% by weight of crystalline compound 12 is present. In still other embodiments of the disclosure, at least about 99% by weight of crystalline compound 12 is present.

It has been found that compound 12 can exist in at least one distinct polymorphic form. In certain embodiments, the present disclosure provides a polymorphic form of compound 12 referred to herein as Form A.

### Form A of Compound 12

Methods for preparing Form A of compound 12 are described infra.

In some embodiments, the disclosure provides compound 12:

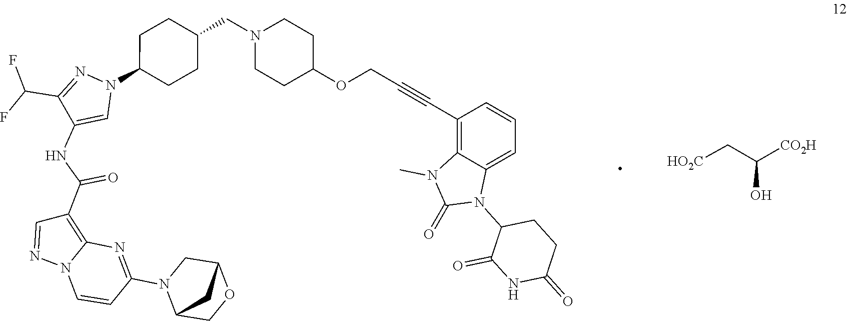 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)C[C@H](O)C(=O)O]

wherein said compound is crystalline. In some embodiments, the present disclosure provides compound 12, wherein said compound is substantially free of amorphous compound 12. In some embodiments, the present disclosure provides compound 12, wherein said compound is substantially free of amorphous compound 1. In some embodiments, the present disclosure provides compound 12, wherein said compound is substantially free of a crystalline form of compound 1. In some embodiments, the present disclosure provides compound 12, wherein said compound is substantially free of impurities.

In some embodiments, Form A of compound 12 is characterized by one or more peaks in its XRPD pattern selected from those at 6.5, 10.8, and 16.8±0.5 degree 2-theta. In some embodiments, Form A of compound 12 is characterized by one or more peaks in its XRPD pattern selected from those at 6.5, 10.8, and 16.8±0.4 degree 2-theta. In some embodiments, Form A of compound 12 is characterized by one or more peaks in its XRPD pattern selected from those at 6.5, 10.8, and 16.8±0.3 degree 2-theta. In some embodiments, Form A of compound 12 is characterized by one or more peaks in its XRPD pattern selected from those at 6.5, 10.8, and 16.8±0.2 degree 2-theta.

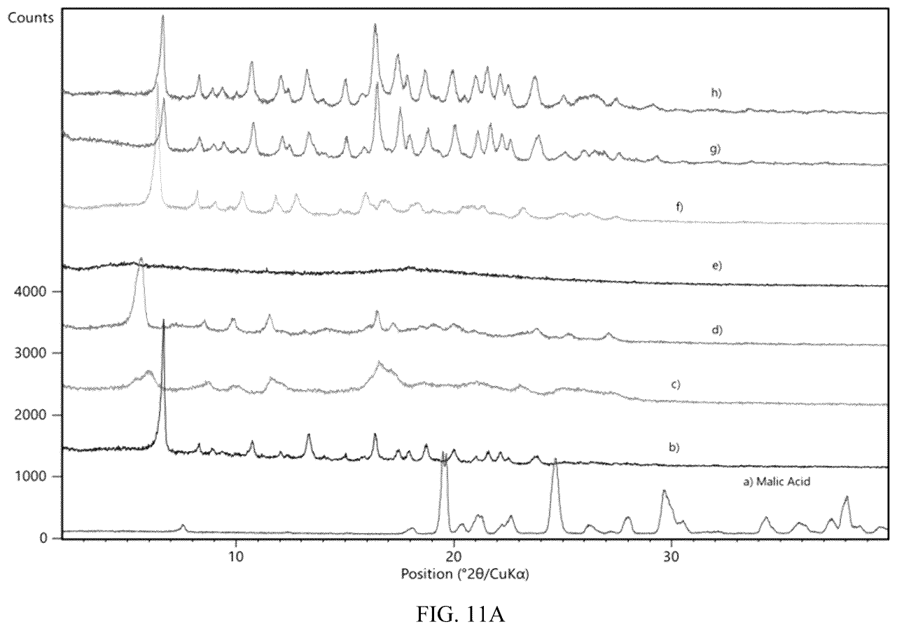

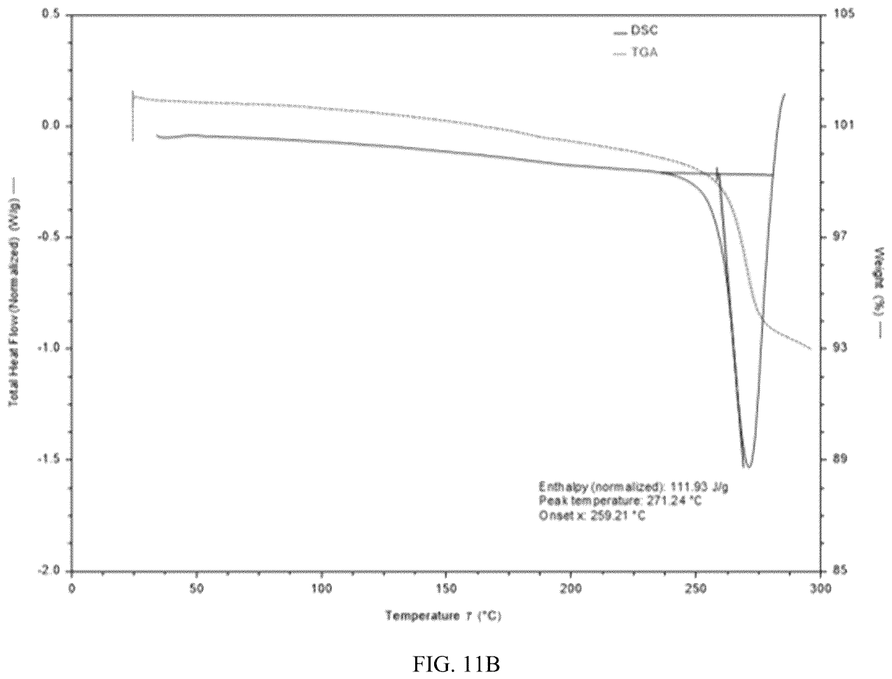

In some embodiments, the present disclosure provides compound 12, wherein said compound has an XRPD substantially similar (e.g., having peaks ±0.2 degree 2-theta) to that depicted in FIG. 11A.

In some embodiments, the present disclosure provides compound 12, wherein said compound has a DSC-TGA substantially similar (e.g., having an endothermic DSC event onset at 257-262° C.) to that depicted in FIG. 11B. In some embodiments, the present disclosure provides compound 12, wherein said compound has a DSC event and/or a TGA weight loss as described in Table 6.

In some embodiments, the present disclosure provides a composition comprising compound 12 and a pharmaceutically acceptable carrier or excipient.

In some embodiments, the present disclosure provides a method of modulating IRAK4 kinase in a patient comprising administering to said patient compound 12 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 12 or a composition thereof. In some embodiments, the present disclosure provides a method of treating a disease, disorder or condition in a patient, comprising administering to said patient compound 12 or a composition thereof, wherein the disease, disorder or condition is mediated by IRAK4. In some embodiments, the present disclosure provides a method of treating an autoimmune or inflammatory disorder in a patient, comprising administering to said patient compound 12 or a composition thereof.

### Compound 13 (Hippuric Acid Salts of Compound 1)

According to one embodiment, the present disclosure provides a hippuric acid salt of compound 1, represented by compound 13:

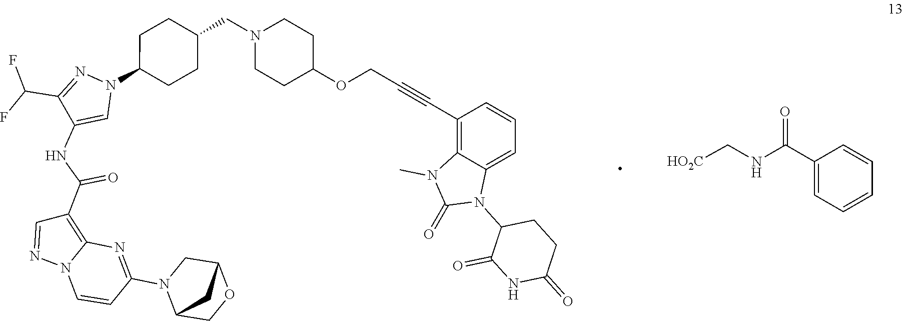 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CNC(=O)c1ccccc1]

It will be appreciated by one of ordinary skill in the art that the hippuric acid and compound 1 are ionically bonded to form compound 13. In some embodiments, compound 13 is a hippurate salt. It is contemplated that compound 13 can exist in a variety of physical forms. For example, compound 13 can be in solution, suspension, or in solid form. In certain embodiments, compound 13 is in solid form. When compound 13 is in solid form, said compound may be amorphous, crystalline, or a mixture thereof. Exemplary solid forms are described in more detail below.

In some embodiments, the present disclosure provides compound 13 substantially free of impurities. Such impurities or extraneous matter may include different forms of compound 13, residual solvents, or any other impurities that may result from the preparation of, and/or isolation of, compound 13. In certain embodiments, at least about 95% by weight of a form of compound 13 is present. In still other embodiments of the disclosure, at least about 99% by weight of a form of compound 15 is present.

According to one embodiment, a form of compound 13 is present in an amount of at least about 97, 97.5, 98.0, 98.5, 99, 99.5, 99.8 weight percent where the percentages are based on the total weight of the composition. According to another embodiment, a form of compound 13 contains no more than about 3.0 area percent HPLC of total organic impurities and, in certain embodiments, no more than about 1.5 area percent HPLC total organic impurities relative to the total area of the HPLC chromatogram. In other embodiments, a form of compound 13 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 13 is also meant to include all tautomeric forms of compound 13. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 13 can exist in a variety of solid forms. Exemplary such forms include polymorphs such as those described herein.

In certain embodiments, compound 13 is a crystalline solid. In other embodiments, compound 13 is a crystalline solid substantially free of amorphous compound 13. As used herein, the term “substantially free of amorphous compound 13” means that the compound contains no significant amount of amorphous compound 13. In certain embodiments, at least about 95% by weight of crystalline compound 13 is present. In still other embodiments of the disclosure, at least about 99% by weight of crystalline compound 13 is present.

It has been found that compound 13 can exist in at least one distinct polymorphic form. In certain embodiments, the present disclosure provides a polymorphic form of compound 13 referred to herein as Form A.

### Form A of Compound 13

Methods for preparing Form A of compound 13 are described infra.

In some embodiments, the disclosure provides compound 13:

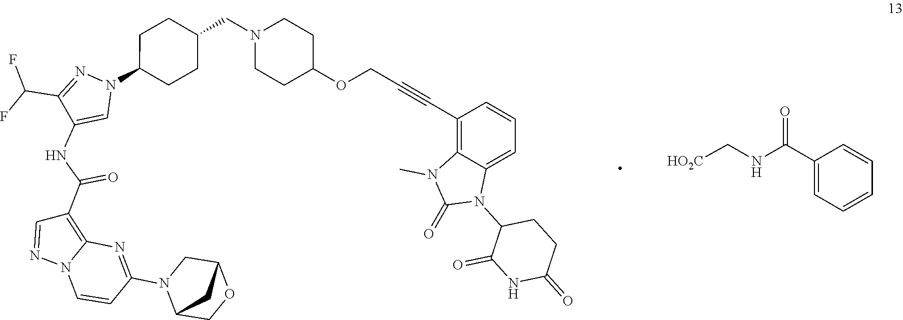 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CNC(=O)c1ccccc1]

wherein said compound is crystalline. In some embodiments, the present disclosure provides compound 13, wherein said compound is substantially free of amorphous compound 13. In some embodiments, the present disclosure provides compound 13, wherein said compound is substantially free of amorphous compound 1. In some embodiments, the present disclosure provides compound 13, wherein said compound is substantially free of a crystalline form of compound 1. In some embodiments, the present disclosure provides compound 13, wherein said compound is substantially free of impurities.

In some embodiments, Form A of compound 13 is characterized by one or more peaks in its XRPD pattern selected from those at 6.0, 16.3, and 20.1±0.5 degree 2-theta. In some embodiments, Form A of compound 13 is characterized by one or more peaks in its XRPD pattern selected from those at 6.0, 16.3, and 20.1±0.4 degree 2-theta. In some embodiments, Form A of compound 13 is characterized by one or more peaks in its XRPD pattern selected from those at 6.0, 16.3, and 20.1±0.3 degree 2-theta. In some embodiments, Form A of compound 13 is characterized by one or more peaks in its XRPD pattern selected from those at 6.0, 16.3, and 20.1±0.2 degree 2-theta.

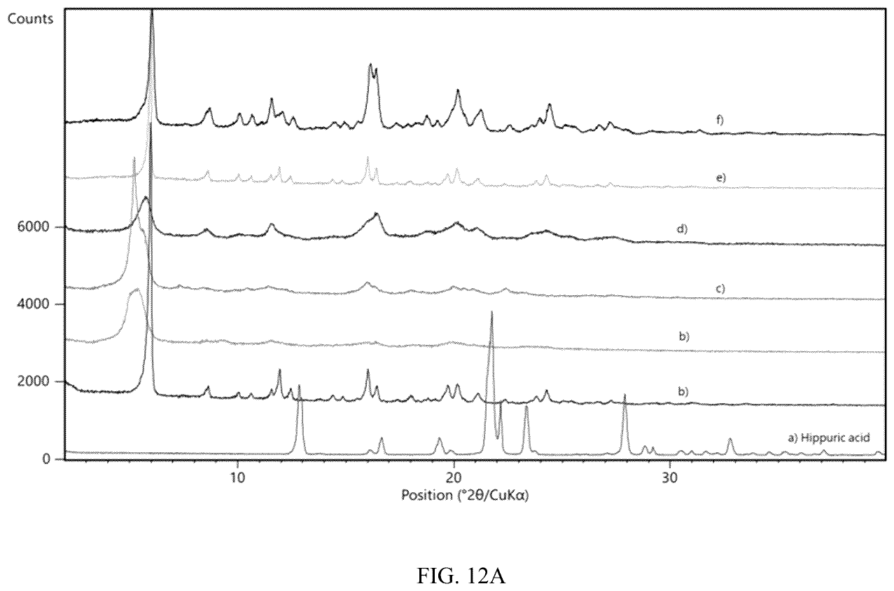

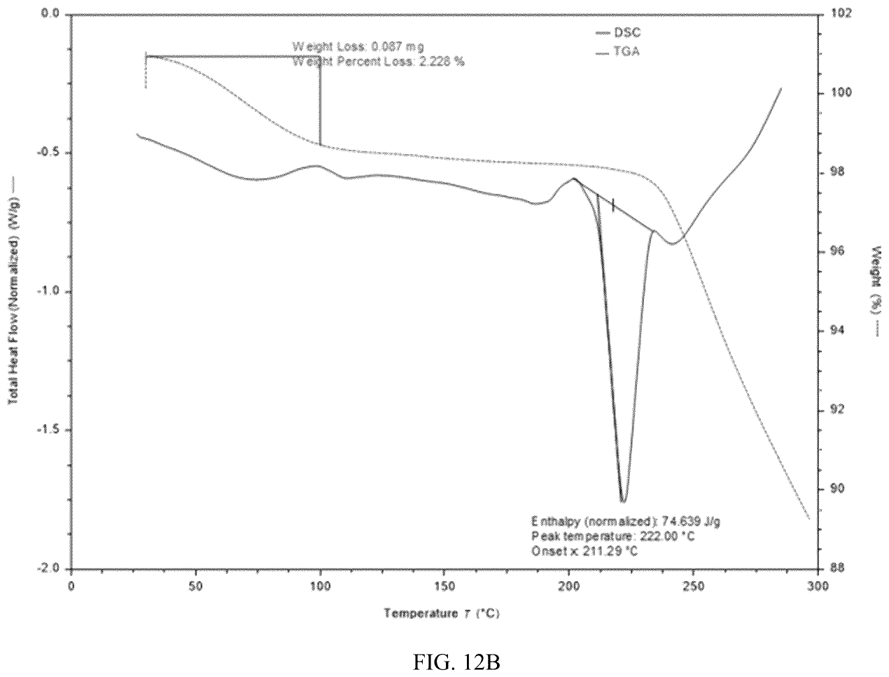

In some embodiments, the present disclosure provides compound 13, wherein said compound has an XRPD substantially similar (e.g., having peaks ±0.2 degree 2-theta) to that depicted in FIG. 12A.

In some embodiments, the present disclosure provides compound 13, wherein said compound has a DSC-TGA substantially similar (e.g., having an endothermic DSC event onset at 199-204° C.) to that depicted in FIG. 12B. In some embodiments, the present disclosure provides compound 13, wherein said compound has a DSC event and/or a TGA weight loss as described in Table 6.

In some embodiments, the present disclosure provides a composition comprising compound 13 and a pharmaceutically acceptable carrier or excipient.

In some embodiments, the present disclosure provides a method of modulating IRAK4 kinase in a patient comprising administering to said patient compound 13 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 13 or a composition thereof. In some embodiments, the present disclosure provides a method of treating a disease, disorder or condition in a patient, comprising administering to said patient compound 13 or a composition thereof, wherein the disease, disorder or condition is mediated by IRAK4. In some embodiments, the present disclosure provides a method of treating an autoimmune or inflammatory disorder in a patient, comprising administering to said patient compound 13 or a composition thereof.

### Compound 14 (L-Lactic Acid Salts of Compound 1)

According to one embodiment, the present disclosure provides a L-lactic acid salt of compound 1, represented by compound 14:

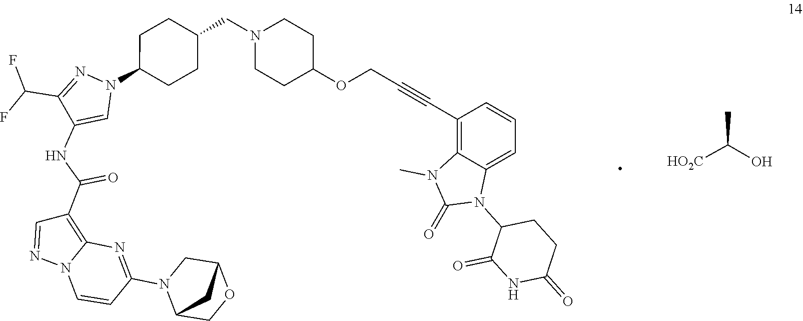

It will be appreciated by one of ordinary skill in the art that the L-lactic acid and compound 1 are ionically bonded to form compound 14. In some embodiments, compound 14 is a lactate salt. It is contemplated that compound 14 can exist in a variety of physical forms. For example, compound 14 can be in solution, suspension, or in solid form. In certain embodiments, compound 14 is in solid form. When compound 14 is in solid form, said compound may be amorphous, crystalline, or a mixture thereof. Exemplary solid forms are described in more detail below.

In some embodiments, the present disclosure provides compound 14 substantially free of impurities. Such impurities or extraneous matter may include different forms of compound 14, residual solvents, or any other impurities that may result from the preparation of, and/or isolation of, compound 14. In certain embodiments, at least about 95% by weight of a form of compound 14 is present. In still other embodiments of the disclosure, at least about 99% by weight of a form of compound 14 is present.

According to one embodiment, a form of compound 14 is present in an amount of at least about 97, 97.5, 98.0, 98.5, 99, 99.5, 99.8 weight percent where the percentages are based on the total weight of the composition. According to another embodiment, a form of compound 14 contains no more than about 3.0 area percent HPLC of total organic impurities and, in certain embodiments, no more than about 1.5 area percent HPLC total organic impurities relative to the total area of the HPLC chromatogram. In other embodiments, a form of compound 14 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 14 is also meant to include all tautomeric forms of compound 14. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 14 can exist in a variety of solid forms. Exemplary such forms include polymorphs such as those described herein.

In certain embodiments, compound 16 is a crystalline solid. In other embodiments, compound 14 is a crystalline solid substantially free of amorphous compound 14. As used herein, the term “substantially free of amorphous compound 14” means that the compound contains no significant amount of amorphous compound 14. In certain embodiments, at least about 95% by weight of crystalline compound 14 is present. In still other embodiments of the disclosure, at least about 99% by weight of crystalline compound 16 is present.

It has been found that compound 14 can exist in at least one distinct polymorphic form. In certain embodiments, the present disclosure provides a polymorphic form of compound 14 referred to herein as Form A.

### Form A of Compound 14

Methods for preparing Form A of compound 14 are described infra.

In some embodiments, the disclosure provides compound 14:

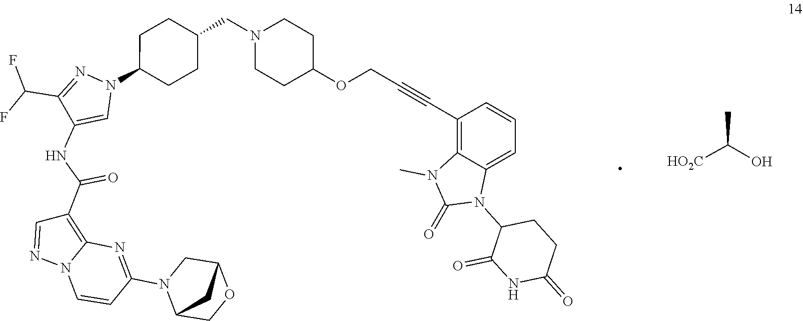

wherein said compound is crystalline. In some embodiments, the present disclosure provides compound 14, wherein said compound is substantially free of amorphous compound 14. In some embodiments, the present disclosure provides compound 14, wherein said compound is substantially free of amorphous compound 1. In some embodiments, the present disclosure provides compound 14, wherein said compound is substantially free of a crystalline form of compound 1. In some embodiments, the present disclosure provides compound 14, wherein said compound is substantially free of impurities.

In some embodiments, Form A of compound 14 is characterized by one or more peaks in its XRPD pattern selected from those at 6.5, 17.1, and 21.0±0.5 degree 2-theta. In some embodiments, Form A of compound 14 is characterized by one or more peaks in its XRPD pattern selected from those at 6.5, 17.1, and 21.0±0.4 degree 2-theta. In some embodiments, Form A of compound 14 is characterized by one or more peaks in its XRPD pattern selected from those at 6.5, 17.1, and 21.0±0.3 degree 2-theta. In some embodiments, Form A of compound 14 is characterized by one or more peaks in its XRPD pattern selected from those at 6.5, 17.1, and 21.0±0.2 degree 2-theta.

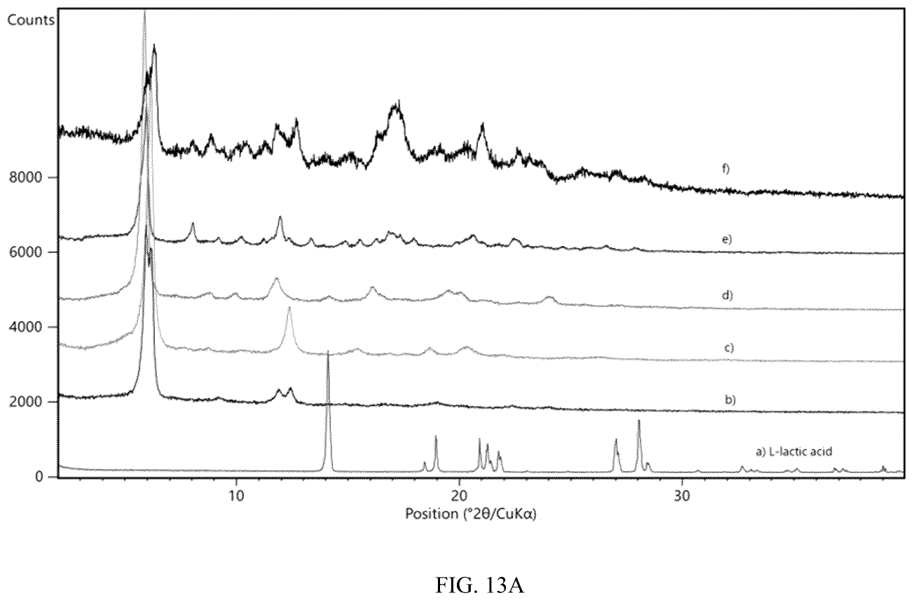

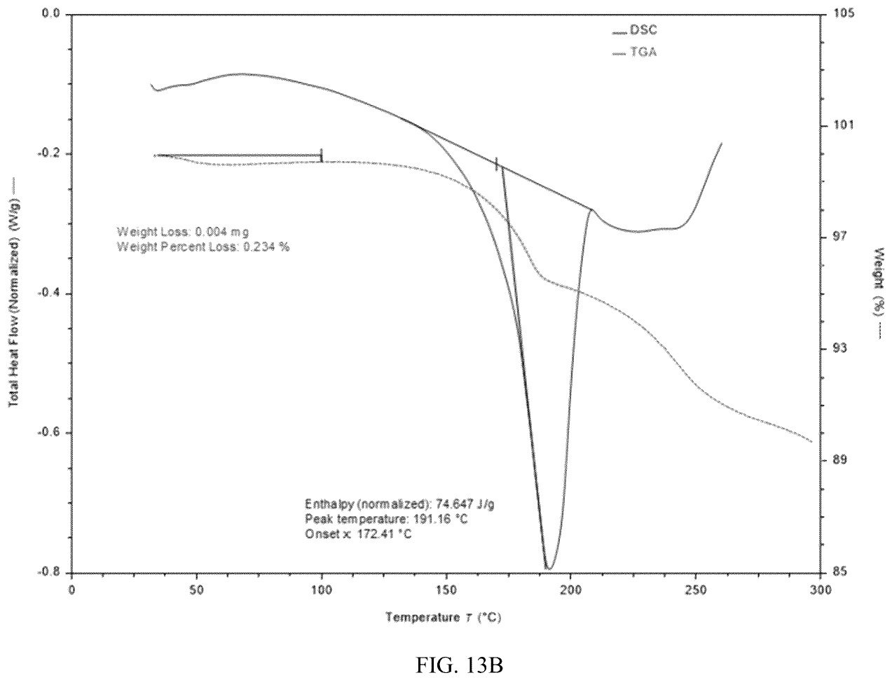

In some embodiments, the present disclosure provides compound 14, wherein said compound has an XRPD substantially similar (e.g., having peaks ±0.2 degree 2-theta) to that depicted in FIG. 13A.

In some embodiments, the present disclosure provides compound 14, wherein said compound has a DSC-TGA substantially similar (e.g., having an endothermic DSC event onset at 170-175° C.) to that depicted in FIG. 13B. In some embodiments, the present disclosure provides compound 14, wherein said compound has a DSC event and/or a TGA weight loss as described in Table 6.

In some embodiments, the present disclosure provides a composition comprising compound 14 and a pharmaceutically acceptable carrier or excipient.

In some embodiments, the present disclosure provides a method of modulating IRAK4 kinase in a patient comprising administering to said patient compound 14 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 14 or a composition thereof. In some embodiments, the present disclosure provides a method of treating a disease, disorder or condition in a patient, comprising administering to said patient compound 14 or a composition thereof, wherein the disease, disorder or condition is mediated by IRAK4. In some embodiments, the present disclosure provides a method of treating an autoimmune or inflammatory disorder in a patient, comprising administering to said patient compound 14 or a composition thereof.

### Compound 15 (Benzoic Acid Salts of Compound 1)

According to one embodiment, the present disclosure provides a benzoic acid salt of compound 1, represented by compound 15:

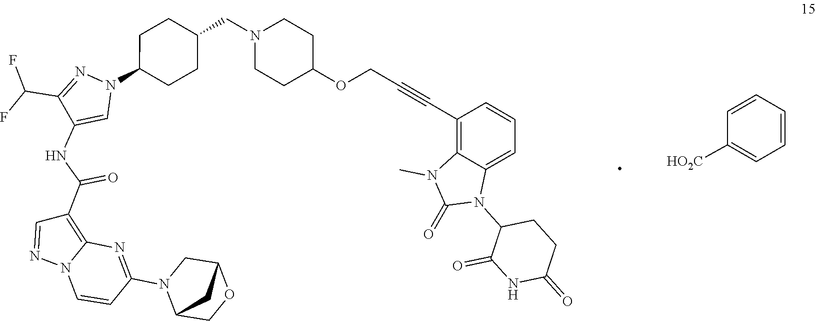 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)c1ccccc1]

It will be appreciated by one of ordinary skill in the art that the benzoic acid and compound 1 are ionically bonded to form compound 15. In some embodiments, compound 15 is a benzoate salt. It is contemplated that compound 15 can exist in a variety of physical forms. For example, compound 15 can be in solution, suspension, or in solid form. In certain embodiments, compound 15 is in solid form. When compound 15 is in solid form, said compound may be amorphous, crystalline, or a mixture thereof. Exemplary solid forms are described in more detail below.

In some embodiments, the present disclosure provides compound 15 substantially free of impurities. Such impurities or extraneous matter may include different forms of compound 15, residual solvents, or any other impurities that may result from the preparation of, and/or isolation of, compound 15. In certain embodiments, at least about 95% by weight of a form of compound 15 is present. In still other embodiments of the disclosure, at least about 99% by weight of a form of compound 15 is present.

According to one embodiment, a form of compound 15 is present in an amount of at least about 97, 97.5, 98.0, 98.5, 99, 99.5, 99.8 weight percent where the percentages are based on the total weight of the composition. According to another embodiment, a form of compound 15 contains no more than about 3.0 area percent HPLC of total organic impurities and, in certain embodiments, no more than about 1.5 area percent HPLC total organic impurities relative to the total area of the HPLC chromatogram. In other embodiments, a form of compound 15 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 15 is also meant to include all tautomeric forms of compound 15. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 15 can exist in a variety of solid forms. Exemplary such forms include polymorphs such as those described herein.

In certain embodiments, compound 15 is a crystalline solid. In other embodiments, compound 15 is a crystalline solid substantially free of amorphous compound 15. As used herein, the term “substantially free of amorphous compound 15” means that the compound contains no significant amount of amorphous compound 15. In certain embodiments, at least about 95% by weight of crystalline compound 15 is present. In still other embodiments of the disclosure, at least about 99% by weight of crystalline compound 15 is present.

It has been found that compound 15 can exist in at least one distinct polymorphic form. In certain embodiments, the present disclosure provides a polymorphic form of compound 15 referred to herein as Form A.

### Form A of Compound 15

Methods for preparing Form A of compound 15 are described infra.

In some embodiments, the disclosure provides compound 15:

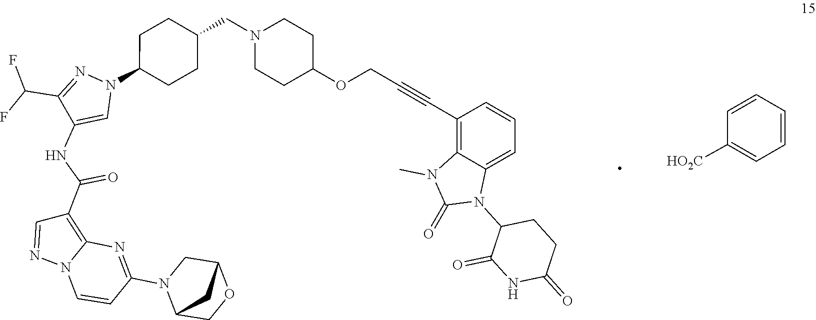 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)c1ccccc1]

wherein said compound is crystalline. In some embodiments, the present disclosure provides compound 15, wherein said compound is substantially free of amorphous compound 15. In some embodiments, the present disclosure provides compound 15, wherein said compound is substantially free of amorphous compound 1. In some embodiments, the present disclosure provides compound 15, wherein said compound is substantially free of a crystalline form of compound 1. In some embodiments, the present disclosure provides compound 15, wherein said compound is substantially free of impurities.

In some embodiments, Form A of compound 15 is characterized by one or more peaks in its XRPD pattern selected from those at 6.0, 17.0, and 20.2±0.5 degree 2-theta. In some embodiments, Form A of compound 15 is characterized by one or more peaks in its XRPD pattern selected from those at 6.0, 17.0, and 20.2±0.4 degree 2-theta. In some embodiments, Form A of compound 15 is characterized by one or more peaks in its XRPD pattern selected from those at 6.0, 17.0, and 20.2±0.3 degree 2-theta. In some embodiments, Form A of compound 15 is characterized by one or more peaks in its XRPD pattern selected from those at 6.0, 17.0, and 20.2±0.2 degree 2-theta.

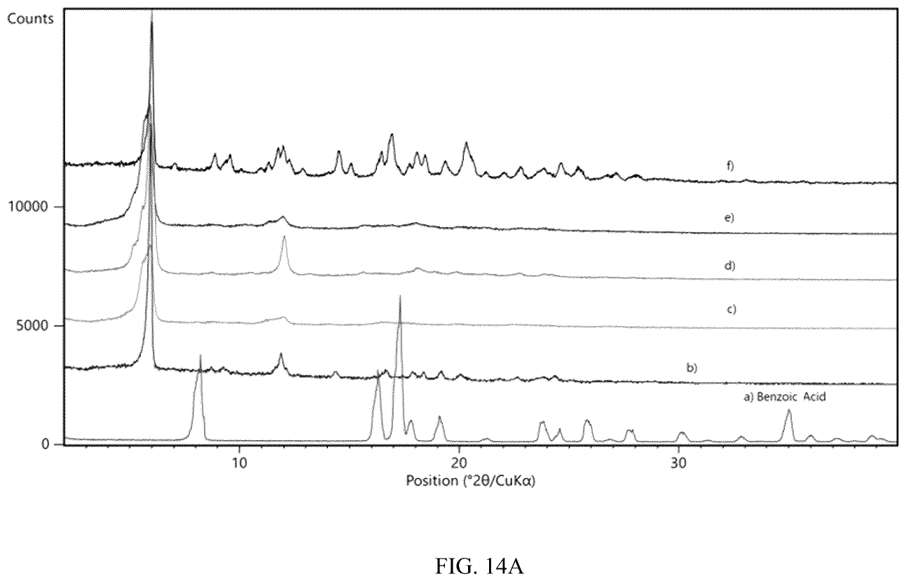

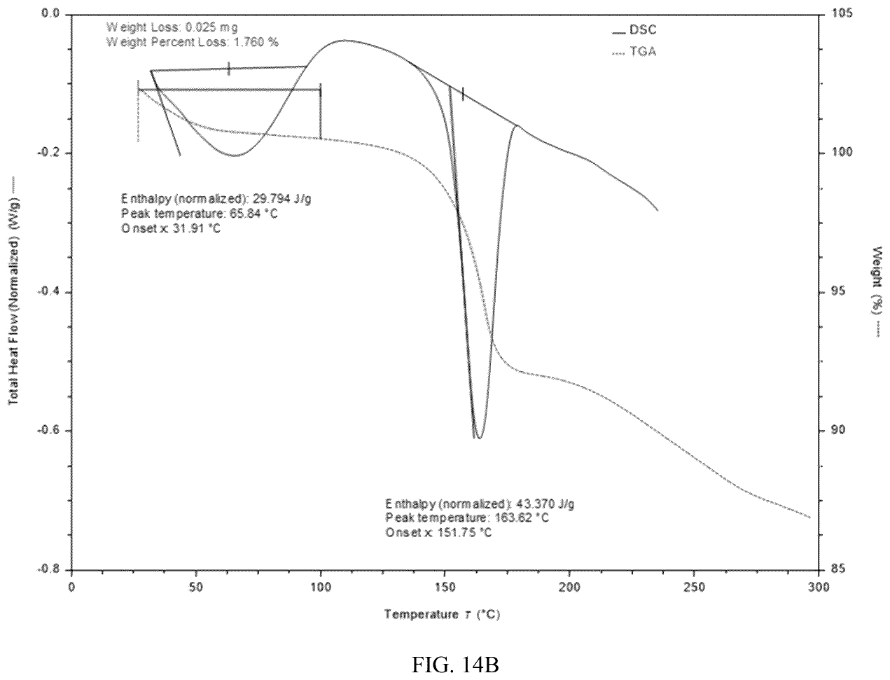

In some embodiments, the present disclosure provides compound 15, wherein said compound has an XRPD substantially similar (e.g., having peaks ±0.2 degree 2-theta) to that depicted in FIG. 14A.

In some embodiments, the present disclosure provides compound 15, wherein said compound has a DSC-TGA substantially similar (e.g., having an endothermic DSC event onset at 149-154° C.) to that depicted in FIG. 14B. In some embodiments, the present disclosure provides compound 15, wherein said compound has a DSC event and/or a TGA weight loss as described in Table 6.

In some embodiments, the present disclosure provides a composition comprising compound 15 and a pharmaceutically acceptable carrier or excipient.

In some embodiments, the present disclosure provides a method of modulating IRAK4 kinase in a patient comprising administering to said patient compound 15 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 15 or a composition thereof. In some embodiments, the present disclosure provides a method of treating a disease, disorder or condition in a patient, comprising administering to said patient compound 15 or a composition thereof, wherein the disease, disorder or condition is mediated by IRAK4. In some embodiments, the present disclosure provides a method of treating an autoimmune or inflammatory disorder in a patient, comprising administering to said patient compound 15 or a composition thereof.

### Compound 16 (Succinic Acid Salts of Compound 1)

According to one embodiment, the present disclosure provides a succinic acid salt of compound 1, represented by compound 16:

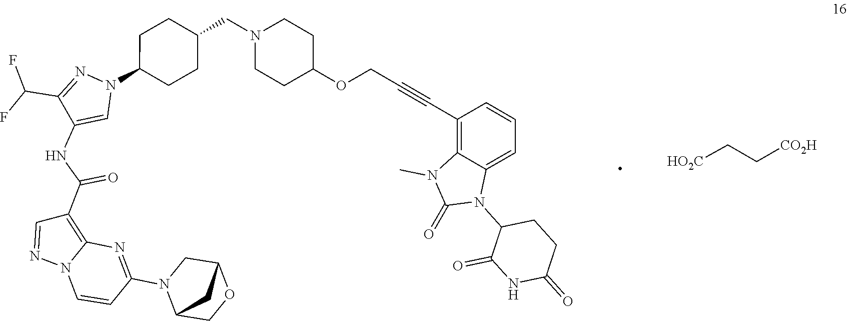 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CCC(=O)O]

It will be appreciated by one of ordinary skill in the art that the succinic acid and compound 1 are ionically bonded to form compound 16. In some embodiments, compound 16 is a hydrogen succinate salt. In some embodiments, compound 16 is a succinate salt. It is contemplated that compound 16 can exist in a variety of physical forms. For example, compound 16 can be in solution, suspension, or in solid form. In certain embodiments, compound 16 is in solid form. When compound 15 is in solid form, said compound may be amorphous, crystalline, or a mixture thereof. Exemplary solid forms are described in more detail below.

In some embodiments, the present disclosure provides compound 16 substantially free of impurities. Such impurities or extraneous matter may include different forms of compound 16, residual solvents, or any other impurities that may result from the preparation of, and/or isolation of, compound 16. In certain embodiments, at least about 95% by weight of a form of compound 16 is present. In still other embodiments of the disclosure, at least about 99% by weight of a form of compound 16 is present.

According to one embodiment, a form of compound 16 is present in an amount of at least about 97, 97.5, 98.0, 98.5, 99, 99.5, 99.8 weight percent where the percentages are based on the total weight of the composition. According to another embodiment, a form of compound 16 contains no more than about 3.0 area percent HPLC of total organic impurities and, in certain embodiments, no more than about 1.5 area percent HPLC total organic impurities relative to the total area of the HPLC chromatogram. In other embodiments, a form of compound 16 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 16 is also meant to include all tautomeric forms of compound 16. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 16 can exist in a variety of solid forms. Exemplary such forms include polymorphs such as those described herein.

In certain embodiments, compound 16 is a crystalline solid. In other embodiments, compound 16 is a crystalline solid substantially free of amorphous compound 16. As used herein, the term “substantially free of amorphous compound 16” means that the compound contains no significant amount of amorphous compound 16. In certain embodiments, at least about 95% by weight of crystalline compound 16 is present. In still other embodiments of the disclosure, at least about 99% by weight of crystalline compound 16 is present.

It has been found that compound 16 can exist in at least one distinct polymorphic form. In certain embodiments, the present disclosure provides a polymorphic form of compound 16 referred to herein as Form A.

### Form A of Compound 16

Methods for preparing Form A of compound 16 are described infra.

In some embodiments, the disclosure provides compound 16:

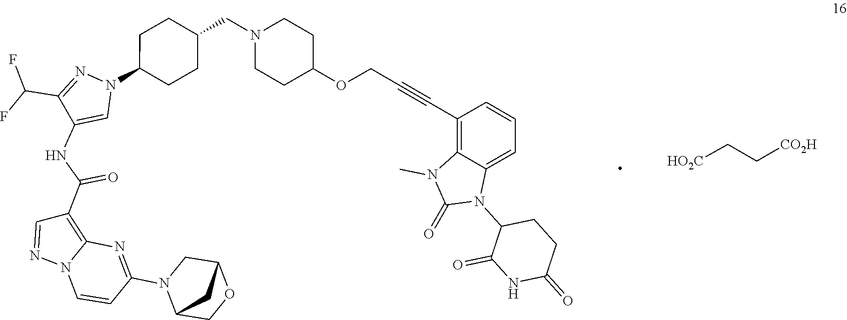 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CCC(=O)O]

wherein said compound is crystalline. In some embodiments, the present disclosure provides compound 16, wherein said compound is substantially free of amorphous compound 16. In some embodiments, the present disclosure provides compound 16, wherein said compound is substantially free of amorphous compound 1. In some embodiments, the present disclosure provides compound 16, wherein said compound is substantially free of a crystalline form of compound 1. In some embodiments, the present disclosure provides compound 16, wherein said compound is substantially free of impurities.

In some embodiments, Form A of compound 16 is characterized by one or more peaks in its XRPD pattern selected from those at 6.5, 12.9, and 16.2±0.5 degree 2-theta. In some embodiments, Form A of compound 16 is characterized by one or more peaks in its XRPD pattern selected from those at 6.5, 12.9, and 16.2±0.4 degree 2-theta. In some embodiments, Form A of compound 16 is characterized by one or more peaks in its XRPD pattern selected from those at 6.5, 12.9, and 16.2±0.3 degree 2-theta. In some embodiments, Form A of compound 16 is characterized by one or more peaks in its XRPD pattern selected from those at 6.5, 12.9, and 16.2±0.2 degree 2-theta.

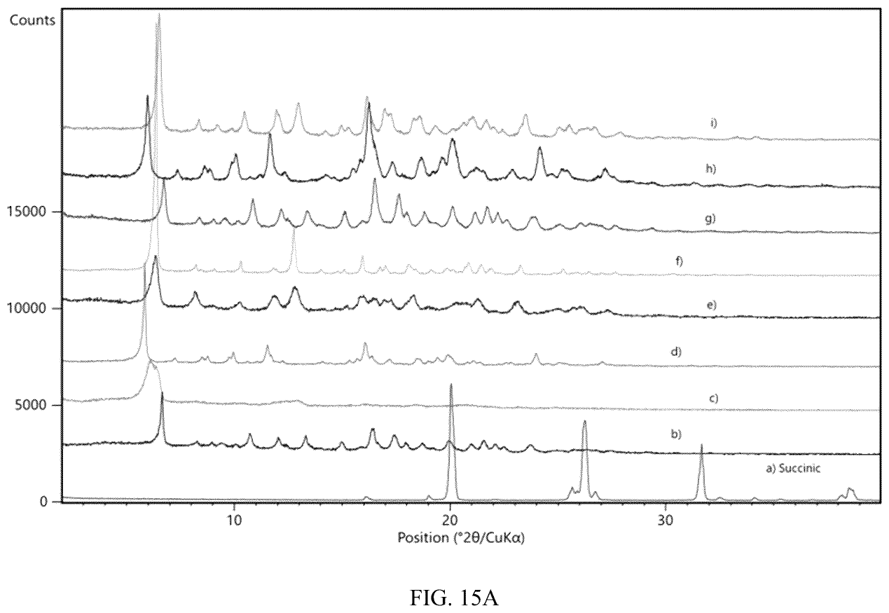

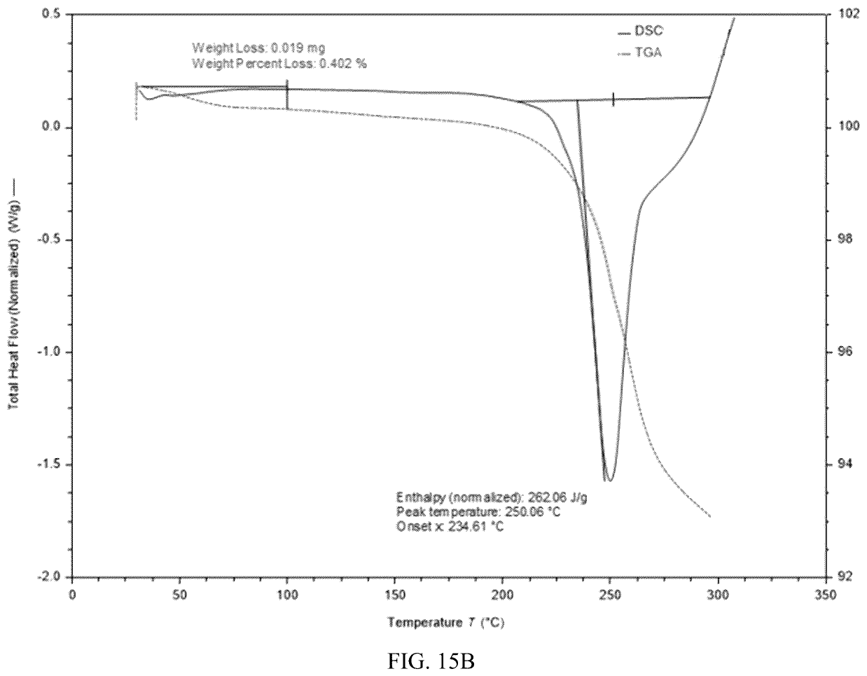

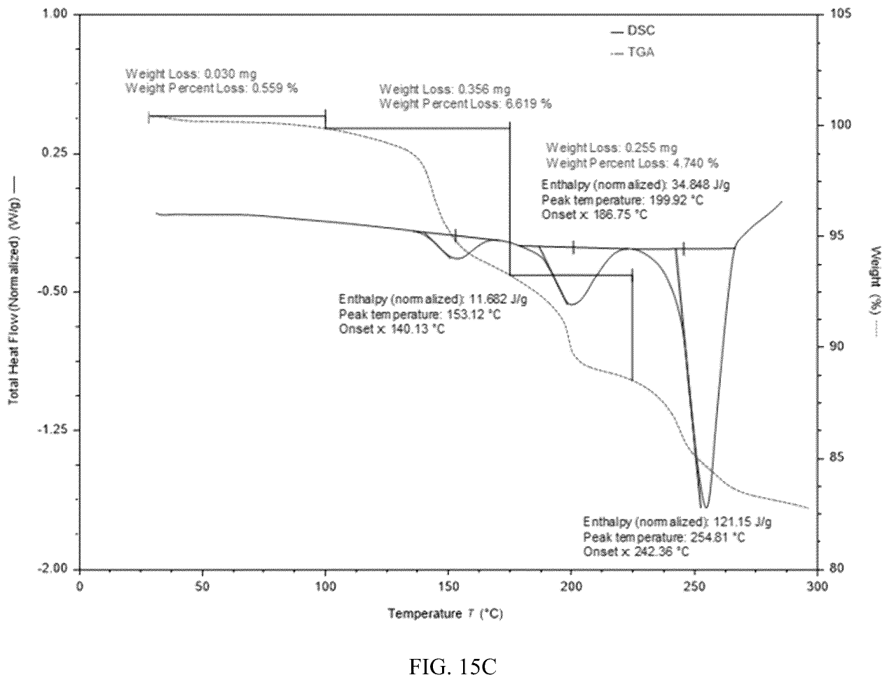

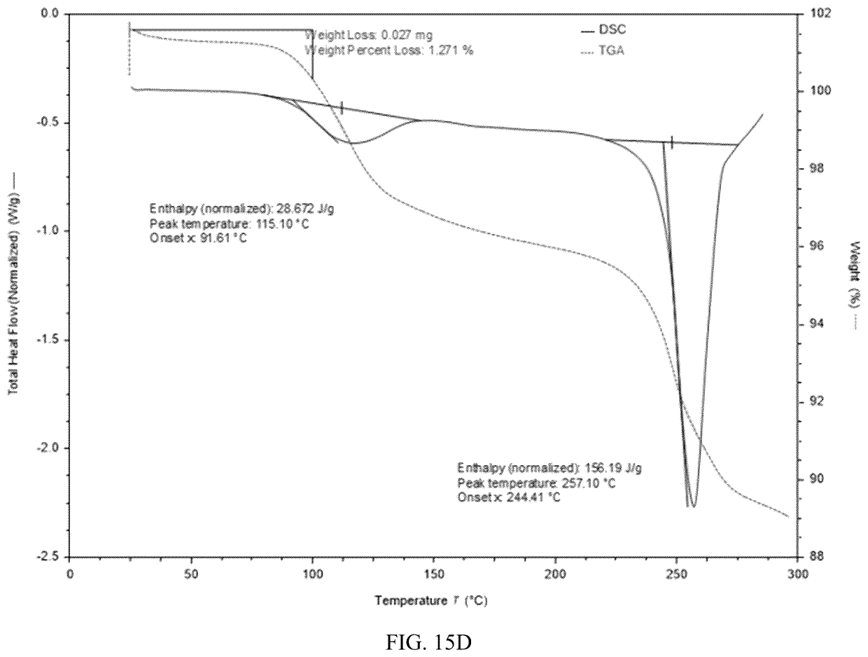

In some embodiments, the present disclosure provides compound 16, wherein said compound has an XRPD substantially similar (e.g., having peaks ±0.2 degree 2-theta) to that depicted in FIG. 15A.

In some embodiments, the present disclosure provides compound 16, wherein said compound has a DSC-TGA substantially similar (e.g., having an endothermic DSC event onset at 242-247° C.) to that depicted in FIG. 15D. In some embodiments, the present disclosure provides compound 16, wherein said compound has a DSC event and/or a TGA weight loss as described in Table 6.

In some embodiments, the present disclosure provides a composition comprising compound 16 and a pharmaceutically acceptable carrier or excipient.

In some embodiments, the present disclosure provides a method of modulating IRAK4 kinase in a patient comprising administering to said patient compound 16 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 16 or a composition thereof. In some embodiments, the present disclosure provides a method of treating a disease, disorder or condition in a patient, comprising administering to said patient compound 16 or a composition thereof, wherein the disease, disorder or condition is mediated by IRAK4. In some embodiments, the present disclosure provides a method of treating an autoimmune or inflammatory disorder in a patient, comprising administering to said patient compound 16 or a composition thereof.

### Form B of Compound 16

Methods for preparing Form B of compound 16 are described infra.

In some embodiments, the disclosure provides compound 16:

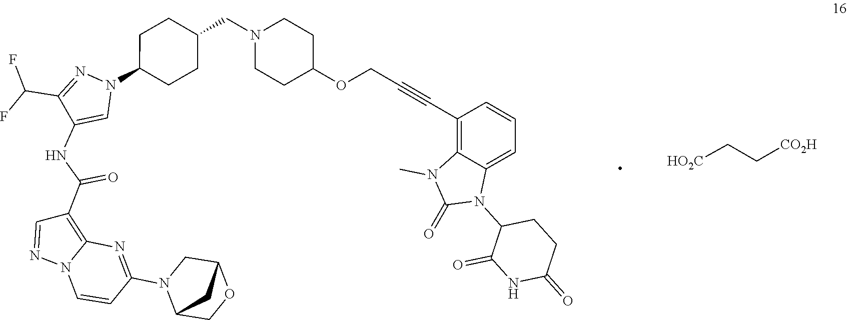 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CCC(=O)O]

wherein said compound is crystalline. In some embodiments, the present disclosure provides compound 16, wherein said compound is substantially free of amorphous compound 16. In some embodiments, the present disclosure provides compound 16, wherein said compound is substantially free of amorphous compound 1. In some embodiments, the present disclosure provides compound 16, wherein said compound is substantially free of a crystalline form of compound 1. In some embodiments, the present disclosure provides compound 16, wherein said compound is substantially free of impurities.

In some embodiments, Form B of compound 16 is characterized by one or more peaks in its XRPD pattern selected from those at 6.8, 11.1, and 16.5±0.5 degree 2-theta. In some embodiments, Form B of compound 16 is characterized by one or more peaks in its XRPD pattern selected from those at 6.8, 11.1, and 16.5±0.4 degree 2-theta. In some embodiments, Form B of compound 16 is characterized by one or more peaks in its XRPD pattern selected from those at 6.8, 11.1, and 16.5±0.3 degree 2-theta. In some embodiments, Form B of compound 16 is characterized by one or more peaks in its XRPD pattern selected from those at 6.8, 11.1, and 16.5±0.2 degree 2-theta.

In some embodiments, the present disclosure provides compound 16, wherein said compound has an XRPD substantially similar (e.g., having peaks ±0.2 degree 2-theta) to that depicted in FIG. 15A.

In some embodiments, the present disclosure provides compound 16, wherein said compound has a DSC-TGA substantially similar (e.g., having an endothermic DSC event onset at 232-237° C.) to that depicted in FIG. 15B. In some embodiments, the present disclosure provides compound 16, wherein said compound has a DSC event and/or a TGA weight loss as described in Table 6.

In some embodiments, the present disclosure provides a composition comprising compound 16 and a pharmaceutically acceptable carrier or excipient.

In some embodiments, the present disclosure provides a method of modulating IRAK4 kinase in a patient comprising administering to said patient compound 16 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 16 or a composition thereof. In some embodiments, the present disclosure provides a method of treating a disease, disorder or condition in a patient, comprising administering to said patient compound 16 or a composition thereof, wherein the disease, disorder or condition is mediated by IRAK4. In some embodiments, the present disclosure provides a method of treating an autoimmune or inflammatory disorder in a patient, comprising administering to said patient compound 16 or a composition thereof.

### Form C of Compound 16

Methods for preparing Form C of compound 16 are described infra.

In some embodiments, the disclosure provides compound 16:

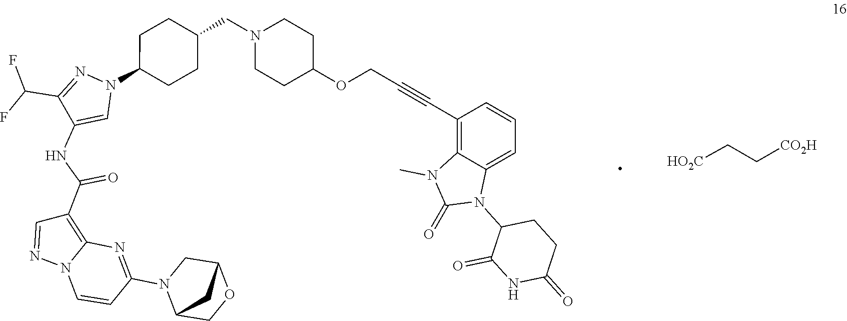 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; O=C(O)CCC(=O)O]

wherein said compound is crystalline. In some embodiments, the present disclosure provides compound 16, wherein said compound is substantially free of amorphous compound 16. In some embodiments, the present disclosure provides compound 16, wherein said compound is substantially free of amorphous compound 1. In some embodiments, the present disclosure provides compound 16, wherein said compound is substantially free of a crystalline form of compound 1. In some embodiments, the present disclosure provides compound 16, wherein said compound is substantially free of impurities.

In some embodiments, Form C of compound 16 is characterized by one or more peaks in its XRPD pattern selected from those at 6.0, 11.9, and 16.0±0.5 degree 2-theta. In some embodiments, Form C of compound 16 is characterized by one or more peaks in its XRPD pattern selected from those at 6.0, 11.9, and 16.0±0.4 degree 2-theta. In some embodiments, Form C of compound 16 is characterized by one or more peaks in its XRPD pattern selected from those at 6.0, 11.9, and 16.0±0.3 degree 2-theta. In some embodiments, Form C of compound 16 is characterized by one or more peaks in its XRPD pattern selected from those at 6.0, 11.9, and 16.0±0.2 degree 2-theta.

In some embodiments, the present disclosure provides compound 16, wherein said compound has an XRPD substantially similar (e.g., having peaks ±0.2 degree 2-theta) to that depicted in FIG. 15A.

In some embodiments, the present disclosure provides compound 16, wherein said compound has a DSC-TGA substantially similar (e.g., having an endothermic DSC event onset at 240-245° C.) to that depicted in FIG. 15C. In some embodiments, the present disclosure provides compound 16, wherein said compound has a DSC event and/or a TGA weight loss as described in Table 6.

In some embodiments, the present disclosure provides a composition comprising compound 16 and a pharmaceutically acceptable carrier or excipient.

In some embodiments, the present disclosure provides a method of modulating IRAK4 kinase in a patient comprising administering to said patient compound 16 or a composition thereof. In some embodiments, the method ubiquinates and/or degrades IRAK4 kinase, thereby treating a disease, disorder or condition associated with regulation of signaling pathways implicating IRAK4.

In some embodiments, the present disclosure provides a method of treating a disease, disorder or condition associated with regulation of signaling pathways implicating IRAK4 in a patient, comprising administering to said patient compound 16 or a composition thereof. In some embodiments, the present disclosure provides a method of treating a disease, disorder or condition in a patient, comprising administering to said patient compound 16 or a composition thereof, wherein the disease, disorder or condition is mediated by IRAK4. In some embodiments, the present disclosure provides a method of treating an autoimmune or inflammatory disorder in a patient, comprising administering to said patient compound 16 or a composition thereof.

### General Methods of Providing a Salt Compound

Salt compounds of general formula A, which formula encompasses, inter alia, salt compounds 2 through 16, and/or particular forms thereof, are prepared from compound 1, according to the general Scheme below.

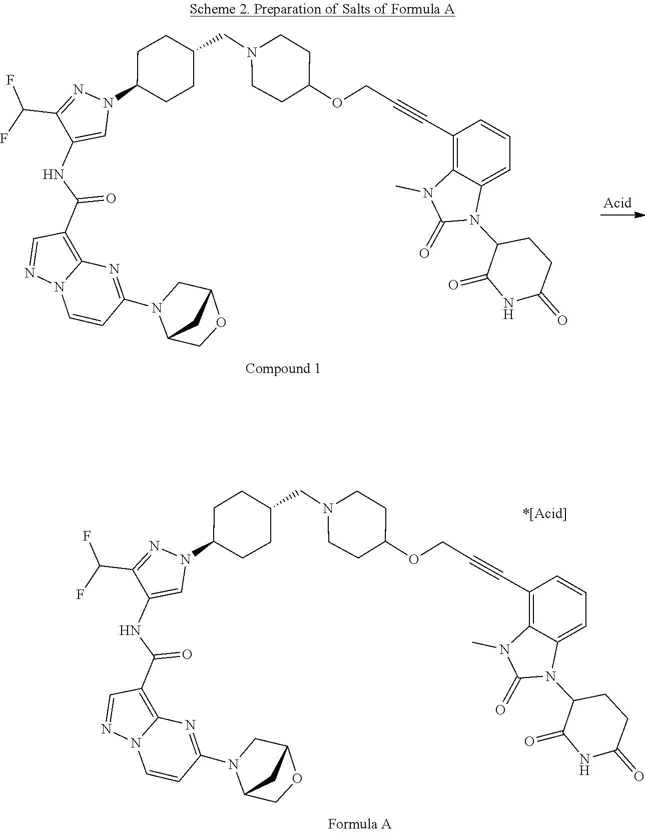

For instance, each of compounds 2 through 16, and forms thereof, are prepared from compound 1 by combining compound 1 with an appropriate acid to form a salt of that acid. Thus, another aspect of the present disclosure provides a method for preparing compounds 2 through 16, and forms thereof.

As described generally above, in some embodiments, the present disclosure provides a method for preparing a salt compound of the general formula A:

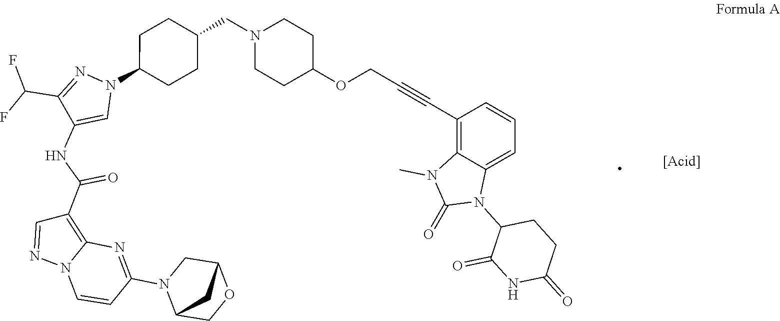

comprising steps of:
combining compound 1:

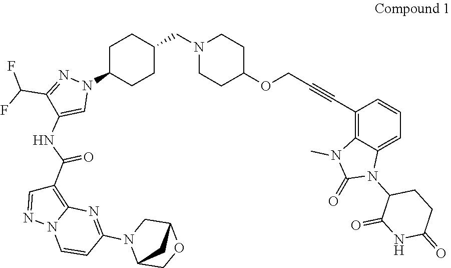 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

with a suitable acid and optionally a suitable solvent under conditions suitable for forming a salt of formula A.

In some embodiments, a suitable acid is sulfuric acid. In some embodiments, the present disclosure provides a method of making a sulfuric acid salt of compound 1. In certain embodiments, the sulfuric acid salt of compound 1 is compound 2. In certain embodiments, the sulfuric acid salt of compound 1 is Form A of compound 2. In some embodiments, the present invention provides a process of preparing Form A of compound 2 as described in Example 1, below.

In some embodiments, a suitable acid is benzenesulfonic acid. In some embodiments, the present disclosure provides a method of making a benzenesulfonic acid salt of compound 1. In certain embodiments, the benzenesulfonic acid salt of compound 1 is compound 3. In certain embodiments, the benzenesulfonic acid salt of compound 1 is Form A of compound 3. In some embodiments, the present invention provides a process of preparing Form A of compound 3 as described in Example 1, below.

In some embodiments, a suitable acid is toluenesulfonic acid. In some embodiments, the present disclosure provides a method of making a toluenesulfonic acid salt of compound 1. In certain embodiments, the toluenesulfonic acid salt of compound 1 is compound 4. In certain embodiments, the toluenesulfonic acid salt of compound 1 is Form A of compound 4. In some embodiments, the present invention provides a process of preparing Form A of compound 4 as described in Example 1, below.

In some embodiments, a suitable acid is ethanesulfonic acid. In some embodiments, the present disclosure provides a method of making a ethanesulfonic acid salt of compound 1. In certain embodiments, the ethanesulfonic acid salt of compound 1 is compound 5. In certain embodiments, the ethanesulfonic acid salt of compound 1 is Form A of compound 5. In some embodiments, the present invention provides a process of preparing Form A of compound 5 as described in Example 1, below.

In some embodiments, a suitable acid is methanesulfonic acid. In some embodiments, the present disclosure provides a method of making a methanesulfonic acid salt of compound 1. In certain embodiments, the methanesulfonic acid salt of compound 1 is compound 6. In certain embodiments, the methanesulfonic acid salt of compound 1 is Form A of compound 6. In some embodiments, the present invention provides a process of preparing Form A of compound 6 as described in Example 1, below.

In some embodiments, a suitable acid is maleic acid. In some embodiments, the present disclosure provides a method of making a maleic acid salt of compound 1. In certain embodiments, the maleic acid salt of compound 1 is compound 7. In certain embodiments, the maleic acid salt of compound 1 is Form A of compound 7. In some embodiments, the present invention provides a process of preparing Form A of compound 7 as described in Example 1, below.

In some embodiments, a suitable acid is L-tartaric acid. In some embodiments, the present disclosure provides a method of making a L-tartaric acid salt of compound 1. In certain embodiments, the L-tartaric acid salt of compound 1 is compound 8. In certain embodiments, the L-tartaric acid salt of compound 1 is Form A of compound 8. In some embodiments, the present invention provides a process of preparing Form A of compound 8 as described in Example 1, below.

In some embodiments, a suitable acid is fumaric acid. In some embodiments, the present disclosure provides a method of making a fumaric acid salt of compound 1. In certain embodiments, the fumaric acid salt of compound 1 is compound 9. In certain embodiments, the fumaric acid salt of compound 1 is Form A of compound 9. In some embodiments, the present invention provides a process of preparing Form A of compound 9 as described in Example 1, below.

In some embodiments, a suitable acid is citric acid. In some embodiments, the present disclosure provides a method of making a citric acid salt of compound 1. In certain embodiments, the citric acid salt of compound 1 is compound 10. In certain embodiments, the citric acid salt of compound 1 is Form A of compound 10. In some embodiments, the present invention provides a process of preparing Form A of compound 10 as described in Example 1, below.

In some embodiments, a suitable acid is glycolic acid. In some embodiments, the present disclosure provides a method of making a glycolic acid salt of compound 1. In certain embodiments, the glycolic acid salt of compound 1 is compound 11. In certain embodiments, the glycolic acid salt of compound 1 is Form A of compound 11. In some embodiments, the present invention provides a process of preparing Form A of compound 11 as described in Example 1, below.

In some embodiments, a suitable acid is L-malic acid. In some embodiments, the present disclosure provides a method of making a L-malic acid salt of compound 1. In certain embodiments, the L-malic acid salt of compound 1 is compound 12. In certain embodiments, the L-malic acid salt of compound 1 is Form A of compound 12. In some embodiments, the present invention provides a process of preparing Form A of compound 12 as described in Example 1, below.

In some embodiments, a suitable acid is hippuric acid. In some embodiments, the present disclosure provides a method of making a hippuric acid salt of compound 1. In certain embodiments, the hippuric acid salt of compound 1 is compound 13. In certain embodiments, the hippuric acid salt of compound 1 is Form A of compound 13. In some embodiments, the present invention provides a process of preparing Form A of compound 13 as described in Example 1, below.

In some embodiments, a suitable acid is L-lactic acid. In some embodiments, the present disclosure provides a method of making a L-lactic acid salt of compound 1. In certain embodiments, the L-lactic acid salt of compound 1 is compound 14. In certain embodiments, the L-lactic acid salt of compound 1 is Form A of compound 14. In some embodiments, the present invention provides a process of preparing Form A of compound 14 as described in Example 1, below.

In some embodiments, a suitable acid is benzoic acid. In some embodiments, the present disclosure provides a method of making a benzoic acid salt of compound 1. In certain embodiments, the benzoic acid salt of compound 1 is compound 15. In certain embodiments, the benzoic acid salt of compound 1 is Form A of compound 15. In some embodiments, the present invention provides a process of preparing Form A of compound 15 as described in Example 1, below.

In some embodiments, a suitable acid is succinic acid. In some embodiments, the present disclosure provides a method of making a succinic acid salt of compound 1. In certain embodiments, the succinic acid salt of compound 1 is compound 16. In certain embodiments, the succinic acid salt of compound 1 is Form A of compound 16. In some embodiments, the present invention provides a process of preparing Form A of compound 16 as described in Example 1, below. In certain embodiments, the succinic acid salt of compound 1 is Form B of compound 16. In some embodiments, the present invention provides a process of preparing Form B of compound 16 as described in Example 1, below. In certain embodiments, the succinic acid salt of compound 1 is Form C of compound 16. In some embodiments, the present invention provides a process of preparing Form C of compound 16 as described in Example 1, below.

A suitable solvent may be any solvent system (e.g., one solvent or a mixture of solvents) in which compound 1 and/or an acid are soluble, or are at least partially soluble.

Examples of suitable solvents useful in the presently disclosed methods include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, the solvent is one or more organic alcohols. In some embodiments, the solvent is chlorinated. In some embodiments, the solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, ethyl acetate, dimethyl formamide, dimethylsulfoxide, glyme, diglyme, methyl t-butyl ether, t-butanol, n-butanol, and acetonitrile. In certain embodiments, a suitable solvent is acetonitrile, ethyl acetate, tetrahydrofuran, methanol, or 9:1 acetone:water.

In some embodiments, a suitable solvent is ethyl acetate. In some embodiments, a suitable solvent is methanol. In some embodiments, a suitable solvent is acetonitrile. In some embodiments, a suitable solvent is tetrahydrofuran. In some embodiments, a suitable solvent is 9:1 acetone:water.

In some embodiments, the present disclosure provides a method for preparing a salt compound of the general formula A, comprising one or more steps of removing a solvent and adding a solvent. In some embodiments, an added solvent is the same as the solvent removed. In some embodiments, an added solvent is different from the solvent removed. Means of solvent removal are known in the synthetic and chemical arts and include, but are not limited to, any of those described herein and in the Examples.

In some embodiments, a method for preparing a salt compound of the general formula A comprises one or more steps of heating or cooling a preparation.

In some embodiments, a method for preparing a salt compound of the general formula A comprises one or more steps of agitating or stirring a preparation.

In some embodiments, a method for preparing a salt compound of the general formula A comprises mixing compound 1 and suitable solvent and agitating or stirring the mixture at a certain temperature for an amount of time. In some embodiments, the method comprises mixing compound 1 and a suitable solvent in a 1:1 molar ratio. In some embodiments, the method comprises agitating or stirring the mixture at room temperature. In some embodiments, the method comprises agitating or stirring the mixture for 1, 2, 3, or 4 days.

In some embodiments, a method for preparing a salt compound of the general formula A comprises slow evaporation of the solvent. In some embodiments, a method for preparing a salt compound of the general formula A comprises slow evaporation of the solvent through exposure to ambient atmosphere at room temperature. In some embodiments, a method for preparing a salt compound of the general formula A comprises evaporation of the solvent under a flow of an inert gas, e.g., nitrogen gas.

In some embodiments, a method for preparing a salt compound of the general formula A comprises a step of adding a suitable acid to a solution or slurry of compound 1.

In some embodiments, a method for preparing a salt compound of the general formula A comprises a step of heating.

In certain embodiments, a salt compound of formula A precipitates from the mixture. In another embodiment, a salt compound of formula A crystallizes from the mixture. In other embodiments, a salt compound of formula A crystallizes from solution following seeding of the solution (i.e., adding crystals of a salt compound of formula A to the solution).

A salt compound of formula A can precipitate out of the reaction mixture, or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a salt compound of formula A is optionally isolated. It will be appreciated that a salt compound of formula A may be isolated by any suitable physical means known to one of ordinary skill in the art. In certain embodiments, precipitated solid salt compound of formula A is separated from the supernatant by filtration. In other embodiments, precipitated solid salt compound of formula A is separated from the supernatant by decanting the supernatant.

In certain embodiments, a salt compound of formula A is separated from the supernatant by filtration.

In certain embodiments, an isolated salt compound of formula A is dried in air. In other embodiments an isolated salt compound of formula A is dried under reduced pressure, optionally at elevated temperature.

### Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit an IRAK protein kinase, or a mutant thereof, in a biological sample or in a patient. In certain embodiments, the amount of compound in compositions of this invention is such that is effective to measurably degrade and/or inhibit an IRAK protein kinase, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of an IRAK protein kinase, or a mutant thereof.

As used herein, the term “degratorily active metabolite or residue thereof” means that a metabolite or residue thereof is also a degrader of an IRAK protein kinase, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of kinase activity of one or more enzymes.

Examples of kinases that are degraded and/or inhibited by the compounds and compositions described herein and against which the methods described herein are useful include those of the interleukin-1 receptor-associated kinase (IRAK) family of kinases, the members of which include IRAK-1, IRAK-2, and IRAK-4, or a mutant thereof. Li et al., “IRAK-4: A novel member of the IRAK family with the properties of an IRAK-kinase,” PNAS 2002, 99(8), 5567-5572, Flannery et al., “The interleukin-1 receptor-associated kinases: Critical regulators of innate immune signaling” Biochem Pharm 2010, 80(12), 1981-1991 incorporated by reference in its entirety.

According to one embodiment, the invention relates to a method of inhibiting protein kinase activity or degading a protein kinase in a biological sample comprising the step of contacting said biological sample with a compound of this invention, or a composition comprising said compound.

According to another embodiment, the invention relates to a method of inhibiting or degrading IRAK-1, IRAK-2, and/or IRAK-4, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition and/or degradation of a protein kinase, or a protein kinase selected from IRAK-1, IRAK-2, and/or IRAK-4, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting one or more of IRAK-1, IRAK-2, and/or IRAK-4, or a mutant thereof, activity in a patient comprising the step of administering to said patient a compound of the present invention, or a composition comprising said compound. In other embodiments, the present invention provides a method for treating a disorder mediated by one or more of IRAK-1, IRAK-2, and/or IRAK-4, or a mutant thereof, in a patient in need thereof, comprising the step of administering to said patient a compound according to the present invention or pharmaceutically acceptable composition thereof. Such disorders are described in detail herein.

The activity of a compound utilized in this invention as a degrader and/or inhibitor of IRAK-1, IRAK-2, and/or IRAK-4, or a mutant thereof, may be assayed in vitro, in vivo or in a cell line. In vitro assays include assays that determine inhibition of either the phosphorylation activity and/or the subsequent functional consequences, or ATPase activity of activated IRAK-1, IRAK-2, and/or IRAK-4, or a mutant thereof. Alternate in vitro assays quantitate the ability of the inhibitor to bind to IRAK-1, IRAK-2 and/or IRAK-4. Inhibitor binding may be measured by radiolabeling the inhibitor prior to binding, isolating the inhibitor/IRAK-1, inhibitor/IRAK-2, or inhibitor/IRAK-4 complex and determining the amount of radiolabel bound. Alternatively, inhibitor binding may be determined by running a competition experiment where new inhibitors are incubated with IRAK-1, IRAK-2, and/or IRAK-4 bound to known radioligands. Representative in vitro and in vivo assays useful in assaying an IRAK-4 inhibitor include those described and disclosed in, e.g., Kim et al., “A critical role for IRAK4 kinase activity in Toll-like receptor-mediated innate immunity,” J. Exp. Med. 2007 204(5), 1025-1036; Lebakken et al., “A Fluorescence Lifetime Based Binding Assay to Characterize Kinase Inhibitors,” J. Biomol. Screen. 2007, 12(6), 828-841; Maschera et al., “Overexpression of an enzymatically inactive interleukin-1-receptor-associated kinase activates nuclear factor-κB,” Biochem. J. 1999, 339, 227-231; Song et al., “The kinase activities of interleukin-e receptor associated kinase (IRAK)-1 and 4 are redundant in the control of inflammatory cytokine expression in human cells,” Mol. Immunol. 2009, 46, 1458-1466, the entirety of each of which is herein incorporated by reference. Detailed conditions for assaying a compound utilized in this invention as a degrader and/or inhibitor of IRAK-1, IRAK-2, and/or IRAK-4, or a mutant thereof, are set forth in the Examples below.

The best characterized member of the IRAK family is the serine/threonine kinase IRAK-4. IRAK-4 is implicated in signaling innate immune responses from Toll-like receptors (TLRs) and Toll/IL-1 receptors (TIRs).

Innate immunity detects pathogens through the recognition of pathogen-associated molecular patterns by TLRs, when then links to the adaptive immune response. TLRs recognize conserved structures of both microbes and endogenous molecules. TLRs which recognize bacterial and fungal components are located on the cell surface, whereas TLRs which recognize viral or microbial nucleic acids are localized to intracellular membranes such as endosomes and phagosomes. Cell surface TLRs can be targeted by small molecules and antibodies, whereas intracellular TLRs require targeting with oligonucleotides.

TLRs mediate the innate immune response by upregulating the expression of inflammatory genes in multiple target cells. See. e.g., Sen et al., “Transcriptional signaling by double-stranded RNA: role of TLR3,” Cytokine & Growth Factor Rev. 2005, 16, 1-14, incorporated by reference in its entirety. While TLR-mediated inflammatory response is critical for innate immunity and host defense against infections, uncontrolled inflammation is detrimental to the host leading to sepsis and chronic inflammatory diseases, such as chronic arthritis, atherosclerosis, multiple sclerosis, cancers, autoimmune disorders such as rheumatoid arthritis, lupus, asthma, psoriasis, and inflammatory bowel diseases.

Upon binding of a ligand, most TLRs recruit the adaptor molecule MyD88 through the TIR domain, mediating the MyD88-dependent pathway. MyD88 then recruits IRAK-4, which engages with the nuclear factor-κB (NF-κB), mitogen-activated protein (MAP) kinase and interferon-regulatory factor cascades and leads to the induction of pro-inflammatory cytokines. The activation of NF-κB results in the induction of inflammatory cytokines and chemokines, such as TNF-α, IL-1α, IL-6 and IL-8. The kinase activity of IRAK-4 has been shown to play a critical role in the TLR-mediated immune and inflammatory responses. IRAK4 is a key mediator of the innate immune response orchestrated by interleukin-1 receptor (IL-1R), interleukin-18 receptor (IL-18R), IL-33 receptor (IL-33R), and Toll-like receptors (TLRs). Inactivation of IRAK-1 and/or IRAK-4 activity has been shown to result in diminished production of cytokines and chemokines in response to stimulation of IL-1 and TLR ligands. See, e.g., Picard et al., “Clinical features and outcome of patients with IRAK-4 and MyD88 deficiency,” Medicine (Baltimore), 2010, 89(6), 043-25; Li, “IRAK4 in TLR/IL-1R signaling: Possible clinical applications,” Eur. J. Immunology 2008, 38:614-618; Cohen et al., “Targeting protein kinases for the development of anti-inflammatory drugs,” Curr. Opin. Cell Bio. 2009, 21:317-324; Flannery et al., “The interleukin-1 receptor-associated kinases: Critical regulators of innate immune signalling,” Biochem. Pharm. 2010, 80(12), 1981-1991; Gottipati et al., “IRAK1: A critical signaling mediator of innate immunity,” Cellular Signaling 2008, 20, 269-276; Kim et al., “A critical role for IRAK4 kinase activity in Toll-like receptor-mediated innate immunity,” J. Exp. Med. 2007 204(5), 1025-1036; Koziczak-Holbro et al., “IRAK-4 Kinase Activity Is Required for Interleukin-1 (IL-1) Receptor- and Toll-like Receptor 7-mediated Signaling and Gene Expression,” J. Biol. Chem. 2007, 282(18), 13552-13560; Kubo-Murai et al., “IRAK-4-dependent Degradation of IRAK-1 is a Negative Feedback Signal for TLR-mediated NF-×B Activation,” J. Biochem. 2008, 143, 295-302; Maschera et al., “Overexpression of an enzymatically inactive interleukin-1-receptor-associated kinase activates nuclear factor-κB,” Biochem. J. 1999, 339, 227-231; Lin et al., “Helical assembly in the MyD88-IRAK4-IRAK2 complex in TLR/IL-1R signalling,” Nature 2010, 465(17), 885-891; Suzuki et al., “IRAK-4 as the central TIR signaling mediator in innate immunity,” TRENDS in Immunol. 2002, 23(10), 503-506; Suzuki et al., “Severe impairment of interleukin-1 and Toll-like receptor signalling in mice lacking IRAK-4,” Nature 2002, 416, 750-754; Swantek et al., “IL-1 Receptor-Associated Kinase Modulates Host Responsiveness to Endotoxin,” J. Immunol. 2000, 164, 4301-4306; Hennessy, E., et al., “Targeting Toll-like receptors: emerging therapeutics?” Nature Reviews, vol. 9, pp: 293-307 (2010); Dinarello, C. “Interleukin-18 and the Pathogenesis of Inflammatory Diseases,” Seminars in Nephrology, vol. 27, no. 1, pp: 98-114 (2007), the entirety of each of which is herein incorporated by reference. In fact, knockdown mice that express a catalytically inactive mutant IRAK-4 protein are completely resistant to septic shock and show impaired IL-1 activity. Moreover, these mice are resistant to joint and bone inflammation/destruction in an arthritis model, suggesting that IRAK-4 may be targeted to treat chronic inflammation. Further, while IRAK-4 appears to be vital for childhood immunity against some pyogenic bacteria, it has been shown to play a redundant role in protective immunity to most infections in adults, as demonstrated by one study in which patients older than 14 lacking IRAK-4 activity exhibited no invasive infections. Cohen et al., “Targeting protein kinases for the development of anti-inflammatory drugs,” Curr. Opin. Cell Bio. 2009, 21:317-324; Ku et al., “Selective predisposition to bacterial infections in IRAK-4-deficient children: IRAK-4-dependent TLRs are otherwise redundant in protective immunity,” J. Exp. Med. 2007, 204(10), 2407-2422; Picard et al., “Inherited human IRAK-4 deficiency: an update,” Immunol. Res. 2007, 38, 347-352; Song et al., “The kinase activities of interleukin-e receptor associated kinase (IRAK)-1 and 4 are redundant in the control of inflammatory cytokine expression in human cells,” Mol. Immunol. 2009, 46, 1458-1466; Rokosz, L. et al., “Kinase inhibitors as drugs for chronic inflammatory and immunological diseases: progress and challenges,” Expert Opinions on Therapeutic Targets, 12(7), pp: 883-903 (2008); Gearing, A. “Targeting toll-like receptors for drug development: a summary of commercial approaches,” Immunology and Cell Biology, 85, pp: 490-494 (2007); Dinarello, C. “IL-1: Discoveries, controversies and future directions,” European Journal of Immunology, 40, pp: 595-653 (2010), the entirety of each of which is herein incorporated by reference. Because TLR activation triggers IRAK-4 kinase activity, IRAK-4 inhibition presents an attractive target for treating the underlying causes of inflammation in countless diseases.

Representative IRAK-4 inhibitors include those described and disclosed in e.g., Buckley et al., Bioorg. Med. Chem. Lett. 2008, 18, 3211-3214; Buckley et al., Bioorg. Med. Chem. Lett. 2008, 18, 3291-3295; Buckley et al., Bioorg. Med. Chem. Lett. 2008, 18, 3656-3660; Powers et al., “Discovery and initial SAR of inhibitors of interleukin-1 receptor-associated kinase-4,” Bioorg. Med. Chem. Lett. 2006, 16, 2842-2845; Wng et al., “IRAK-4 Inhibitors for Inflammation,” Curr. Topics in Med. Chem. 2009, 9, 724-737, the entirety of each of which is herein incorporated by reference.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of one of more of IRAK-1, IRAK-2, and/or IRAK-4 and are therefore useful for treating one or more disorders associated with activity of one or more of IRAK-1, IRAK-2, and/or IRAK-4. Thus, in certain embodiments, the present invention provides a method for treating an IRAK-1-mediated, an IRAK-2-mediated, and/or an IRAK-4-mediated disorder comprising the step of administering to a patient in need thereof a compound of the present invention, or pharmaceutically acceptable composition thereof.

As used herein, the terms “IRAK-1-mediated”, “IRAK-2-mediated”, and/or “IRAK-4-mediated” disorders, diseases, and/or conditions as used herein means any disease or other deleterious condition in which one or more of IRAK-1, IRAK-2, and/or IRAK-4, or a mutant thereof, are known to play a role. Accordingly, another embodiment of the present invention relates to treating or lessening the severity of one or more diseases in which one or more of IRAK-1, IRAK-2, and/or IRAK-4, or a mutant thereof, are known to play a role.

In some embodiments, compounds and compositions described herein are useful in medicine.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer (see, e.g., Ngo, V. et al., “Oncogenically active MYD88 mutations in human lymphoma,” Nature, vol. 000, pp: 1-7 (2010); Lust, J. et al., “Induction of a Chronic Disease State in patients With Smoldering of Indolent Multiple Myeloma by Targeting Interleukin 1ß-Induced Interleukin 6 Production and the Myeloma Proliferative Component,” Mayo Clinic Proceedings, 84(2), pp: 114-122 (2009)), diabetes, cardiovascular disease, viral disease, autoimmune diseases such as lupus (see, e.g., Dinarello, C. “Interleukin-18 and the Pathogenesis of Inflammatory Diseases,” Seminars in Nephrology, vol. 27, no. 1, pp: 98-114 (2007); Cohen et al., “Targeting protein kinases for the development of anti-inflammatory drugs,” Curr. Opin. Cell Bio. 2009, 21:317-324) and rheumatoid arthritis (see, e.g., Geyer, M. et al., “Actual status of antiinterleukin-1 therapies in rheumatic diseases,” Current Opinion in Rheumatology, 22, pp: 246-251 (2010)), autoinflammatory syndromes (see, e.g., Hoffman, H. et al., “Efficacy and Safety of Rilonacept (Interleukin-1 Trap) in Patients with Cryopyrin-Associated Periodic Syndromes,” Arthritis & Rheumatism, vol. 58, no. 8, pp: 2443-2452 (2008)), atherosclerosis, psoriasis, allergic disorders, inflammatory bowel disease (see, e.g., Cario, E. “Therapeutic Impact of Toll-like Receptors on Inflammatory Bowel Diseases: A Multiple-edged Sword,” Inflamm. Bowel Dis., 14, pp: 411-421 (2008)), inflammation (see, e.g., Dinarello, C. “Interleukin 1 and interleukin 18 as mediators of inflammation and the aging process,” The American Journal of Clinical Nutrition, 83, pp: 447S-455S (2006)), acute and chronic gout and gouty arthritis (see, e.g., Terkeltaub, R. “Update on gout: new therapeutic strategies and options,” Nature, vol. 6, pp: 30-38 (2010); Weaver, A. “Epidemiology of gout,” Cleveland Clinic Journal of Medicine, vol. 75, suppl. 5, pp: S9-S12 (2008); Dalbeth, N. et al., “Hyperuricaemia and gout: state of the art and future perspectives,” Annals of Rheumatic Diseases, 69, pp: 1738-1743 (2010); Martinon, F. et al., “Gout-associated uric acid crystals activate the NALP3 inflammasome,” Nature, vol. 440, pp: 237-241 (2006); So, A. et al., “A pilot study of IL-1 inhibition by anakinra in acute gout,” Arthritis Research & Therapy, vol. 9, no. 2, pp: 1-6 (2007); Terkeltaub, R. et al., “Ihe interleukin 1 inhibitor rilonacept in treatment of chronic gouty arthritis: results of a placebo-controlled, monosequence crossover, non-randomised, single-blind pilot study,” Annals of Rheumatic Diseases, 68, pp: 1613-1617 (2009); Torres, R. et al., “Hyperalgesia, synovitis and multiple biomarkers of inflammation are suppressed by interleukin 1 inhibition in a novel animal model of gouty arthritis,” Annals of Rheumatic Diseases, 68, pp: 1602-1608 (2009)), neurological disorders, metabolic syndrome (see. e.g., Troseid, M. “The role of interleukin-18 in the metabolic syndrome,” Cardiovascular Diabetology, 9:11, pp: 1-8 (2010)), immunodeficiency disorders such as AIDS and HIV (see, e.g., Iannello, A. et al., “Role of Interleukin-18 in the Development and Pathogenesis of AIDS,” AIDS Reviews, 11, pp: 115-125 (2009)), destructive bone disorders (see, e.g., Hennessy, E., et al., “Targeting Toll-like receptors: emerging therapeutics?” Nature Reviews, vol. 9, pp: 293-307 (2010)), osteoarthritis, proliferative disorders, Waldenström's Macroglobulinemia (see, e.g., Treon, et al., “Whole genome sequencing reveals a widely expressed mutation (MYD88 L265P) with oncogenic activity in Waldenström's Macroglobulinemia” 53^{rd} ASH Annual Meeting; Xu, et al., “A somatic variant in MYD88 (L256P) revealed by whole genome sequencing differentiates lymphoplasmacytic lymphoma from marginal zone lymphomas” 53^{rd} ASH Annual Meeting; Yang et al., “Disruption of MYD88 pathway signaling leads to loss of constitutive IRAK1, NK-kB and JAK/STAT signaling and induces apoptosis of cells expressing the MYD88 L265P mutation in Waldenström's Macroglobulinemia” 53^{rd} ASH Annual Meeting; Iriyama et al., “Clinical significance of genetic mutations of CD79B, CARD11, MYD88, and EZH2 genes in diffuse large B-cell lymphoma patients” 53^{rd} ASH Annual Meeting; infectious diseases, conditions associated with cell death, pathologic immune conditions involving T cell activation, and CNS disorders in a patient. In one embodiment, a human patient is treated with a compound of the current invention and a pharmaceutically acceptable carrier, adjuvant, or vehicle, wherein said compound is present in an amount to measurably degrade and/or inhibit IRAK-1 only, IRAK-2-only, IRAK-4-only and/or IRAK1 and IRAK4 kinase activity.

Compounds of the current invention are useful in the treatment of a proliferative disease selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenström's macroglobulinemia (WM), splenic marginal zone lymphoma, multiple myeloma, plasmacytoma, intravascular large B-cell lymphoma, AML, MDS).

In some embodiments the proliferative disease which can be treated according to the methods of this invention is a MyD88 driven disorder. In some embodiments, the MyD88 driven disorder which can be treated according to the methods of this invention is selected from ABC DLBCL, primary CNS lymphomas, primary extranodal lymphomas, Waldenstróm's macroglobulinemia, Hodgkin's lymphoma, primary cutaneous T-cell lymphoma and chronic lymphocytic leukemia.

In some embodiments the proliferative disease which can be treated according to the methods of this invention is an IL-1 driven disorder. In some embodiments the IL-1 driven disorder is smoldering multiple myeloma.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds according to the invention are useful in the treatment of heteroimmune diseases. Examples of such heteroimmune diseases include, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophilic asthma, eosinophilic COPD, and eosinophil-related disorders affecting the airways occasioned by drug-reaction.

Compounds of the invention are also useful in the treatment of inflammatory or allergic conditions of the skin, for example psoriasis, generalized pustular psoriasis (GPP), psoriasis vulgaris, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, hidradenitis suppurativa, Sweet Syndrome, pyoderma gangrenosum, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is a disease of the skin. In some embodiments, the inflammatory disease of the skin is selected from contact dermatitits, atompic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, hidradenitis suppurativa, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), Adult Onset Still's disease, macrophage activation syndrome (MAS), primary and secondary hemophagocytic lymphohistiocytosis (HLH), Familial Mediterranean Fever, NLRP12 autoinflammatory syndrome, and osteoarthritis.

In some embodiments the inflammatory disease which can be treated according to the methods of this invention is a TH17 mediated disease. In some embodiments the TH17 mediated disease is selected from Systemic lupus erythematosus, Multiple sclerosis, psoriasis vulgaris, hidradenitis suppurativa, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis or chronic rhinosinusitis with nasal polyps (CRSwNP).

In some embodiments, the present invention provides a method of treating a TLR/IL-1R-driven autoinflammatory and autoimmune diseases with high unmet medical need, such as hidradenitis suppurativa, atopic dermatitis and rheumatoid arthritis. As such, in some embodiments, the present invention provides a method of treating hidradenitis suppurativa in a patient in need thereof, comprising administrating a compound of the present invention, or a pharmaceutically acceptable salt thereof. In some embodiments, the present invention provides a method of treating atopic dermatitis in a patient in need thereof, comprising administrating a compound of the present invention, or a pharmaceutically acceptable salt thereof. In some embodiments, the present invention provides a method of treating rheumatoid arthritis in a patient in need thereof, comprising administrating a compound of the present invention, or a pharmaceutically acceptable salt thereof.

Cardiovascular diseases which can be treated according to the methods of this invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of this invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

The loss of IRAK4 function results in decreased Aβ levels in an in vivo murine model of Alzheimer's disease and was associated with diminished microgliosis and astrogliosis in aged mice. Analysis of microglia isolated from the adult mouse brain revealed an altered pattern of gene expression associated with changes in microglial phenotype that were associated with expression of IRF transcription factors that govern microglial phenotype. Further, loss of IRAK4 function also promoted amyloid clearance mechanisms, including elevated expression of insulin-degrading enzyme. Finally, blocking IRAK function restored olfactory behavior (Cameron et al. “Loss of Interleukin Receptor-Associated Kinase 4 Signaling Suppresses Amyloid Pathology and Alters Microglial Phenotype in a Mouse Model of Alzheimer's Disease” Journal of Neuroscience (2012) 32(43), 15112-15123).

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV infection.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound or a pharmaceutically acceptable salt thereof and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (TMeo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound or a pharmaceutically acceptable salt thereof and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound or a pharmaceutically acceptable salt thereof and one or more additional therapeutic agents selected from anti-IL-33 antibodies such as REGN3500 (SAR440340) or CNTO 7160, Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (1heo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound or a pharmaceutically acceptable salt thereof and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (TMeo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, In some embodiments, the present invention provides a method of treating eosinophilic COPD comprising administering to a patient in need thereof a provided compound or a pharmaceutically acceptable salt thereof and one or more additional therapeutic agents selected from an anti-IL-33 antibody such as REGN3500 (SAR440340) or CNTO 7160. In some embodiments, the present invention provides a method of treating eosinophilic asthma comprising administering to a patient in need thereof a provided compound or a pharmaceutically acceptable salt thereof and one or more additional therapeutic agents selected from an anti-IL-33 antibody such as REGN3500 (SAR440340) or CNTO 7160.

In some embodiments, the present invention provides a method of treating HIV comprising administering to a patient in need thereof a provided compound or a pharmaceutically acceptable salt thereof and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound or a pharmaceutically acceptable salt thereof and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In some embodiments, the present invention provides a method of treating DLBCL comprising administering to a patient in need thereof a provided compound or a pharmaceutically acceptable salt thereof and a CHOP (gyclophosphamide, Hydrodaunorubicin®, Qncovin®, and prednisone or prednisolone) or R-CHOP (rituximab, gyclophosphamide, Hydrodaunorubicin®, Qncovin®, and grednisone or Rrednisolone) chemotherapy regimen.

In some embodiments, the present invention provides a method of treating DLBCL comprising administering to a patient in need thereof a provided compound or a pharmaceutically acceptable salt thereof and a rituximab/bendamustine chemotherapy regimen.

In some embodiments, the present invention provides a method of treating DLBCL comprising administering to a patient in need thereof a provided compound or a pharmaceutically acceptable salt thereof and a BTK inhibitor (e.g., ibrutinib).

In some embodiments, the present invention provides a method of treating DLBCL comprising administering to a patient in need thereof a provided compound or a pharmaceutically acceptable salt thereof and an anti-CD20 antibody (e.g., rituximab).

In some embodiments, the present invention provides a method of treating DLBCL comprising administering to a patient in need thereof a provided compound or a pharmaceutically acceptable salt thereof and an anti-CD79B ADC (e.g., polatuzumab).

In some embodiments, the present invention provides a method of treating DLBCL comprising administering to a patient in need thereof a provided compound or a pharmaceutically acceptable salt thereof and a BCL2 inhibitor (e.g., venetoclax).

In some embodiments, the present invention provides a method of treating DLBCL comprising administering to a patient in need thereof a provided compound or a pharmaceutically acceptable salt thereof and lenalidomide or pomalidomide

In some embodiments, the present invention provides a method of treating DLBCL comprising administering to a patient in need thereof a provided compound or a pharmaceutically acceptable salt thereof and a PI3K inhibitor (e.g., umbralisib).

In some embodiments, the present invention provides a method of treating a T-cell disease or deficiency describing herein comprising administering to a patient in need thereof a provided compound or a pharmaceutically acceptable salt thereof and a PI3K inhibitor (e.g., umbralisib).

In some embodiments, the present invention provides a method of treating DLBCL comprising administering to a patient in need thereof a provided compound or a pharmaceutically acceptable salt thereof and a protesome inhibitor (e.g., bortezomib)

In some embodiments, the present invention provides a method of treating a T-cell disease or deficiency describing herein comprising administering to a patient in need thereof a provided compound or a pharmaceutically acceptable salt thereof and a protesome inhibitor (e.g., bortezomib).

In another embodiment, the present invention provides a method of treating multiple myeloma comprising administering to a patient in need thereof a provided compound or a pharmaceutically acceptable salt thereof and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound or a pharmaceutically acceptable salt thereof and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from ConformaTherapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound or a pharmaceutically acceptable salt thereof and one or more additional therapeutic agents selected from donepezil (Aricept), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurfg, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®), Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound or a pharmaceutically acceptable salt thereof and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound or a pharmaceutically acceptable salt thereof and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, the present invention provides a method of treating AML comprising administering to a patient in need thereof a provided compound or a pharmaceutically acceptable salt thereof and one or more additional therapeutic agents selected from: FLT3 inhibitors; targeted agents such as IDH inhibitors, anti-CD33 ADCs (e.g. Mylotarg), BCL2 inhibitors, and Hedgehog inhibitors; and chemotherapy such as AraC, daunarubicin, etoposide, methotrexate, fludarabine, mitozantrone, azacytidine, and corticosteroids.

In some embodiments, the present invention provides a method of treating MDS comprising administering to a patient in need thereof a provided compound or a pharmaceutically acceptable salt thereof and one or more additional therapeutic agents selected from azacytidine, decitabine and revlimid.

In some embodiments, the present invention provides a method of treating inflammatory skin conditions such as hidradenitis suppurativa, comprising administering to a patient in need thereof a provided compound or a pharmaceutically acceptable salt thereof and one or more additional therapeutic agents selected from anti-TNF drugs.

In some embodiments, the present invention provides a method of treating inflammatory skin conditions such as atopic dermatitis, comprising administering to a patient in need thereof a provided compound or a pharmaceutically acceptable salt thereof and one or more additional therapeutic agents selected from IL-4/IL-13-targeted agents such as dupilumab.

In some embodiments, the present invention provides a method of treating inflammatory skin conditions such as psoriasis, comprising administering to a patient in need thereof a provided compound or a pharmaceutically acceptable salt thereof and one or more additional therapeutic agents selected from anti-IL-17 and anti-IL-23 antibodies.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO 2008/118802, US 2010/0197686), navitoclax (and analogs thereof, see U.S. Pat. No. 7,390,799), NH-1 (Shenayng Pharmaceutical University), obatoclax (and analogs thereof, see WO 2004/106328, US 2005/0014802), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SUl01, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/IYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO 2008/039218, US 2008/0108636 and WO 2011/090760, US 2010/0249092, the entirety of each of which is herein incorporated by reference.

Further examples of SYK inhibitory compounds, and conditions treatable by such compounds in combination with compounds of this invention can be found in WO 2003/063794, US 2004/0029902, WO 2005/007623, US 2005/0075306, and WO 2006/078846, US 2006/0211657, the entirety of each of which is herein incorporated by reference.

Further examples of PI3K inhibitory compounds, and conditions treatable by such compounds in combination with compounds of this invention can be found in WO 2004/019973, US 2004/0106569, WO 2004/089925, US 2004/0242631, U.S. Pat. No. 8,138,347, WO 2002/088112, US 2004/0116421, WO 2007/084786, US 2010/0249126, WO 2007/129161, US 2008/0076768, WO 2006/122806, US 2008/0194579, WO 2005/113554, US 2008/0275067, and WO 2007/044729, US 2010/0087440, the entirety of each of which is herein incorporated by reference.

Further examples of JAK inhibitory compounds, and conditions treatable by such compounds in combination with compounds of this invention can be found in WO 2009/114512, US 2009/0233903, WO 2008/109943, US 2010/0197671, WO 2007/053452, US 2007/0191405, WO 2001/0142246, US 2001/0053782, and WO 2007/070514, US 2007/0135461, the entirety of each of which is herein incorporated by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SUl1248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context ofother treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approvided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LT3R, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgGI, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MEDI4736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/0053941, WO 2009/132238, US 2011/0136796, WO 2011/056652, US 2012/0277217, WO 2012/142237, US 2014/0066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (Virallerapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex herapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCTO1862900), head and neck cancer (NCT02274155) and metastatic prostate cancer (NCTO1303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXAMPLES

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1: Salt Screen

A salt screen was conducted with twenty acids in five solvents using amorphous compound 1. In summary of seventy-one wet cake samples fifty six potential crystalline bits were identified by XRPD analysis. Of those fifty six potential hits, fifteen were further characterized by XRPD, DSC, and TGA, and ten of fifteen salt forms displayed good crystallinity and thermal properties.

Salt Forming Experimental:
1) Weigh −50 mg of Compound 1 and 1.0 equivalent acid into a 4.0 mL vial.
2) Add 0.5 ml of solvent.
3) The solution/slurry was agitated with stir bar at RT/300 rpm/3 days
4) Potential crystalline hits were observed by visualization under microscope

TABLE 5
Results of Salt Screen

| Exp. | | | | | | Acetone:H₂O |
|---|---|---|---|---|---|---|
| ID | Acid | MeOH | MeCN | THF | EtOAc | (9:1) |
| 1 | HCl | HCl-1^{b} | HCl-1^{a} | HCl-3^{a} | HCl-3^{a} | HCl-2^{b} |
| 2 | Sulfuric acid (SO4) | Grease | SO4-1^{b} | HSL^{a} | HSL^{a} | Clear |
| 3 | Benzene sulfonic | Grease | BSA-1^{b} | Grease | BSA-1^{a} | Clear |
| | acid (BSA) | | | | | |
| 4 | Tolunenesulfonic | TSA-1^{b} | TSA-1 + TSA-2^{a} | Grease | TSA-2^{a} | Clear |
| | acid monohydrate | | | | | |
| | (TSA) | | | | | |
| 5 | Ethanesulfonic acid | Clear + Grease | ESA-1^{a} | Clear + Grease | ESA-1^{b} | Clear |
| | (ESA) | | | | | |
| 6 | Methanesulfonic | MSA-1^{a} | MSA-2^{a} | MSA-1^{b} | MSA-1^{a} | Clear |
| | acid (MSA) | | | | | |
| 7 | L-Aspartic acid | LAS-1^{a} | LAS-2^{a} | Cloudy^{a} | Slurry^{a} | Clear |
| | (LAS) | | | | | |
| 8 | Maleic acid (Mal) | Grease | Mal-1 + Mal-2^{a} | Mal-1^{b} | Mal-2^{a} | Clear |
| 9 | Phosphoric acid | —^{a} | Amorph.^{a} | Amorph.^{a} | Amorph.^{a} | Clear |
| 10 | L-Glutamic acid | Grease | Counterion^{a} | Counterion^{a} | Counterion^{a} | —^{a} |
| 11 | (+)-L-Tartaric acid | Tar-1^{b} | Tar-2 + Tar-1^{a} | Tar-1^{b} | Slurry^{a} | Clear |
| | (Tar) | | | | | |
| 12 | Fumaric acid | Fum-1^{b} | Fum-1 + minor^{a} | Fum-1 + minor^{a} | Fum-1^{a} | Fum-2^{b} |
| | (Fum) | | | | | |
| 13 | Citric acid (Cit) | Cit-1 + minor^{b} | Cit-1^{b} | Cit-1^{b} | Slurry^{a} | Clear |
| 14 | Glycolic acid (Gly) | Gly-1^{b} | Slurry^{a} | Gly-1^{a} | Gly-1^{b} | Clear |
| 15 | (−)-L-Malic acid | Mali-1^{b} | Mali-3^{a} | Mali-3^{a} | Slurry^{a} | Mali-2^{b} |
| | (Mali) | | | | | |
| 16 | Hippuric acid (Hip) | Hip-1^{b} | Hip-2^{a} | Hip-2^{a} | Hip-3^{a} | Hip-1^{b} |
| 17 | L-Lactic acid (Lac) | Lac-2^{a} | Lac-3^{a} | Lac-4^{a} | Lac-1^{b} | Clear |
| 18 | L-Ascorbic acid | Asc-1^{a} | Asc-1^{a} | TSL^{a} | Slurry^{a} | Clear |
| | (Asc) | | | | | |
| 19 | Benzoic acid (Ben) | Ben-1^{a} | Ben-1^{a} | Ben-1^{a} | Ben-1^{a} | Clear |
| 20 | Succinic acid (Suc) | Suc-2^{b} | Suc-1^{a} | Suc-3^{b} | Suc-1^{a} | Suc-1^{b} |

TSL: thin slurry;
HSL: heavy slurry;
FB: freebase;
^{a}not enough solid;
^{b}crystalline hits analyzed by XRPD.

TABLE 6
Characterization of Salt Screen Hits After Dried

| | | | | | | TGA |
|---|---|---|---|---|---|---|
| | | | | | | (% wt |
| Exp | | | FB:Acid | | | loss up to |
| ID | Acid | Solvent | (Mol. ratio) | XRPD | DSC | 100° C.) |
| 2 | H₂SO₄ | MeCN | Inconclusive | FIG. 1A | 1^{st} endo.: 242.5° C. (onset) | 0.493% |
| 3 | BSA | MeCN | 1:1 | FIG. 2A | 1^{st} endo.: 183.31° C. (onset) | 0.890% |
| 4 | TSA | MeOH | 1:1 | FIG. 3A | 1^{st} endo.: 192.08° C. (onset) | 1.205% |
| | monohydrate | | | | | |
| 5 | ESA | EtOAc | 1:1 | FIG. 4A | 1^{st} endo.: 32.97° C. (onset) | 1.281% |
| | | | | | 2^{nd} endo.: 198.69° C. (onset) | |
| 6 | MSA | THF | 1:1 | FIG. 5A | 1^{st} endo.: 250.41° C. (onset) | 0.344% |
| 8 | Maleic acid | THF | 1:1 | FIG. 6A | 1^{st} endo.: 220.88° C. (onset) | 0.653% |
| 11 | L-Tartaric | MeOH | 2:1 | FIG. 7A | 1^{st} endo.: 252.26° C. (onset) | 1.110% |
| | acid | THF | — | — | — | — |
| 12 | Fumaric acid | MeOH | 2:1 | FIG. 8A | 1^{st} endo.: 268.54° C. (onset) | 0.618% |
| | | Acetone:H₂O | 2:1 | FIG. 8A^{a} | — | — |
| | | (9:1) | | | | |
| 13 | Citric acid | MeOH | 2:1 | FIG. 9A | 1^{st} endo.: 172.49° C. (onset) | 3.202% |
| | | | | | 2^{nd} endo.: 198.24° C. (onset) | |
| | | MeCN | — | — | — | — |
| | | THF | — | — | — | — |
| 14 | Glycolic acid | MeOH | 2:1 | FIG. 10A | 1^{st} endo.: 199.58° C. (onset) | 0.611% |
| | | EtOAc | — | — | — | — |
| 15 | L-Malic acid | MeOH | 2:1 | FIG. 11A | — | — |
| | | Acetone:H₂O | 2:1 | FIG. 11A | 1^{st} endo.: 259.20° C. (onset) | 0.405% |
| | | (9:1) | | | | |
| 16 | Hippuric acid | MeOH | 1:1 | FIG. 12A | 1^{st} endo.: 211.29° C. (onset) | 2.228% |
| | | Acetone:H₂O | — | — | — | — |
| | | (9:1) | | | | |
| 17 | L-Lactic acid | EtOAc | 1:1 | FIG. 13A^{a} | 1^{st} endo.: 172.41° C. (onset) | 0.234% |
| 19 | Benzoic acid | MeOH | 1:1 | FIG. 14A | 1^{st} endo.: 31.91° C. (onset) | 1.760% |
| | | | | | 2^{nd} endo.: 151.75° C. (onset) | |
| 20 | Succinic acid | MeOH | 1:1 | FIG. 15A | 1^{st} endo.: 234.61° C. (onset) | 0.402% |
| | | THF | 1:1 | FIG. 15A | 1^{st} endo.: 140.13° C. (onset) | 0.559% |
| | | | | | 2^{nd} endo.: 186.75° C. (onset) | |
| | | | | | 3^{rd} endo.: 242.36° C. (onset) | |
| | | Acetone:H₂O | 2:1 | FIG. 15A | 1^{st} endo.: 91.61° C. (onset) | 1.271% |
| | | (9:1) | | | 2^{nd} endo.: 244.41° C. (onset) | |

^{a}XRPD changed after drying

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A crystalline salt form of compound 1: 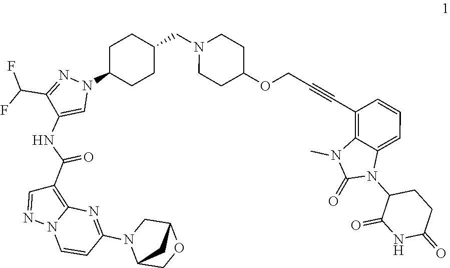 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]
wherein the crystalline salt form is compound 2:

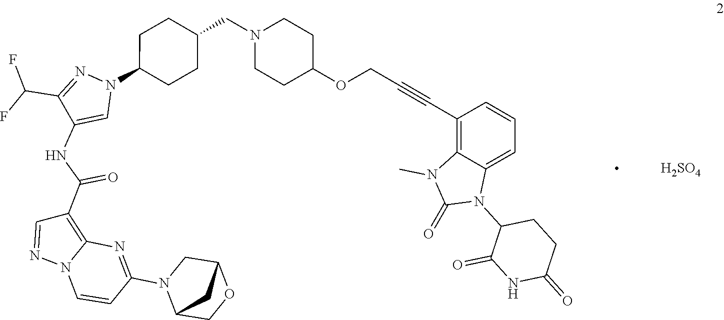 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21 ; "H2SO4"]

2-4. (canceled)

5. The crystalline salt form of claim 1, wherein said salt form is Form A of compound 2.

6. The crystalline salt form of claim 5, wherein Form A of compound 2 is characterized by one or more peaks in its XRPD pattern selected from those at 16.5, 17.5, and 19.7±0.5 degree 2-theta, ±0.4 degree 2-theta.

7. The crystalline salt form of claim 5, having an XRPD substantially as shown in FIG. 1A.

8. The crystalline salt form of claim 5, wherein Form A of compound 2 is characterized by one or more peaks in its XRPD pattern selected from those at 16.5, 17.5, and 19.7±0.3 degree 2-theta.

9. The crystalline salt form of claim 5, wherein Form A of compound 2 is characterized by one or more peaks in its XRPD pattern selected from those at 16.5, 17.5, and 19.7±0.2 degree 2-theta.

10-55. (canceled)

56. A composition comprising a crystalline salt form of claim 1 and a pharmaceutically acceptable carrier or excipient.

57. A method of degrading IRAK4 protein in a patient or biological sample comprising administering to said patient, or contacting said biological sample with a crystalline salt form of claim 1, or a pharmaceutical composition thereof.

58. A method of treating an IRAK4-mediated disorder, disease, or condition in a patient comprising administering to said patient a crystalline salt form of claim 1, or a pharmaceutical composition thereof.

59. The method of claim 58, wherein the IRAK4-mediated disorder, disease or condition is an autoimmune or inflammatory disorder.

60. The method according to claim 59, wherein the autoimmune or inflammatory disorder is selected from the group consisting of ocular allergy, conjunctivitis, keratoconjunctivitis sicca, vernal conjunctivitis; allergic rhinitis, hemolytic anemia, aplastic anemia, pure red cell anemia, idiopathic thrombocytopenia or another inflammatory disease in which autoimmune reactions are implicated or which have an autoimmune component or etiology, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, ulcerative colitis, Crohn's disease or another autoimmune inflammatory bowel disease, irritable bowel syndrome, celiac disease, periodontitis, hyaline membrane disease, kidney disease, glomerular disease, alcoholic liver disease, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, vernal keratoconjunctivitis, interstitial lung fibrosis, psoriatic arthritis, systemic juvenile idiopathic arthritis, nephritis, diverticulitis, interstitial cystitis, glomerulonephritis (with and without nephrotic syndrome, optionally including idiopathic nephrotic syndrome or minal change nephropathy), chronic granulomatous disease, endometriosis, leptospirosis renal disease, glaucoma, retinal disease, aging, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic, non-allergic, mild, moderate, severe, bronchitic, or exercise-induced), acute lung injury, acute respiratory distress syndrome, eosinophilia, hypersensitivities, anaphylaxis, nasal sinusitis, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, Type 2 diabetes, appendicitis, atopic dermatitis, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonia, polymyositis, proctitis, prostatitis, pyelonephritis, rhinitis, salpingitis, sinusitis, stomatitis, synovitis, tendonitis, tonsillitis, vaginitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.
